SEQUENCE LISTING (17 sequences)

SEQ ID NO: 1 - protein, 182 aa, Artificial sequence
  synthetic sequence
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 2 - protein, 182 aa, Artificial sequence
  synthetic sequence
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 3 - protein, 297 aa, Artificial sequence
  synthetic sequence
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 4 - protein, 5 aa, Artificial sequence
  synthetic sequence
  SITE LOCATION: (2)..(3): Xaa is any amino acid
VXXSL

SEQ ID NO: 5 - protein, 5 aa, Artificial sequence
  synthetic sequence
VKESL

SEQ ID NO: 6 - protein, 5 aa, Artificial sequence
  synthetic sequence
VLGSL

SEQ ID NO: 7 - protein, 16 aa, Artificial sequence
  synthetic sequence
NANSFCYENEVALTSK

SEQ ID NO: 8 - protein, 6 aa, Artificial sequence
  synthetic sequence
  SITE LOCATION: (2)..(2): Xaa is any amino acid
FXYENE

SEQ ID NO: 9 - protein, 7 aa, Artificial sequence
  synthetic sequence
FCYENEV

SEQ ID NO: 10 - protein, 44 aa, Artificial sequence
  synthetic sequence
  SITE LOCATION: (12)..(16): The amino acids at positions 12 to 16 may either be present or absent
  SITE LOCATION: (17)..(21): The amino acids at positions 17 to 21 may either be present or absent
  SITE LOCATION: (23)..(44): The amino acids at positions 23 to 44 may either be present or absent
AEAAAKEAAAKEAAAKEAAAKAAEAAAKEAAAKEAAAKEAAAKA

SEQ ID NO: 11 - protein, 20 aa, Artificial sequence
  synthetic sequence
  SITE LOCATION: (1)..(1): Xaa is any amino acid. In some cases, Xaa is Ala, Lys or Glu
  SITE LOCATION: (3)..(4): The amino acids at positions 3 to 4 are either present or absent
  SITE LOCATION: (3)..(3): Xaa is any amino acid. In some cases, Xaa is Ala, Lys or Glu
  SITE LOCATION: (5)..(6): The amino acids at positions 5 to 6 are either present or absent
  SITE LOCATION: (5)..(5): Xaa is any amino acid. In some cases, Xaa is Ala, Lys or Glu
  SITE LOCATION: (7)..(8): The amino acids at positions 7 to 8 are either present or absent
  SITE LOCATION: (7)..(7): Xaa is any amino acid. In some cases, Xaa is Ala, Lys or Glu
  SITE LOCATION: (9)..(10): The amino acids at positions 9 to 10 are either present or absent
  SITE LOCATION: (9)..(9): Xaa is any amino acid. In some cases, Xaa is Ala, Lys or Glu
  SITE LOCATION: (11)..(12): The amino acids at positions 11 to 12 are either present or absent
  SITE LOCATION: (11)..(11): Xaa is any amino acid. In some cases, Xaa is Ala, Lys or Glu
  SITE LOCATION: (13)..(14): The amino acids at positions 13 to 14 are either present or absent
  SITE LOCATION: (13)..(13): Xaa is any amino acid. In some cases, Xaa is Ala, Lys or Glu
  SITE LOCATION: (15)..(16): The amino acids at positions 15 to 16 are either present or absent
  SITE LOCATION: (15)..(15): Xaa is any amino acid. In some cases, Xaa is Ala, Lys or Glu
  SITE LOCATION: (17)..(18): The amino acids at positions 17 to 18 are either present or absent
  SITE LOCATION: (17)..(17): Xaa is any amino acid. In some cases, Xaa is Ala, Lys or Glu
  SITE LOCATION: (19)..(20): The amino acids at positions 19 to 20 are either present or absent
  SITE LOCATION: (19)..(19): Xaa is any amino acid. In some cases, Xaa is Ala, Lys or Glu
XPXPXPXPXPXPXPXPXPXP

SEQ ID NO: 12 - protein, 30 aa, Artificial sequence
  synthetic sequence
  SITE LOCATION: (6)..(10): The amino acids at positions 6 to 10 may either be present or absent
  SITE LOCATION: (11)..(15): The amino acids at positions 11 to 15 may either be present or absent
  SITE LOCATION: (16)..(20): The amino acids at positions 16 to 20 may either be present or absent
  SITE LOCATION: (21)..(25): The amino acids at positions 21 to 25 may either be present or absent
  SITE LOCATION: (26)..(30): The amino acids at positions 26 to 30 may either be present or absent
EAAAKEAAAKEAAAKEAAAKEAAAKEAAAK

SEQ ID NO: 13 - protein, 5 aa, Artificial sequence
  synthetic sequence
EAAAK

SEQ ID NO: 14 - protein, 5 aa, Artificial Sequence
  synthetic sequence
GGGGS

SEQ ID NO: 15 - protein, 15 aa, Artificial sequence
  synthetic sequence
EAAAKEAAAKEAAAK

SEQ ID NO: 16 - protein, 5 aa, Artificial sequence
  synthetic sequence
  SITE LOCATION: (1)..(5): The sequence comprises a membrane anchor (M)
EAAAK

SEQ ID NO: 17 - protein, 5 aa, Artificial sequence
  synthetic sequence
  SITE LOCATION: (1)..(5): The sequence comprises a membrane anchor (M) and an endoplasmic reticulum export (ERE) motif
EAAAK

(19) United States
(12) Patent Application Publication
(10) Pub. No.: US 2023/0173090 A1
(43) Pub. Date: Jun. 8, 2023

(54) COMPOSITIONS AND METHODS FOR MODULATING DOPAMINE RECEPTOR ACTIVITY

(71) Applicant: The Regents of the University of California, Oakland, CA (US)

(72) Inventors: Ehud Y. Isacoff, Berkeley, CA (US); Prashant Chandra Donthamsetti, Oakland, CA (US)

(21) Appl. No.: 17/928,850

(22) PCT Filed: Jun. 29, 2021
(86) PCT No.: PCT/US2021/039557

§ 371 Date: Nov. 30, 2022

Related U.S. Application Data
(60) Provisional application No. 63/046,329, filed on Jun. 30, 2020.

(51) Int. Cl. A61K 47/68; A61K 47/64; A61K 47/65; A61K 41/00; A61K 47/54

(52) U.S. Cl. CPC A61K 47/6849; A61K 41/00; A61K 47/65; A61K 47/545; A61K 47/6425

(57) ABSTRACT

The present disclosure provides conjugates and systems for modulating the activity of a ligand-binding polypeptide such as a D1 dopamine receptor. The present disclosure provides methods of modulating the activity of a D1 dopamine receptor. The present disclosure provides methods of treating Parkinson’s disease in an individual.

### CROSS-REFERENCE

This application claims the benefit of U.S. Provisional Pat. Application No. 63/046,329, filed Jun. 30, 2020, which application is incorporated herein by reference in its entirety.

### STATEMENT REGARDING FEDERALLY SPONSORED RESEARCH

This invention was made with government support under Grant Numbers EY016554 and EY018241 awarded by the National Institutes of Health. The government has certain rights in the invention.

### INTRODUCTION

Dopamine (DA) circuits play important roles in health (movement, reward, aversion, motivation) and disease (e.g., Parkinson’s disease, schizophrenia, and addiction). DA neurons have diverse downstream targets, making it difficult to identify the casual basis of specific behavioral outcomes. For example, the striatum is a hub region that receives dense dopaminergic innervation from the midbrain. Multiple distinct, spatially intermixed and inter-connected striatal cell types express various combinations of four of the five DA receptor (DAR) subtypes, the G_{s/olf}-coupled D1-like receptors D1R and D5R and the G_{i/o/z}-coupled D2-like receptors D2R and D3R.

There is a need in the art for methods that can control specific DARs.

### SUMMARY

The present disclosure provides conjugates and systems for modulating the activity of a ligand-binding polypeptide such as a D1 dopamine receptor. The present disclosure provides methods of modulating the activity of a D1 dopamine receptor. The present disclosure provides methods of treating Parkinson’s disease in an individual.

### BRIEF DESCRIPTION OF THE DRAWINGS

FIGS. 1A-1C depict design of a D1R MP.

FIGS. 2A-2I depict photoactivation of D1R by MP-D1_{ago}.

FIGS. 3A-3G depict the effect of activation of D1Rs in MSNs of the dStr with MP-D1_{ago} on movement.

FIGS. 4A-4F depict the effect of dStr-dMSN D1R activation with MP-D1_{ago} on movement initiation and movement duration.

FIGS. 5A-5I depict a comparison of the motor effect of optogenetic activation of SNc terminals in the dStr with ChR2 to dStr-dMSN D1R activation with MP-D1_{ago}.

FIGS. 6A-6B depict chemical synthesis of P-D1_{ago}.

FIGS. 7A-7E depict design of P-D1_{ago}.

FIGS. 8A-8F depict photophysical properties of P-D1_{ago}.

FIGS. 9A-9H depict functional properties of untethered P-D1_{ago}.

FIGS. 10A-10C depict functional characterization of the effect of P-D1_{ago} tethered to SNAP-D1R.

FIGS. 11A-11F depict functional characterization of the effect of P-D1_{ago} tethered to M or its variants on D1R.

FIGS. 12A-12K depict showing that increasing the surface level of membrane-anchored P-D1_{ago} enhances D1R photoactivation without increasing basal receptor activation.

FIGS. 13A-13G depict functional properties of MP-D1_{ago} compared to opto-D1R.

FIGS. 14A-14M depict data showing that MP-D1_{ago} is a D1R/D5R selective-photoagonist.

FIGS. 15A-15D depict expression of the membrane anchor component of MP-D1_{ago}, M_{EAAAK}:_{ERE} (SEQ ID NO: 17).

FIGS. 16A-16G depict design and optimization of a bilateral infusion and dual color light delivery system for mouse brain.

FIGS. 17A-17E depict data for the motor effect of MP-D1_{ago}-induced activation of dStr-dMSN D1Rs.

FIGS. 18A-18F depict data for the movement effects of dStr-dMSN D1R activation with MP-D1_{ago}.

FIGS. 19A-19H depict use of MP-D1_{ago} to chronically activate dStr-dMSN D1Rs.

FIGS. 20A-20H depict data for the motor effect of optogenetic activation of SNc terminals in the dStr with ChR2.

FIGS. 21A-21K depict comparison of the motor effect of a dStr-infusion of the full D1R agonist SKF82958 and dStr-dMSN D1R activation with MP-D1_{ago}.

FIGS. 22A-22F depict the effect of activation of D1Rs in MSNs of the vStr with MP-D1_{ago} on movement.

FIG. 23 depicts a D1 photo-antagonist (“P-D1_{block}”), a D2-specific photo-agonist (“P-D2_{ago}”), and a D2-specific photo-antagonist (“P-D2_{block}”).

FIG. 24 depicts the effect of a P-D 1_{block} on HEK293 cells co-expressing the membrane anchor (M), SNAP-TM, along with D1R and the GIRK channel.

### DEFINITIONS

The term “alkyl” refers to a monoradical branched or unbranched saturated hydrocarbon chain, e.g., having from 1 to 40 carbon atoms, from 1 to 10 carbon atoms, or from 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined above wherein one or more carbon atoms in the alkyl chain have been optionally replaced with a heteroatom such as —O—, —S(O)ₙ— (where n is 0 to 2), -NR- (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-aryl, -SO-heteroaryl, -SOz-alkyl, -SOz-aryl, -SO₂-heteroaryl, and -NR^{a}Rb, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

The term “alkylaminoalkyl”, “alkylaminoalkenyl” and “alkylaminoalkynyl” refers to the groups R^{a}NHR^{b}- where R^{a} is alkyl group as defined above and R^{b} is alkylene, alkenylene or alkynylene group as defined above.

The term “alkaryl” or “aralkyl” refers to the groups -alkylene-aryl and -substituted alkylene-aryl where alkylene, substituted alkylene and aryl are defined herein.

The term “alkoxy” refers to the groups alkyl-O, alkenyl-O-, cycloalkyl-O-, cycloalkenyl-O-, and alkynyl-O-, where alkyl, alkenyl, cycloalkyl, cycloalkenyl, and alkynyl are as defined herein.

The term “substituted alkoxy” refers to the groups substituted alkyl-O-, substituted alkenylx-O-, substituted cycloalkyl-O-, substituted cycloalkenyl-O-, and substituted alkynyl-O-where substituted alkyl, substituted alkenyl, substituted cycloalkyl, substituted cycloalkenyl and substituted alkynyl are as defined herein.

The term “haloalkoxy” refers to the groups alkyl-O- wherein one or more hydrogen atoms on the alkyl group have been substituted with a halo group and include, by way of examples, groups such as trifluoromethoxy, and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

The term “alkenyl” refers to a monoradical of a branched or unbranched unsaturated hydrocarbon group having from 2 to 40 carbon atoms, from 2 to 10 carbon atoms, or from 2 to 6 carbon atoms and having at least 1 site (e.g., from 1-6 sites) of vinyl unsaturation.

The term “substituted alkenyl” refers to an alkenyl group as defined above having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-substituted alkyl, -SO-aryl, -SO-heteroaryl, -SOz-alkyl, -SO₂-substituted alkyl, -SO₂-aryl and -SO₂-heteroaryl.

The term “alkynyl” refers to a monoradical of an unsaturated hydrocarbon having from 2 to 40 carbon atoms, from 2 to 20 carbon atoms, or from 2 to 6 carbon atoms and having at least 1 site (e.g., from 1-6 sites) of acetylene (triple bond) unsaturation.

The term “substituted alkynyl” refers to an alkynyl group as defined above having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-substituted alkyl, -SO-aryl, -SO-heteroaryl, -SO₂-alkyl, -SO₂-substituted alkyl, -SO₂-aryl, and -SO₂-heteroaryl.

The term “acyl” refers to the groups HC(O)—, alkyl-C(O)-, substituted alkyl-C(O)-, cycloalkyl-C(O)-, substituted cycloalkyl-C(O)-, cycloalkenyl-C(O)-, substituted cycloalkenyl-C(O)-, aryl-C(O)-, heteroaryl-C(O)- and heterocyclic-C(O)- where alkyl, substituted alkyl, cycloalkyl, substituted cycloalkyl, cycloalkenyl, substituted cycloalkenyl, aryl, heteroaryl, and heterocyclic are as defined herein.

The term “acylamino” or “aminocarbonyl” refers to the group -C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl, and heterocyclic are as defined herein.

The term “aminoacyl” refers to the group -NRC(O)R where each R is independently hydrogen, alkyl, substituted alkyl, aryl, heteroaryl, or heterocyclic wherein alkyl, substituted alkyl, aryl, heteroaryl, and heterocyclic are as defined herein.

The term “aminoacyloxy” or “alkoxycarbonylamino” refers to the group -NRC(O)OR where each R is independently hydrogen, alkyl, substituted alkyl, aryl, heteroaryl, or heterocyclic wherein alkyl, substituted alkyl, aryl, heteroaryl, and heterocyclic are as defined herein.

The term “acyloxy” refers to the groups alkyl—C(O)O—, substituted alkyl-C(O)O-, cycloalkyl-C(O)O-, substituted cycloalkyl-C(O)O-, aryl-C(O)O-, heteroaryl-C(O)O-, and heterocyclic-C(O)O- wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Exemplary aryls include phenyl, naphthyl and the like. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, -SO-alkyl, -SO-substituted alkyl, -SO-aryl, -SO-heteroaryl, -SOz-alkyl, -SO₂-substituted alkyl, -SO₂-aryl, -SO₂-heteroaryl and trihalomethyl.

The term “aryloxy” refers to the group aryl-O- wherein the aryl group is as defined above including optionally substituted aryl groups as also defined herein.

The term “amino” refers to the group —NH₂.

The term “substituted amino” refers to the group -NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclic provided that both R’s are not hydrogen.

The term “carboxyalkyl” or “alkoxycarbonyl” refers to the groups “-C(O)O-alkyl”, “-C(O)O-substituted alkyl”, “-C(O)O-cycloalkyl”, “-C(O)O-substituted cycloalkyl”, “-C(O)O-alkenyl”, “-C(O)O-substituted alkenyl”, “-C(O)O-alkynyl” and “-C(O)O-substituted alkynyl” where alkyl, substituted alkyl, cycloalkyl, substituted cycloalkyl, alkenyl, substituted alkenyl, alkynyl and substituted alkynyl alkynyl are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-substituted alkyl, -SO-aryl, -SO-heteroaryl, -SOz-alkyl, -SO₂-substituted alkyl, -SO₂-aryl and -SO₂-heteroaryl.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl, and the like.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-substituted alkyl, -SO-aryl, -SO-heteroaryl, -SO₂-alkyl, -SO₂-substituted alkyl, -SO₂-aryl and -SO₂-heteroaryl.

The term “halo” or “halogen” refers to fluoro, chloro, bromo and iodo.

The term “heteroaryl” refers to an aromatic group of from 1 to 15 carbon atoms and 1 to 4 heteroatoms selected from oxygen, nitrogen and sulfur within at least one ring (if there is more than one ring). Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, -SO-alkyl, -SO-substituted alkyl, -SO-aryl, -SO-heteroaryl, -SOz-alkyl, -SO₂-substituted alkyl, -SO₂-aryl and -SO₂-heteroaryl, and trihalomethyl.

The term “heteroaralkyl” refers to the groups -alkylene-heteroaryl where alkylene and heteroaryl are defined herein. Such heteroaralkyl groups are exemplified by pyridylmethyl, pyridylethyl, indolylmethyl, and the like.

The term “heteroaryloxy” refers to the group heteroaryl-O-.

The term “heterocycle” or “heterocyclic” refers to a monoradical saturated or unsaturated group having a single ring or multiple condensed rings, from 1 to 40 carbon atoms and from 1 to 10 hetero atoms, e.g., from 1 to 4 heteroatoms, selected from nitrogen, sulfur, phosphorus, and/or oxygen within the ring. Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, -SO-alkyl, -SO-substituted alkyl, -SO-aryl, -SO-heteroaryl, -SO₂-alkyl, -SO₂-substituted alkyl, -SO₂-aryl and -SO₂-heteroaryl.

Examples of nitrogen heteroaryls and heterocycles include, but are not limited to, pyrrole, thiophene, furan, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, pyrrolidine, piperidine, piperazine, indoline, morpholine, tetrahydrofuranyl, tetrahydrothiophene, and the like as well as N-alkoxy-nitrogen containing heterocycles.

The term “heterocyclooxy” refers to the group heterocyclic-O-.

The term “heterocyclothio” refers to the group heterocyclic-S-.

The term “heterocyclene” refers to the diradical group formed from a heterocycle, as defined herein, and is exemplified by the groups 2,6-morpholino, 2,5-morpholino and the like.

The term “heteroarylamino” refers to a 5 membered aromatic ring wherein one or two ring atoms are N, the remaining ring atoms being C. The heteroarylamino ring may be fused to a cycloalkyl, aryl or heteroaryl ring, and it may be optionally substituted with one or more substituents, e.g., one or two substituents, selected from alkyl, substituted alkyl, cycloalkyl, aryl, aralkyl, heteroaryl, heteroaralkyl, halo, cyano, acyl, amino, substituted amino, acylamino, -OR (where R is hydrogen, alkyl, alkenyl, cycloalkyl, acyl, aryl, heteroaryl, aralkyl, or heteroaralkyl), or -S(O)ₙR where n is an integer from 0 to 2 and R is hydrogen (provided that n is 0), alkyl, alkenyl, cycloalkyl, amino, heterocyclo, aryl, heteroaryl, aralkyl, or heteroaralkyl.

The term “heterocycloamino” refers to a saturated monovalent cyclic group of 4 to 8 ring atoms, wherein at least one ring atom is N and optionally contains one or two additional ring heteroatoms selected from the group consisting of N, O, or S(O)n (where n is an integer from 0 to 2), the remaining ring atoms being C, where one or two C atoms may optionally be replaced by a carbonyl group. The heterocycloamino ring may be fused to a cycloalkyl, aryl or heteroaryl ring, and it may be optionally substituted with one or more substituents, e.g., one or two substituents, selected from alkyl, substituted alkyl, cycloalkyl, aryl, aralkyl, heteroaryl, heteroaralkyl, halo, cyano, acyl, amino, substituted amino, acylamino, -OR (where R is hydrogen, alkyl, alkenyl, cycloalkyl, acyl, aryl, heteroaryl, aralkyl, or heteroaralkyl), or -S(O)ₙR [where n is an integer from 0 to 2 and R is hydrogen (provided that n is 0), alkyl, alkenyl, cycloalkyl, amino, heterocyclo, aryl, heteroaryl, aralkyl, or heteroaralkyl].

The term “oxyacylamino” or “aminocarbonyloxy” refers to the group -OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “thiol” refers to the group —SH.

The term “thioalkoxy” or “alkylthio” refers to the group -S-alkyl.

The term “substituted thioalkoxy” refers to the group -S-substituted alkyl.

The term “thioaryloxy” refers to the group aryl-S- wherein the aryl group is as defined above including optionally substituted aryl groups also defined above.

The term “thioheteroaryloxy” refers to the group heteroaryl-S- wherein the heteroaryl group is as defined above including optionally substituted aryl groups as also defined above.

As to any of the above groups which contain one or more substituents, it is understood, of course, that such groups do not contain any substitution or substitution patterns which are sterically impractical and/or synthetically non-feasible. In addition, the compounds of the embodiments include all stereochemical isomers arising from the substitution of these compounds.

The term “pharmaceutically-acceptable salt” refers to salts which retain biological effectiveness and are not biologically or otherwise undesirable. In many cases, the compounds of the embodiments are capable of forming acid and/or base salts by virtue of the presence of amino and/or carboxyl groups or groups similar thereto.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component.

A polypeptide has a certain percent “sequence identity” to another polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wisconsin, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and
Joining Penalty: 30.0.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

The terms “polyethylene oxide”, “PEO”, “polyethylene glycol” and “PEG” are used interchangeably and refer to a polymeric group including a chain described by the formula --(CH₂-CH₂—O—)ₙ₋ or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15.

The terms “antibodies” and “immunoglobulin” include antibodies or immunoglobulins of any isotype, fragments of antibodies that retain specific binding to antigen (e.g., to a target ligand-binding polypeptide), including, but not limited to, Fab, Fv, scFv, and Fd fragments, chimeric antibodies, humanized antibodies, single-chain antibodies (scAb), single domain antibodies (sdAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids. In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

Cartilaginous fishes also have heavy-chain antibodies (IgNAR; “immunoglobulin new antigen receptor”), from which single-domain antibodies called V_{NAR} fragments can be obtained. Thus, in some cases, an affinity agent is an IgNAR.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al. (1995) Protein Eng. 8(10): 1057-1062); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen. Antibody fragments include, e.g., scFv, sdAb, dAb, Fab, Fab′, Fab′₂, F(ab′)₂, Fd, Fv, Feb, and SMIP. Examples of sdAb are a camelid VHH and a cartilaginous fish VNAR.

“Fv” is the minimum antibody fragment that contains a complete antigen-recognition and -binding site. This region consists of a dimer of one heavy- and one light-chain variable domain in tight, non-covalent association. It is in this configuration that the three complementarity determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; (c) relieving the disease, i.e., causing regression of the disease; and (d) replacing a lost function that results from the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a ligand” includes a plurality of such ligands and reference to “the D1 dopamine receptor agonist” includes reference to one or more D1 dopamine receptor agonists and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides conjugates and systems for modulating the activity of a ligand-binding polypeptide such as a D1 dopamine receptor. The present disclosure provides methods of modulating the activity of a D1 dopamine receptor. The present disclosure provides methods of treating Parkinson’s disease in an individual.

### Systems

The present disclosure provides systems for modulating a D1 dopamine receptor in an individual. A system of the present disclosure includes: a) a conjugate comprising: i) an affinity agent that forms a covalent bond with a self-labeling protein tag; ii) a linker; iii) a photoisomerizable group; and iv) a ligand that binds to a target ligand-binding polypeptide. A system of the present disclosure also includes either: b1) a fusion polypeptide, or a recombinant expression vector comprising a nucleotide sequence encoding the fusion polypeptide, wherein the fusion polypeptide comprises: i) a self-labeling protein tag; ii) a peptide linker; and iii) a membrane-anchoring polypeptide; or b2) a fusion polypeptide, or a recombinant expression vector comprising a nucleotide sequence encoding the fusion polypeptide, wherein the fusion polypeptide comprises: i) a self-labeling protein tag; and ii) an antibody specific for a D1 dopamine receptor.

### Conjugates

A conjugate for inclusion in a system of the present disclosure comprises i) an affinity agent that forms a covalent bond with a self-labeling protein tag; ii) a linker; iii) a photoisomerizable group; and iv) a ligand that binds to a target ligand-binding polypeptide.

In some cases, a conjugate suitable for inclusion in a system of the present disclosure is a compound having the formula: (A)-X₁-(B)-X₂-(C), where:
A is an affinity agent;
B is a photoisomerizable group;
C is a ligand;
X₁, when present, is a linker; and
X₂, when present, is a linker.

### Affinity Agents

Affinity agents suitable for inclusion in a conjugate include agents that bind to self-labeling polypeptides. Suitable affinity agents include nucleoside base derivatives. In some cases, the nucleoside base of the nucleoside base derivative is selected from guanine, cytosine, uracil, thymine, xanthine, and hypoxanthine. For example, the nucleoside base of the nucleoside base derivative can be guanine, xanthine or hypoxanthine. In some cases, the nucleoside base of the nucleoside base derivative is guanine. In other instances, the nucleoside base of the nucleoside base derivative can be cytosine, thymine or uracil. In some cases, the nucleoside base of the nucleoside base derivative is cytosine. The nucleoside base can be derivatized to provide the nucleoside base derivative of the affinity agent of a conjugate of the present disclosure. In some cases, the nucleoside base derivative of the affinity agent is a benzylnucleoside base, such as benzylguanine or benzylcytosine. In some cases, the affinity agent is benzylguanine. In embodiments where the affinity agent is benzylguanine, the benzylguanine affinity agent may provide for covalent binding to a SNAP tag. In some cases, the affinity agent is benzylcytosine. In embodiments where the affinity agent is benzylcytosine, the benzylcytosine affinity agent may provide for covalent binding to a CLIP tag. In some cases, the affinity agent is a chloropyrimidine; a chloropyrimidine can bind to a SNAP tag.

Suitable affinity agents also include alkyl derivatives, such as haloalkyl derivatives where one or more hydrogen atoms in an alkyl or alkyl derivative is replaced by a halogen, e.g., fluoro, chloro, or bromo. In some cases, the haloalkyl derivative is a fluoroalkane. In some cases, the haloalkyl derivative is a chloroalkane. In some cases, the haloalkyl derivative is a bromoalkane. In some cases, the affinity agent is chloroalkane, such as C1(CH₂)₆(OCH₂CH₂)₂. In embodiments where the affinity agent is chloroalkane, the chloroalkane affinity agent may provide for covalent binding to a HALO tag.

### Photoisomerizable Groups

Photoisomerizable groups are known in the art, and any known photoisomerizable group can be included in the photoisomerizable regulator present in a conjugate of the present disclosure. Suitable photoisomerizable groups include, but are not limited to, azobenzene, cyclic azobenzenes and azoheteroarenes and derivatives thereof; spiropyran and derivatives thereof; triphenyl methane and derivatives thereof; 4,5-epoxy-2-cyclopentene and derivatives thereof; fulgide and derivatives thereof; thioindigo and derivatives thereof; diarylethene and derivatives thereof; diallylethene and derivatives thereof; overcrowded alkenes and derivatives thereof; and anthracene and derivatives thereof. In some cases, a suitable photoisomerizable group is a photoisomerizable group as shown in the examples herein.

Suitable spiropyran derivatives include, but are not limited to, 1,3,3-trimethylindolinobenzopyrylospiran; 1,3,3-trimethylindolino-6′-nitrobenzopyrylospiran; 1,3,3-trimethylindolino-6′-bromobenzopyrylospiran; 1-n-decyl-3,3-dimethylindolino-6′-nitrobenzopyrylospiran; 1-n-octadecy-1-3,3-dimethylindolino-6′-nitrobenzopyrylospiran; 3′,3′-dimethyl-6-nitro-1′-[2-(phenylcarbamoyl)ethyl]spiro; [2H-1-benzopyran-2,2′-indoline]; 1,3,3-trimetnylindolino-8′-methoxybenzopyrylospiran; and 1,3,3-trimethylindolino-β-naphthopyrylospiran. Also suitable for use is a merocyanine form corresponding to spiropyran or a spiropyran derivative.

Suitable triphenylmethane derivatives include, but are not limited to, malachite green derivatives, specifically, there can be mentioned, for example, bis[dimethylamino)phenyl] phenylmethanol, bis[4-(diethylamino)phenyl]phenylmethanol, bis[4-(dibuthylamino)phenyl]phenylmethanol and bis[4-(diethylamino)phenyl]phenylmethane.

Suitable 4,5-epoxy-2-cyclopentene derivatives include, for example, 2,3-diphenyl-1-indenone oxide and 2′,3′-dimethyl-2,3-diphenyl-1-indenone oxide.

Suitable azobenzene compounds include, e.g., compounds having azobenzene residues crosslinked to a side chain, e.g., compounds in which 4-carboxyazobenzene is ester bonded to the hydroxyl group of polyvinyl alcohol or 4-carboxyazobenzene is amide bonded to the amino group of polyallylamine. Also suitable are azobenzene compounds having azobenzene residues in the main chain, for example, those formed by ester bonding bis(4-hydroxyphenyl)dimethylmethane (also referred to as bisphenol A) and 4,4′-dicarboxyazobenzene or by ester bonding ethylene glycol and 4,4′-dicarboxyazobenzene.

Suitable cyclic azobenzene and azoheteroarene compounds which can be adapted for use in the subject conjugates and photoisomerizable regulators include, but are not limited to, 11,12-dihydrodibenzo[c,g] [1,2]diazocine-5-oxide,

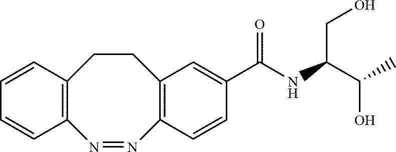 [SMILES: C[C@H](O)[C@H](CO)NC(=O)c1ccc2c(c1)CCc1ccccc1/N=N\2]

, heterodiazocines, such as those photoswitches described by Hammerich et al. J. Am. Chem. Soc., 2016, 138 (40), pp 13111-13114), and azoheteroarene photoswitches such as 3-pyrazoles (3 pzH or 3 pzMe), 5-pyrazoles (5 pzH or 5 pzMe), 3-pyrrroles (3 pyH or 3 pyMe), triazole and tetrazoles (tet or 3 tri) as describes by Calbo et al. J. Am. Chem. Soc., 2017, 139 (3), pp 1261-1274, the disclosure of which is herein incorporated by reference.

Suitable fulgide derivatives include, but are not limited to, isopropylidene fulgide and adamantylidene fulgide.

Suitable diallylethene derivatives include, for example, 1,2-dicyano-1,2-bis(2,3,5-trimethyl-4-thienyl)ethane; 2,3-bis(2,3,5-trimethyl-4-thiethyl) maleic anhydride; 1,2-dicyano-1,2-bis(2,3,5-trimethyl-4-selenyl)ethane; 2,3-bis(2,3,5-trimethyl-4-selenyl) maleic anhydride; and 1,2-dicyano-1,2-bis(2-methyl-3-N-methylindole)ethane.

Suitable diarylethene derivatives include but are not limited to, substituted perfluorocylopentene-bis-3-thienyls and bis-3-thienylmaleimides.

Suitable overcrowded alkenes include, but are not limited to, cis-2-nitro-7-(dimethylamino)-9-(2’,3′-dihydro-1’H-naphtho[2,1-b]thiopyran-1′-ylidene)-9H-thioxanthene and trans-dimethyl-[1-(2-nitro-thioxanthen-9-ylidene)-2,3 -dihydro-1 H-benzo [f] thiochromen- 8 -yl]amine. Overcrowded alkenes are described in the literature. See, e.g., terWiel et al. (2005) Org. Biomol. Chem. 3:28-30; and Geertsema et al. (1999) Agnew CHem. Int. Ed. Engl. 38:2738.

Other suitable photoisomerizable groups include, e.g., reactive groups commonly used in affinity labeling, including diazoketones, aryl azides, diazerenes, and benzophenones.

In some instances, the photoisomerizable group of the conjugate (e.g., as defined herein) is an azobenzene (e.g., an azobenzene photoswitch) of the following formula:

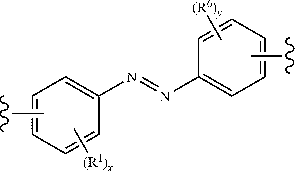

wherein:
R¹ and R⁶ are one or more optional substituents selected from hydrogen, C₁₋₁₀ alkyl, substituted C₁₋₁₀ alkyl, -NR¹⁰R¹¹ -NR¹²C(O)R¹³, -NR¹²C(O)OR¹³, -NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C4-10 cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
x is an integer from 1 to 5;
y is an integer from 1 to 5; and
wherein R¹⁰-R¹³ are as defined below,
or a pharmaceutically acceptable salt thereof.

In some cases, a photoisomerizable group present in a conjugate of the present disclosure is a compound of Formula 1:

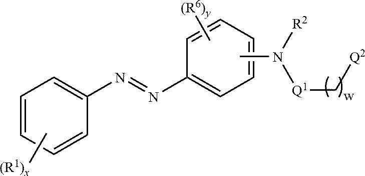

wherein:
Q¹ is —CH₂— or —C(═O)—;
Q² is a ligand (or a label or reactive group or second affinity agent), as described according to the present disclosure;
each R¹ is independently selected from hydrogen, C₁₋₁₀ alkyl, substituted C₁₋₁₀ alkyl, -NR¹⁰R¹¹ -NR¹²C(O)R¹³, -NR¹²C(O)OR¹³, -NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C4-10 cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; each R⁶ is independently selected from hydrogen, C₁₋₁₀ alkyl, substituted C₁₋₁₀ alkyl, -NR¹⁰R¹¹, -NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
R′° and R¹¹ are each independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃^{—};
or a pharmaceutically acceptable salt thereof.

In certain embodiments of Formula 1, Q¹ is —CH₂—. In certain embodiments of Formula 1, Q¹ is —C(═O)—.

In some instances of Formula 1, one of R¹ is linked via a linker to an affinity agent (e.g., as described herein). In some cases, the linker includes a branched linker (e.g., as described herein).

In some cases, a photoisomerizable group present in a conjugate of the present disclosure is a compound of Formula 2:

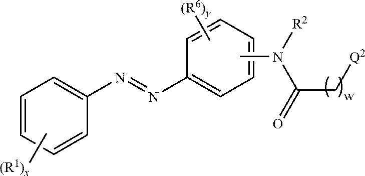

wherein
Q² is a ligand (or a label or reactive group or second affinity agent), as described according to the present disclosure;
each R¹ is independently selected from hydrogen, C₁₋₁₀ alkyl, substituted C₁₋₁₀ alkyl, -NR¹⁰R¹¹ -NR¹²C(O)R¹³, -NR¹²C(O)OR¹³, -NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R₁₀;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, -NR¹⁰R1 1, -NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
R¹⁰ and R¹¹ are each independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C6-20 aryl, substituted C6-20 aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, -CH₂—N(CH₂CH₃)³⁺, and —CH₂—SO₃^{—};
or a pharmaceutically acceptable salt thereof.

In some instances of Formula 2, one of the R¹ groups is linked via a linker to an affinity agent (e.g., as described herein). In some cases, the linker includes a branched linker (e.g., as described herein).

In some cases, a photoisomerizable group present in a conjugate of the present disclosure is a compound of Formula 3:

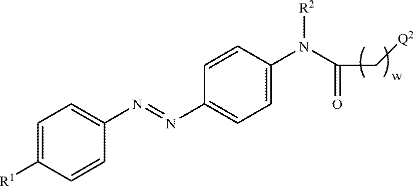

wherein:
Q² is a ligand (or a label or reactive group or second affinity agent), as described according to the present disclosure;
w is an integer from 1 to 10;
R¹ is selected from hydrogen, C₁₋₁₀ alkyl, -NR¹⁰R¹¹ -NR¹²C(O)R¹³, -NR¹²C(O)OR¹³ and -NR¹²C(O)NR¹²R¹³;
R² is hydrogen or C₁₋₁₀ alkyl;
R¹⁰ and R¹¹ are independently selected from hydrogen and C₁₋₁₀ alkyl;
R¹² is hydrogen or C₁₋₁₀ alkyl; and
R¹³ is selected from hydrogen, C₁₋₁₀ alkyl, C₁₋₈ alkenyl, C₆₋₁₀ aryl, and substituted C₁₋₁₀ alkyl,
or a pharmaceutically acceptable salt thereof.

In certain embodiments of Formula 3, R¹ is C₁₋₁₀ alkyl, such as C₁₋₈ alkyl, e.g., C₁₋₆ alkyl, C₁₋₅ alkyl or C₁₋₄ alkyl. In some embodiments of Formula 3, R¹ is C₁₋₄ alkyl.

In certain embodiments of Formula 3, R¹ is -NR¹⁰R¹¹.

In certain embodiments of Formula 3, R¹ is -NR¹²C(O)R¹³.

In certain embodiment, R² is H.

In some instances of Formula 3, the R¹ group is linked via a linker to an affinity agent (e.g., as described herein). In some cases, the linker includes a branched linker (e.g., as described herein). For example, in embodiments where R¹ is -NR¹⁰R¹¹ the R¹ group can be linked via a linker to an affinity agent through either the R¹⁰ group or the R¹¹ group. In other cases, where R¹ is -NR¹²C(O)R¹³, the R¹ group can be linked via a linker to an affinity agent through the R¹³ group.

In some instances of Formulae 1, 2 or 3, Q² is a ligand, as described according to the present disclosure.

In some instances of Formulae 1, 2 or 3, Q² is a label, as described according to the present disclosure. For example, the label can be a detectable label, such as a fluorophore, as described herein.

In some instances of Formulae 1, 2 or 3, Q² is a reactive group, as described according to the present disclosure.

In some instances of Formulae 1, 2 or 3, Q² is a second affinity agent, as described according to the present disclosure.

In some cases, a photoisomerizable group present in a conjugate of the present disclosure is an azobenzene compound as shown below:

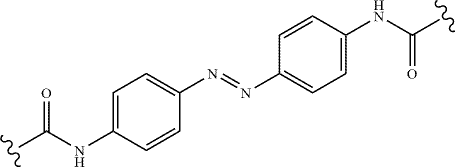

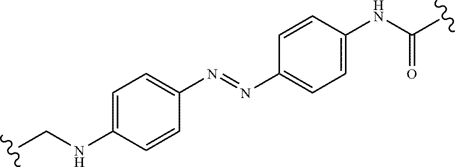

where the wavy lines indicate the attachment points to the rest of the conjugate. For instance, the wavy line on the left side of the azobenzene may indicate the attachment point to a linker (e.g., a branched linker as described herein) and the wavy line on the right side of the azobenzene may indicate the attachment point to a ligand as described herein.

In some cases, the photoisomerizable group is an azobenzene, such as an azobenzene photoisomerizable groups are found in WO 2019/060785, the disclosure of which is incorporated herein by reference in its entirety.

### Ligands

As used herein, the term “ligand” refers to a molecule (e.g., a small molecule, a peptide, or a protein) that binds to a polypeptide and effects a change in an activity of the polypeptide, and/or effects a change in conformation of the polypeptide, and/or affects binding of another polypeptide to the polypeptide, or affects the impact of another ligand on the polypeptide. Ligands include agonists, partial agonists, inverse agonists, antagonists, allosteric modulators, and blockers.

In some cases, the ligand is a naturally-occurring ligand. In other cases, the ligand is a synthetic ligand. In some cases, the ligand is an endogenous ligand. In some cases, the ligand is an agonist. In some cases, the ligand is an inverse agonist. In other cases, the ligand is a partial agonist. In other cases, the ligand is an antagonist. In other cases, the ligand is an allosteric modulator. In other cases, the ligand is a blocker. The term “antagonist” generally refers to an agent that binds to a ligand-binding polypeptide and inhibits the binding of the ligand-binding polypeptide. An “antagonist” may be an agent that binds to or near the orthosteric site (same site where an agonist binds) or an allosteric site but does not activate the ligand-binding polypeptide; instead, the antagonist generally excludes binding by an agonist or hinders activation by the agonist and thus prevents or hinders activation. An “allosteric modulator” may be an agent that binds to an allosteric site away from an orthosteric ligand binding site where binding of an allosteric ligand either decreases the sensitivity to or efficacy of an orthosteric ligand (negative allosteric modulator) or increases the sensitivity to or efficacy of an orthosteric ligand (positive allosteric modulator). The term “blocker” refers to an agent that acts directly on the active site, pore, or allosteric site. Ligands suitable for use herein bind reversibly to a ligand-binding site of a ligand-binding polypeptide.

The ligand is selected based in part on the target ligand-binding polypeptide, and the desired effect on the target ligand-binding polypeptide. For example, a ligand for a hormone-binding transcription factor will in some cases be a hormone, or a synthetic analog of the hormone, or a ligand that interferes with or modulates positively or negatively hormone binding or action. A ligand for a tetracycline transactivator will in some cases be tetracycline or a synthetic analog thereof. A ligand for an enzyme will in some cases be a synthetic agonist or antagonist of the enzyme. In some cases, a ligand will block the ligand-binding site. A ligand for an enzyme or ion channel will in some case be a blocker of the enzyme active site or ion channel pore. A ligand for a ligand-gated ion channel or a G protein coupled receptor or other membrane associated or soluble receptors will in some cases be a naturally-occurring ligand, or a synthetic version of the ligand, e.g., a synthetic analog of the ligand, or a ligand that interferes with or modulates positively or negatively the binding or action of that ligand.

In some cases, a ligand is a small molecule ligand. Small molecule ligands can have a molecular weight in a range of from about 50 daltons to about 3000 daltons, e.g., from about 50 daltons to about 75 daltons, from about 75 daltons to about 100 daltons, from about 100 daltons to about 250 daltons, from about 250 daltons to about 500 daltons, from about 500 daltons to about 750 daltons, from about 750 daltons to about 1000 daltons, from about 1000 daltons to about 1250 daltons, from about 1250 daltons to about 1500 daltons, from about 1500 daltons to about 2000 daltons, from about 2000 daltons to about 2500 daltons, or from about 2500 daltons to about 3000 daltons.

In other cases, a ligand is a peptide ligand. Peptide ligands can have a molecular weight in a range of from about 1 kDa to about 20 kDa, e.g., from about 1 kDa to about 2 kDa, from about 2 kDa to about 5 kDa, from about 5 kDa to about 7 kDa, from about 7 kDa to about 10 kDa, from about 10 kDa to about 12 kDa, from about 12 kDa to about 15 kDa, or from about 15 kDa to about 20 kDa. Peptide ligands can have a length of from 2 amino acids to 20 amino acids, e.g., a peptide ligand can have a length of 2, 3, 4, 5, 6, 7, 8, 9, 10, 11, 12, 13, 14, 15, 16, 17, 18, 19, or 20 amino acids. Peptide ligands can have a length of from 2 amino acids to 5 amino acids, from 5 amino acids to 10 amino acids, from 10 amino acids to 15 amino acids, or from 15 amino acids to 20 amino acids. Peptide ligands can be longer than 20 amino acids, e.g., up to 200 amino acids.

Suitable ligands include, but are not limited to, ligands that block or activate the function of a ligand-binding protein, where ligand-binding proteins include ion and macromolecule permeant channels; receptors (including, but not limited to, ionotropic receptors that bind transmitters; ionotropic receptors that bind hormones; metabotropic receptors and other G protein coupled receptors; receptor tyrosine kinases; growth factor receptors; and other membrane receptors that signal by binding to soluble or membrane-bound or extracellular small molecules or proteins); transporters (including but not limited to ion transporters, organic molecule transporters, peptide transporters, and protein transporters); enzymes (including but not limited to kinases; phosphatases; ubiquitin ligases; acetylases; oxo-reductases; lipases; enzymes that add lipid moieties to proteins or remove them; proteases; and enzymes that modify nucleic acids, including but not limited to ligases, helicases, topoisomerases, and telomerases); motor proteins (including kinesins, dyenins and other microtubule-based motors, myosins and other actin-based motors, DNA and RNA polymerases and other motors that track along polynucleotides); scaffolding proteins; adaptor proteins; cytoskeletal proteins; and other proteins that localize or organize protein domains and superstructures within cells.

Suitable ligands include, but are not limited to, ligands that function as general anesthetics; ligands that function as local anesthetics; ligands that function as analgesics; synthetic and semi-synthetic opioid analgesics (e.g., phenanthrenes, phenylheptylamines, phenylpiperidines, morphinans, and benzomorphans) where exemplary opioid analgesics include morphine, oxycodone, fentanyl, pentazocine, hydromorphone, meperidine, methadone, levorphanol, oxymorphone, levallorphan, codeine, dihydrocodeine, hydrocodone, propoxyphene, nalmefene, nalorphine, naloxone, naltrexone, buprenorphine, butorphanol, nalbuphine, and pentazocine; ionotropic glutamate receptor agonists and antagonists, e.g., N-methyl-D-aspartate (NMDA) receptor agonists, antagonists, and allosteric modulators, kainate (KA) receptor agonists and antagonists, and allosteric modulators, α-amino-3-hydroxy-5-methylisoxazole-4-propionic acid (AMPA) receptor agonists and antagonists and allosteric modulators, and metabotropic glutamate receptor agonists and antagonists and allosteric modulators; non-opioid analgesics, e.g., acetylsalicylic acid, choline magnesium trisalicylate, acetaminophen, ibuprofen, fenoprofen, diflusinal, and naproxen; muscarinic receptor agonists; muscarinic receptor antagonists; acetylcholine receptor agonists; acetylcholine receptor antagonists; serotonin receptor agonists; serotonin receptor antagonists; enzyme inhibitors; a benzodiazepine, e.g. chlordiazepoxide, clorazepate, diazepam, flurazepam, lorazepam, oxazepam, temazepam or triazolam; a barbiturate sedative, e.g. amobarbital, aprobarbital, butabarbital, butabital, mephobarbital, metharbital, methohexital, pentobarbital, phenobarbital, secobarbital, talbutal, theamylal, or thiopental; an H₁ antagonist having a sedative action, e.g. diphenhydramine, pyrilamine, promethazine, chlorpheniramine, or chlorcyclizine; an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, topiramate, neramexane, or perzinfotel; an alpha-adrenergic, e.g. doxazosin, tamsulosin, clonidine, guanfacine, dexmetatomidine, modafinil, phentolamine, terazasin, prazasin or 4-amino-6,7-dimethoxy-2-(5-methane-sulfonamido-1,2,3,4-tetrahydroisoquinol-2-yl)-5-(2-pyridyl) quinazoline; a tricyclic antidepressant, e.g. desipramine, imipramine, amitriptyline, or nortriptyline; an anticonvulsant, e.g. carbamazepine, lamotrigine, topiratmate, or valproate; a tachykinin (NK) antagonist, particularly an NK-3, NK-2 or NK-1 antagonist, e.g. (α-R,9R)-7-[3,5-bis(trifluoromethyl)benzyl]-8,9,10,1 1-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S); a muscarinic antagonist, e.g. oxybutynin, tolterodine, propiverine, tropsium chloride, darifenacin, solifenacin, temiverine, or ipratropium; a cyclooxygenase-2 (COX-2) selective inhibitor, e.g. celecoxib, rofecoxib, parecoxib, valdecoxib, deracoxib, etoricoxib, or lumiracoxib; a vanilloid receptor agonist (e.g. resinferatoxin) or antagonist (e.g. capsazepine); a beta-adrenergic such as propranolol; a 5-HT receptor agonist or antagonist, e.g., a 5-HT₁B/₁D agonist such as eletriptan, sumatriptan, naratriptan, zolmitriptan or rizatriptan; a 5-HT₂A receptor antagonist such as R( + )-α-(2,3-dimethoxy-phenyl)-1-[2-(4-fluorophenylethyl)]-4-piperidinemethanol (MDL-100907); and the like.

Suitable ligands for Na⁺ channels include, but are not limited to, lidocaine, novocaine, xylocaine, lignocaine, novocaine, carbocaine, etidocaine, procaine, prontocaine, prilocaine, bupivacaine, cinchocaine, mepivacaine, quinidine, flecainide, procaine, N-[[2′-(aminosulfonyl)biphenyl-4-yl]methyl]-N′-(2,2′-bithien-5-ylmethyl)succinamide (BPBTS), QX-314, saxitoxin, tetrodotoxin, and a type III conotoxin. Suitable ligands for Na⁺ channels also include, but are not limited to, tetrodotoxin, saxitoxin, guanidinium, polyamines (e.g. spermine, cadaverine, putrescine, µ-conotoxin, and δ-conotoxin.

Suitable ligands for K⁺ channels include, but are not limited to, quaternary ammonium (e.g., tetraethyl ammonium, tetrabutylammonium, tetrapentylammonium), 4-aminopyridine, sulfonylurea, Glibenclamide; Tolbutamide; Phentolamine, quinine, quinidine, peptide toxins (e.g., charybdotoxin, agitoxin-2, apamin, dendrotoxin, VSTX1, hanatoxin-1, hanatoxin-2, and Tityus toxin K-α.

Suitable ligands for CNG and HCN channels include, but are not limited to, 1-cis diltiazem and ZD7288. Suitable ligands for glycine receptors include, but are not limited to, strychnine and picrotoxin.

Suitable ligands for nicotinic acetylcholine receptors include, but are not limited to, (+)-tubocurarine, Methyllycaconitine, gallamine, Nicotine; Anatoxin A, epibatidine, ABT-94, Lophotoxin, Cytisine, Hexamethonium, Mecamylamine, and Dihydro-β-erythroidine. Suitable ligands for muscarinic acetylcholine receptors include, but are not limited to, a muscarinic acetylcholine receptor antagonist as described in U.S. Pat. No. 7,439,255; AF267B (see, e.g., U.S. Pat. No. 7,439,251); phenylpropargyloxy-1,2,5-thiadiazole-quinuclidine; carbachol; pirenzapine; migrastatin; a compound as described in U.S. Pat. No. 7,232,841; etc.

Suitable ligands for GABA receptors include, but are not limited to, Muscimol, THIP, Procabide, bicuculine, picrotoxin, gabazine, gabapentin, diazepam, clonazepam, flumazenil, a β-carboline carboxylate ethyl ester, baclofen, faclofen, and a barbiturate.

Many suitable ligands will be known to those skilled in the art; and the choice of ligand will depend, in part, on the target (e.g., receptor, ion channel, enzyme, etc.) to which the ligand binds.

In some cases, e.g., where the target ligand-binding polypeptide is a D1 dopamine receptor, the ligand is a D1 dopamine receptor agonist. Suitable D1 dopamine receptor agonists include dihydrexidine derivatives, benzazepine derivatives, and the like. Suitable D1 dopamine receptor agonists include, e.g., dopamine, 2-(N-phenethyl-N-propyl)-amino-5-hydroxytetralin (PPHT), dihydrexidine, A-86929, dinapsoline, dinoxyline, doxanthrine, SKF-81297, SKF-82958, SKF-38393, clozapine, fenoldopam, 6-Br-APB, stepholidine, A-68930, A-77636, CY-208,243, SKF-89145, SKF-89626, 7,8-dihydroxy-5-phenyl-octahydrobenzo[h]isoquinoline, apomorphine, pergolide, rotigotine, terguride, and cabergoline.

Dopamine has the following structure:

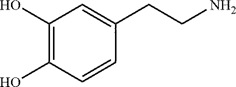 [SMILES: NCCc1ccc(O)c(O)c1]

Dihydrexidine has the following structure:

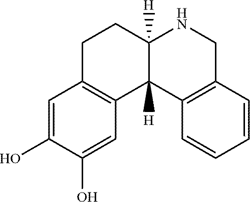 [SMILES: Oc1cc2c(cc1O)[C@H]1c3ccccc3CN[C@@H]1CC2]

In some cases, e.g., where the target ligand-binding polypeptide is a D1 dopamine receptor, the ligand is a positive allosteric modulator of the D1 dopamine receptor. See, e.g., Luderman et al. (2018) Mol. Pharmacol. 94:1197; and Meltzer et al. (2019) Behav. Brain Res. 361:139. Suitable D1 dopamine receptor positive allosteric modulators include, but are not limited to, MLS6585, MLS1082, and DETQ (2-(2,6-dichlorophenyl)-1-((1S,3R)-3-(hydroxymethyl)-5-(2-hydroxypropan-2-y1)-1-methyl-3,4-dihydroisoquinolin-2(1H)-y1)ethan-1-one).

MLS6585 has the following structure:

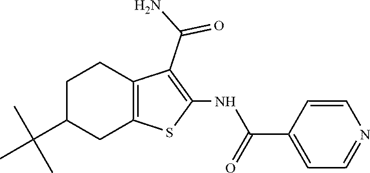 [SMILES: CC(C)(C)C1CCc2c(sc(NC(=O)c3ccncc3)c2C(N)=O)C1]

MLS1082 has the following structure:

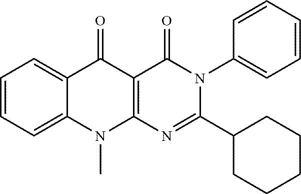 [SMILES: Cn1c2ccccc2c(=O)c2c(=O)n(-c3ccccc3)c(C3CCCCC3)nc21]

In some cases, where the target ligand-binding polypeptide is a D1 dopamine receptor, the ligand is a D1 receptor antagonist. In some cases, the D1 dopamine receptor antagonist comprises the structure of the PD-1 _{block} depicted in FIG. 23.

In some cases, e.g., where the target ligand-binding polypeptide is a D2 dopamine receptor, the ligand is a D2-like agonist such as quinpirole, rotigotine, B-HT920, bromocriptine, cabergoline, piribedil, pramipexole, or sumanirole. In some cases, a D2 agonist comprises the structure of the P-D2_{ago} depicted in FIG. 23.

In some cases, e.g., where the target ligand-binding polypeptide is a D2 dopamine receptor, the ligand is a D2 dopamine receptor antagonist, such as eticlopride; sulpiride; raclopride; L-741,626; domperidone; or an antipsychotic such as haloperidol, chlorpromazine, or clozapine. In some cases, a D2 antagonist comprises the structure of the P-D2_{block} depicted in FIG. 23.

In some cases, e.g., where the target ligand-binding polypeptide is a D2 dopamine receptor, the ligand is a D2 dopamine receptor-biased ligand. Examples of such ligands include dihydrexidine, aripiprazole, UNC9994, MLS1547. See, e.g., Agren et al. (2018) Int’l J. Neuropsychopharmacol. 21:1102.

UNC9994 has the following structure:

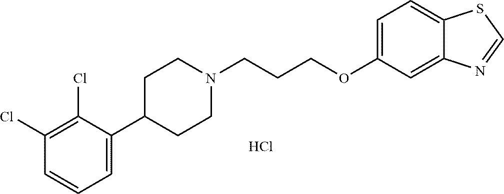 [SMILES: Clc1cccc(C2CCN(CCCOc3ccc4scnc4c3)CC2)c1Cl ; Cl]

MLS1547 has the following structure:

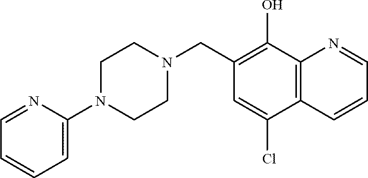 [SMILES: Oc1c(CN2CCN(c3ccccn3)CC2)cc(Cl)c2cccnc12]

### Linkers

Suitable linkers include, but are not limited to, a polycarbon chain; poly(ethylene glycol); a peptide; and the like. In some cases, the linker is a C₁-C₂₅ alkyl. In some cases, the linker is a substituted C₁-C₂₅ alkyl. In some cases, the linker is poly(ethylene glycol) (PEG), where the PEG comprises from 2 to 50 ethylene glycol monomers; e.g., the PEG comprises from 2 to 5, from 5 to 10, from 10 to 15, from 15 to 20, from 20 to 25, from 25 to 30, from 30 to 35, from 35 to 40, from 40 to 45, or from 45 to 50, ethylene glycol units.

In some cases, the linker is a peptide of from 2 amino acids to 50 amino acids; e.g., from 2 amino acids to 5 amino acids, from 5 amino acids to 10 amino acids, from 10 amino acids to 15 amino acids, from 15 amino acids to 20 amino acids, from 20 amino acids to 25 amino acids, from 25 amino acids to 30 amino acids, or from 30 amino acids to 50 amino acids. In some cases, the linker is a peptide of 2, 3, 4, 5, 6, 7, 8, 9, or 10 amino acids in length.

### Fusion Polypeptides

As noted above, a system of the present disclosure includes, in addition to a conjugate as described above, either: b1) a fusion polypeptide, or a recombinant expression vector comprising a nucleotide sequence encoding the fusion polypeptide, wherein the fusion polypeptide comprises: i) a self-labeling protein tag; ii) a peptide linker; and iii) a membrane-anchoring polypeptide; or b2) a fusion polypeptide, or a recombinant expression vector comprising a nucleotide sequence encoding the fusion polypeptide, wherein the fusion polypeptide comprises: i) a self-labeling protein tag; and ii) an antibody specific for the D1 dopamine receptor.

### Self-Labelling Polypeptides

Suitable self-labelling polypeptides (also referred to herein as “self-labelling protein tags”) include a SNAP polypeptide, a CLIP polypeptide, or a HALO polypeptide. Also suitable for use is a halo-based oligonucleotide binder (HOB) polypeptide. See, e.g., Kossman et al. (2016) Chembiochem. 17:1102. A HOB polypeptide binds chlorohexyl moieties. Also suitable for use is a trimethoprim (TMP) tag, an engineered form of E. coli dihydrofolate reductase (DHFR) that forms a non-covalent high-affinity complex with trimethoprim derivatives. See, e.g., Gallagher et al. (2009) ACS Chem. Biol. 4:547; and Jing and Cornish (2013) ACS Chem. Biol. 8:1704.

A SNAP polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

MDKDCEMKRTTLDSPLGKLELSGCEQGLHRIIFLGKGTSAADAVEVPAPAAVLGGPEPL
MQATAWLNAYFHQPEAIEEFPVPALHHPVFQQESFTRQVLWKLLKVVKFGEVISYSHLA
ALAGNPAATAAVKTALSGNPVPILIPCHRVVQGDLDVGGYEGGLAVKEWLLAHEGHRL
GKPGLG (SEQ ID NO:1). A SNAP polypeptide binds O⁶-benzylguanine (BG).

A CLIP polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

MDKDCEMKRTTLDSPLGKLELSGCEQGLHRIIFLGKGTSAADAVEVPAPAAVLGGPEPL
IQATAWLNAYFHQPEAIEEFPVPALHHPVFQQESFTRQVLWKLLKVVKFGEVISESHLA
ALVGNPAATAAVNTALDGNPVPILIPCHRVVQGDSDVGPYLGGLAVKEWLLAHEGHRL
GKPGLG (SEQ ID NO:2). A CLIP polypeptide can bind O²-benzylcytosine (BC).

A HALO polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

MAEIGTGFPFDPHYVEVLGERMHYVDVGPRDGTPVLFLHGNPTSSYVWRNIIPHVAPTH
RCIAPDLIGMGKSDKPDLGYFFDDHVRFMDAFIEALGLEEVVLVIHDWGSALGFHWAK
RNPERVKGIAFMEFIRPIPTWDEWPEFARETFQAFRTTDVGRKLIIDQNVFIEGTLPMGVV
RPLTEVEMDHYREPFLNPVDREPLWRFPNELPIAGEPANIVALVEEYMDWLHQSPVPKL
LFWGTPGVLIPPAEAARLAKSLPNCKAVDIGPGLNLLQEDNPDLIGSEIARWLSTLEISG
(SEQ ID NO:3). A HALO polypeptide binds chloroalkane.

### Membrane Anchors

Suitable membrane anchors include polypeptides that insert into a eukaryotic cell plasma membrane (e.g., a mammalian cell plasma membrane). Suitable membrane anchors include single-pass transmembrane polypeptides. The membrane anchor may span the entire plasma membrane, but need not do so. The membrane anchor can be any natural or artificial transmembrane domain may comprise a hydrophobic a-helix of about 20 amino acids. Prediction of transmembrane domains/segments may be made using publicly available prediction tools (e.g. TMHMM, Krogh et al. (2001) J. Molec. Biol. 305(3):567-580; and TMpred, Hofmann and Stoffel (1993) Biol. Chem. Hoppe-Seyler 347:166).

The transmembrane domain of any polypeptide can be used as the membrane anchor. Non-limiting examples include, e.g., the membrane anchor of glycophorin A (GPA), the membrane anchor of small integral membrane protein 1 (SMIM1), a platelet derived growth factor receptor (PDGFR) transmembrane domain, and the like.

### Endoplasmic Reticulum Export Signals

In some cases, a fusion polypeptide includes: i) a self-labelling polypeptide; ii) a peptide linker; iii) a membrane anchoring polypeptide; and iv) an endoplasmic reticulum (ER) export signal peptide.

Suitable ER export signal peptides include, e.g., VXXSL (SEQ ID NO:4) (where X is any amino acid) (e.g., VKESL (SEQ ID NO:5); VLGSL (SEQ ID NO:6); etc.); NANSFCYENEVALTSK (SEQ ID NO:7); FXYENE (SEQ ID NO:8) (where X is any amino acid), e.g., FCYENEV (SEQ ID NO:9); and the like. An ER export signal peptide can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

### Peptide Linkers

As noted above, in some cases, a fusion polypeptide includes: i) a self-labelling polypeptide; ii) a peptide linker; iii) a membrane anchoring polypeptide. In some cases, the peptide linker is a rigid linker, such as a peptide of the formula: [A(EAAAK)nA]x (SEQ ID NO: 10) (where n=2-4 and x=1 or 2). Another example of a suitable rigid linker is a peptide of the formula: (XP)n (SEQ ID NO: 11), where X is any amino acid (e.g., where X is Ala, Lys, or Glu), and where n is 1, 2, 3, 4, 5, 6, 7, 8, 9, or 10. In some cases, the peptide linker comprises the amino acid sequence (EAAAK)n (SEQ ID NO: 12), where n is an integer from 1 to 6.

### Antibody to D1 Dopamine Receptor

As noted above, in some cases, a fusion polypeptide includes: i) a self-labelling polypeptide; and ii) an antibody specific for a D1 dopamine receptor. In some cases, the anti-D1 dopamine receptor antibody is a nanobody. In some cases, the anti-D1 dopamine receptor antibody is a scFv.

### Nucleic Acids

As noted above, a system of the present disclosure can include a fusion polypeptide per se, as described above, or can include a nucleic acid (e.g., an expression vector) comprising a nucleotide sequence encoding the fusion polypeptide.

The nucleic acid can be an expression vector. The nucleotide sequence can be operably linked to a promoter. The expression vector can be, e.g., a recombinant viral expression vector (e.g., a recombinant adenovirus-associated virus vector; a recombinant retroviral vector; and the like). The nucleotide sequence encoding the fusion polypeptide can be operably linked to one or more transcriptional control elements (e.g., promoters; enhancers). For example, the nucleotide sequence encoding the fusion polypeptide can be operably linked to one or more transcriptional control elements that provide for preferential expression in a target cell type. As an example, the nucleotide sequence encoding the fusion polypeptide can be operably linked to one or more transcriptional control elements that provide for preferential expression in a D1 dopamine receptor. For example, the nucleotide sequence encoding the fusion polypeptide can be operably linked to D1 dopamine receptor promoter and enhancer elements.

### Conjugates

The present disclosure provides a conjugate of the present disclosure is a compound having the formula: (A)-X₁-(B)-X₂-(C), where:
A is an antibody specific for a D1 dopamine receptor;
B is a photoisomerizable group;
C is a ligand that binds to the D1 dopamine receptor and that functions as a D1 dopamine receptor agonist;
X₁, when present, is a linker; and
X₂, when present, is a linker.

### Affinity Agents

An affinity agent included in a conjugate of the present disclosure is an antibody specific for a D1 dopamine receptor. In some cases, the anti-D1 dopamine receptor antibody is a nanobody. In some cases, the anti-D1 dopamine receptor antibody is a scFv.

### Photoisomerizable Groups

Photoisomerizable groups are known in the art, and any known photoisomerizable group can be included in the photoisomerizable regulator present in a conjugate of the present disclosure. Suitable photoisomerizable groups include, but are not limited to, azobenzene, cyclic azobenzenes and azoheteroarenes and derivatives thereof; spiropyran and derivatives thereof; triphenyl methane and derivatives thereof; 4,5-epoxy-2-cyclopentene and derivatives thereof; fulgide and derivatives thereof; thioindigo and derivatives thereof; diarylethene and derivatives thereof; diallylethene and derivatives thereof; overcrowded alkenes and derivatives thereof; and anthracene and derivatives thereof. In some cases, a suitable photoisomerizable group is a photoisomerizable group as shown in the examples herein.

Suitable spiropyran derivatives include, but are not limited to, 1,3,3-trimethylindolinobenzopyrylospiran; 1,3,3-trimethylindolino-6′-nitrobenzopyrylospiran; 1,3,3-trimethylindolino-6′-bromobenzopyrylospiran; 1-n-decyl-3,3-dimethylindolino-6′-nitrobenzopyrylospiran; 1-n-octadecy-1-3,3-dimethylindolino-6′-nitrobenzopyrylospiran; 3′,3′-dimethyl-6-nitro-1′-[2-(phenylcarbamoyl)ethyl]spiro; [2H-1-benzopyran-2,2′-indoline]; 1,3,3-trimetnylindolino-8′-methoxybenzopyrylospiran; and 1,3,3-trimethylindolino-β-naphthopyrylospiran. Also suitable for use is a merocyanine form corresponding to spiropyran or a spiropyran derivative.

Suitable triphenylmethane derivatives include, but are not limited to, malachite green derivatives. specifically, there can be mentioned, for example, bis[dimethylamino)phenyl] phenylmethanol, bis[4-(diethylamino)phenyl]phenylmethanol, bis[4-(dibuthylamino)phenyl]phenylmethanol and bis[4-(diethylamino)phenyl]phenylmethane.

Suitable 4,5-epoxy-2-cyclopentene derivatives include, for example, 2,3-diphenyl-1-indenone oxide and 2′,3′-dimethyl-2,3-diphenyl-1-indenone oxide.

Suitable azobenzene compounds include, e.g., compounds having azobenzene residues crosslinked to a side chain, e.g., compounds in which 4-carboxyazobenzene is ester bonded to the hydroxyl group of polyvinyl alcohol or 4-carboxyazobenzene is amide bonded to the amino group of polyallylamine. Also suitable are azobenzene compounds having azobenzene residues in the main chain, for example, those formed by ester bonding bis(4-hydroxyphenyl)dimethylmethane (also referred to as bisphenol A) and 4,4′-dicarboxyazobenzene or by ester bonding ethylene glycol and 4,4′-dicarboxyazobenzene.

Suitable cyclic azobenzene and azoheteroarene compounds which can be adapted for use in the subject conjugates and photoisomerizable regulators include, but are not limited to, 11,12-dihydrodibenzo[c,g] [1,2]diazocine-5-oxide,

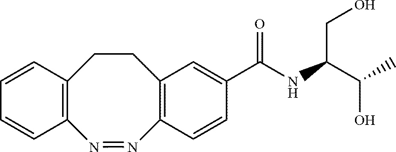 [SMILES: C[C@H](O)[C@H](CO)NC(=O)c1ccc2c(c1)CCc1ccccc1/N=N\2]

, heterodiazocines, such as those photoswitches described by Hammerich et al. J. Am. Chem. Soc., 2016, 138 (40), pp 13111-13114), and azoheteroarene photoswitches such as 3-pyrazoles (3 pzH or 3 pzMe), 5-pyrazoles (5 pzH or 5 pzMe), 3-pyrrroles (3 pyH or 3 pyMe), triazole and tetrazoles (tet or 3 tri) as describes by Calbo et al. J. Am. Chem. Soc., 2017, 139 (3), pp 1261-1274, the disclosure of which is herein incorporated by reference.

Suitable fulgide derivatives include, but are not limited to, isopropylidene fulgide and adamantylidene fulgide.

Suitable diallylethene derivatives include, for example, 1,2-dicyano-1,2-bis(2,3,5-trimethyl-4-thienyl)ethane; 2,3-bis(2,3,5-trimethyl-4-thiethyl) maleic anhydride; 1,2-dicyano-1,2-bis(2,3,5-trimethyl-4-selenyl)ethane; 2,3-bis(2,3,5-trimethyl-4-selenyl) maleic anhydride; and 1,2-dicyano-1,2-bis(2-methyl-3-N-methylindole)ethane.

Suitable diarylethene derivatives include but are not limited to, substituted perfluorocylopentene-bis-3-thienyls and bis-3-thienylmaleimides.

Suitable overcrowded alkenes include, but are not limited to, cis-2-nitro-7-(dimethylamino)-9-(2’,3′-dihydro-1’H-naphtho[2,1-b]thiopyran-1′-ylidene)-9H-thioxanthene and trans-dimethyl-[1-(2-nitro-thioxanthen-9-ylidene)-2,3 -dihydro-1 H-benzo [f] thiochromen-8-yl]amine. Overcrowded alkenes are described in the literature. See, e.g., terWiel et al. (2005) Org. Biomol. Chem. 3:28-30; and Geertsema et al. (1999) Agnew CHem. Int. Ed. Engl. 38:2738.

Other suitable photoisomerizable groups include, e.g., reactive groups commonly used in affinity labeling, including diazoketones, aryl azides, diazerenes, and benzophenones.

In some instances, the photoisomerizable group of the conjugate (e.g., as defined herein) is an azobenzene (e.g., an azobenzene photoswitch) of the following formula:

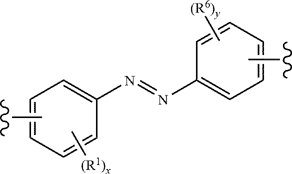

wherein:
R¹ and R⁶ are one or more optional substituents selected from hydrogen, C₁₋₁₀ alkyl, substituted C₁₋₁₀ alkyl, -NR¹⁰R¹¹, -NR¹²C(O)R¹³, -NR¹²C(O)OR¹³, -NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C4-10 cycloalkyl, C₄₋₁₀ cycloalkenyl, substituted C₄₋₁₀cycloalkenyl, cyano, halo, -OR¹⁰, -C(O)OR¹⁰, -SR¹⁰, -S(O)R¹⁰, -S(O)₂R¹⁰;
x is an integer from 1 to 5;
y is an integer from 1 to 5; and
wherein R¹⁰-R¹³ are as defined below,
or a pharmaceutically acceptable salt thereof.

In some cases, a photoisomerizable group present in a conjugate of the present disclosure is a compound of Formula 1:

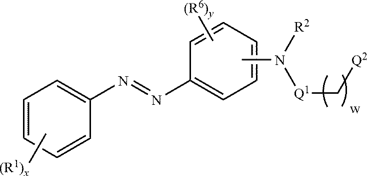

wherein:
Q¹ is —CH₂— or —C(═O)—;
Q² is a ligand (or a label or reactive group or second affinity agent), as described according to the present disclosure;
each R¹ is independently selected from hydrogen, C₁₋₁₀ alkyl, substituted C₁₋₁₀ alkyl, -NR¹⁰R¹¹, -NR¹²C(O)R¹³, -NR¹²C(O)OR¹³, -NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C4-10 cycloalkyl, C₄₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₁₀ cycloalkenyl;
each R⁶ is independently selected from hydrogen, C₁₋₁₀ alkyl, substituted C₁₋₁₀ alkyl, -NR¹⁰R¹¹, -NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₁₀ cycloalkyl, substituted C₄₋₁₀ cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
R¹⁰ and R¹¹ are each independently selected from hydrogen, C₁₋₁₀ alkyl, substituted C₁₋₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₁₀ cycloalkyl, substituted C₄₋₁₀ cycloalkyl, C₄₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₁₀ cycloalkyl, substituted C₄₋₁₀ cycloalkyl, C₄₁₀ cycloalkenyl, substituted C₄₋₁₀ cycloalkenyl, —CH₂—N(CH₂CH₃)₃⁺, and —CH₂—SO₃^{—}; or a pharmaceutically acceptable salt thereof.

In certain embodiments of Formula 1, Q¹ is —CH₂—. In certain embodiments of Formula 1, Q¹ is —C(═O)—.

In some instances of Formula 1, one of R¹ is linked via a linker to an affinity agent (e.g., as described herein). In some cases, the linker includes a branched linker (e.g., as described herein).

In some cases, a photoisomerizable group present in a conjugate of the present disclosure is a compound of Formula 2:

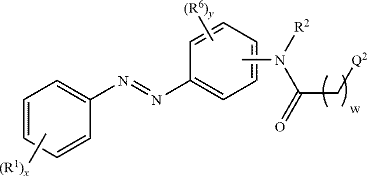

wherein
Q² is a ligand (or a label or reactive group or second affinity agent), as described according to the present disclosure;
each R¹ is independently selected from hydrogen, C₁₋₁₀ alkyl, substituted C₁₋₁₀ alkyl, -NR¹⁰R¹¹, -NR¹²C(O)R¹³, -NR¹²C(O)OR¹³, -NR¹²C(O)NR¹²R¹³, C₂₋₁₀alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₁₀ cycloalkenyl, cyano, halo, -OR¹⁰, -C(O)OR¹⁰, -SR¹⁰, -S(O)R¹⁰, -S(O)₂R₁₀;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₁₀ cycloalkenyl, and substituted C₄₁₀ cycloalkenyl;
each R⁶ is independently selected from hydrogen, C₁₋₁₀ alkyl, substituted C₁₋₁₀ alkyl, -NR¹⁰R11, -NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₁₀ cycloalkyl, substituted C₄₋₁₀ cycloalkyl, C₄₋₁₀ cycloalkenyl, substituted C₄₋₁₀ cycloalkenyl, cyano, halo, -OR¹⁰, -C(O)OR¹⁰, -SR¹⁰, -S(O)R¹⁰, -S(O)₂R¹⁰;
R¹⁰ and R¹¹ are each independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₁₀ cycloalkyl, substituted C₄₋₁₀ cycloalkyl, C₄₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C6-20 aryl, substituted C6-20 aryl, C₄₋₁₀ cycloalkyl, substituted C₄₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₁₀ cycloalkyl, C₄₋₁₀ cycloalkenyl, substituted C₄₋₁₀ cycloalkenyl, -CH₂-N(CH₂CH₃)³⁺, and —CH₂—SO₃^{—}; or a pharmaceutically acceptable salt thereof.

In some instances of Formula 2, one of the R¹ groups is linked via a linker to an affinity agent (e.g., as described herein). In some cases, the linker includes a branched linker (e.g., as described herein).

In some cases, a photoisomerizable group present in a conjugate of the present disclosure is a compound of Formula 3:

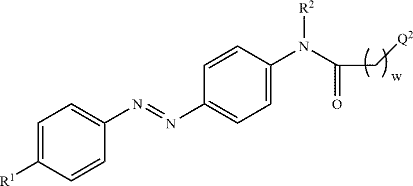

wherein:
Q² is a ligand (or a label or reactive group or second affinity agent), as described according to the present disclosure;
w is an integer from 1 to 10;
R¹ is selected from hydrogen, C₁₋₁₀ alkyl, -NR¹⁰R¹¹, -NR¹²C(O)R¹³, -NR¹²C(O)OR¹³ and -NR¹²C(O)NR¹²R¹³;
R² is hydrogen or C₁₋₁₀ alkyl;
R¹⁰ and R¹¹ are independently selected from hydrogen and C₁₋₁₀ alkyl;
R¹² is hydrogen or C₁₋₁₀ alkyl; and
R¹³ is selected from hydrogen, C₁₋₁₀ alkyl, C₁₋₈ alkenyl, C₆₋₁₀ aryl, and substituted C₁₋₁₀ alkyl,
or a pharmaceutically acceptable salt thereof.

In certain embodiments of Formula 3, R¹ is C₁₋₁₀ alkyl, such as C₁₋₈ alkyl, e.g., C₁₋₆ alkyl, C₁₋₅ alkyl or C₁₋₈ alkyl. In some embodiments of Formula 3, R¹ is C₁₋₈ alkyl.

In certain embodiments of Formula 3, R¹ is -NR¹⁰R¹¹.

In certain embodiments of Formula 3, R¹ is -NR¹²C(O)R¹³.

In certain embodiment, R² is H.

In some instances of Formula 3, the R¹ group is linked via a linker to an affinity agent (e.g., as described herein). In some cases, the linker includes a branched linker (e.g., as described herein). For example, in embodiments where R¹ is -NR¹⁰R¹¹, the R¹ group can be linked via a linker to an affinity agent through either the R¹⁰ group or the R¹¹ group. In other cases, where R¹ is -NR¹²C(O)R¹³, the R¹ group can be linked via a linker to an affinity agent through the R¹³ group.

In some instances of Formulae 1, 2 or 3, Q² is a ligand, as described according to the present disclosure.

In some instances of Formulae 1, 2 or 3, Q² is a label, as described according to the present disclosure. For example, the label can be a detectable label, such as a fluorophore, as described herein.

In some instances of Formulae 1, 2 or 3, Q² is a reactive group, as described according to the present disclosure.

In some instances of Formulae 1, 2 or 3, Q² is a second affinity agent, as described according to the present disclosure.

In some cases, a photoisomerizable group present in a conjugate of the present disclosure is an azobenzene compound as shown below:

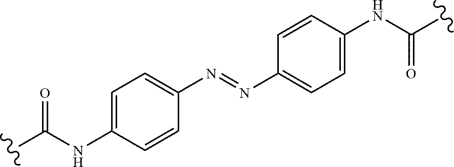

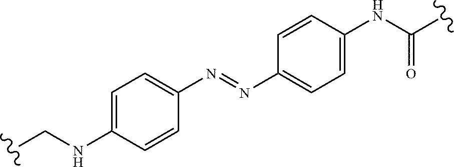

where the wavy lines indicate the attachment points to the rest of the conjugate. For instance, the wavy line on the left side of the azobenzene may indicate the attachment point to a linker (e.g., a branched linker as described herein) and the wavy line on the right side of theazobenzene may indicate the attachment point to a ligand as described herein.

In some cases, the photoisomerizable group is an azobenzene, such as an azobenzene photoisomerizable groups are found in WO 2019/060785, the disclosure of which is incorporated herein by reference in its entirety.

### Ligands

As noted above, in some cases, a ligand that is included in a conjugate of the present disclosure is a D1 dopamine receptor agonist. Suitable D1 dopamine receptor agonists include dihydrexidine derivatives, benzazepine derivatives, and the like. Suitable D1 dopamine receptor agonists include, e.g., dopamine, 2-(N-phenethyl-N-propyl)-amino-5-hydroxytetralin (PPHT), dihydrexidine, A-86929, dinapsoline, dinoxyline, doxanthrine, SKF-81297, SKF-82958, SKF-38393, clozapine, fenoldopam, 6-Br-APB, stepholidine, A-68930, A-77636, CY-208,243, SKF-89145, SKF-89626, 7,8-dihydroxy-5-phenyl-octahydrobenzo[h]isoquinoline, apomorphine, pergolide, rotigotine, terguride, and cabergoline.

As noted above, in some cases, a ligand that is included in a conjugate of the present disclosure is a D1 dopamine receptor allosteric modulator. In some cases, a ligand that is included in a conjugate of the present disclosure is a D2-like agonist. In some cases, a ligand that is included in a conjugate of the present disclosure is a D2 dopamine receptor antagonist. In some cases, a ligand that is included in a conjugate of the present disclosure is a D2-biased ligand.

### Linkers

Suitable linkers include, but are not limited to, a polycarbon chain; poly(ethylene glycol); a peptide; and the like. In some cases, the linker is a C₁-C₂₅ alkyl. In some cases, the linker is a substituted C₁-C₂₅ alkyl. In some cases, the linker is poly(ethylene glycol) (PEG), where the PEG comprises from 2 to 50 ethylene glycol monomers; e.g., the PEG comprises from 2 to 5, from 5 to 10, from 10 to 15, from 15 to 20, from 20 to 25, from 25 to 30, from 30 to 35, from 35 to 40, from 40 to 45, or from 45 to 50, ethylene glycol units.

In some cases, the linker is a peptide of from 2 amino acids to 50 amino acids; e.g., from 2 amino acids to 5 amino acids, from 5 amino acids to 10 amino acids, from 10 amino acids to 15 amino acids, from 15 amino acids to 20 amino acids, from 20 amino acids to 25 amino acids, from 25 amino acids to 30 amino acids, or from 30 amino acids to 50 amino acids. In some cases, the linker is a peptide of 2, 3, 4, 5, 6, 7, 8, 9, or 10 amino acids in length.

### Compositions

The present disclosure provides compositions, including pharmaceutical compositions, comprising a system or a conjugate of the present disclosure.

Compositions comprising a system of the present disclosure or a conjugate of the present disclosure can include one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-morpholino)ethanesulfonic acid (MES), 2-(N-morpholino)ethanesulfonic acid sodium salt (MES), 3-(N-Morpholino)propanesulfonic acid (MOPS), N-tris[hydroxymethyl]methyl-3-aminopropanesulfonic acid (TAPS), etc.; a solubilizing agent; a detergent, e.g., a non-ionic detergent such as Tween-20, Nonidet-P40, etc.; a protease inhibitor; and the like.

The present disclosure provides pharmaceutical compositions comprising a conjugate of the present disclosure. In some cases, the pharmaceutical composition is suitable for administering to an individual in need thereof. In some cases, the pharmaceutical composition is suitable for administering to an individual in need thereof, where the individual is a human.

A pharmaceutical composition comprising a system or a conjugate of the present disclosure may be administered to a patient alone, or in combination with other supplementary active agents. The pharmaceutical compositions may be manufactured using any of a variety of processes, including, without limitation, conventional mixing, dissolving, granulating, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

A pharmaceutical composition comprising a system or a conjugate of the present disclosure can optionally include a pharmaceutically acceptable carrier(s) that facilitate processing of an active ingredient into pharmaceutically acceptable compositions. As used herein, the term “pharmacologically acceptable carrier” refers to any carrier that has substantially no long-term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in “Pharmaceutical Dosage Forms and Drug Delivery Systems” (Howard C. Ansel et al., eds., Lippincott Williams & Wilkins Publishers, 7^{th} ed. 1999); “Remington: The Science and Practice of Pharmacy” (Alfonso R. Gennaro ed., Lippincott, Williams & Wilkins, 20^{th} 2000); “Goodman & Gilman’s The Pharmacological Basis of Therapeutics” Joel G. Hardman et al., eds., McGraw-Hill Professional, 10.sup.th ed. 2001); and “Handbook of Pharmaceutical Excipients” (Raymond C. Rowe et al., APhA Publications, 4^{th} edition 2003).

A subject pharmaceutical composition can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a subject pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a subject pharmaceutical composition.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) pH buffered solutions; (21) polyesters, polycarbonates and/or polyanhydrides; and (22) other non-toxic compatible substances employed in pharmaceutical formulations.

A conjugate of the present disclosure can be formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H.C. Ansel et al., eds., 7^{th} ed., Lippincott, Williams, & Wilkins; and Handbook of Pharmaceutical Excipients (2000) A.H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In a method of the present disclosure (described below), a conjugate of the present disclosure may be administered to the host using any convenient means capable of resulting in the desired reduction in disease condition or symptom. Thus, a conjugate of the present disclosure can be incorporated into a variety of formulations for therapeutic administration. More particularly, a conjugate of the present disclosure can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

A conjugate of the present disclosure can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

A system or a conjugate of the present disclosure can be administered as injectables. Injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

A system or a conjugate of the present disclosure can be formulated in a pharmaceutical composition together with a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

In some cases, a system or a conjugate of the present disclosure is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

### Methods

The present disclosure provides a method of modulating the activity of a target ligand-binding polypeptide (e.g., a D1 dopamine receptor; or a D2 dopamine receptor), the method comprising contacting a cell comprising the target ligand-binding polypeptide (e.g., the D1 dopamine receptor and/or the D2 dopamine receptor) with a system of the present disclosure or a conjugate of the present disclosure. The present disclosure provides a method of modulating the activity of a target ligand-binding polypeptide (e.g., a D1 dopamine receptor; or a D2 dopamine receptor), the method comprising: a) contacting a cell comprising the target ligand-binding polypeptide (e.g., the D1 dopamine receptor and/or the D2 dopamine receptor) with a system of the present disclosure or a conjugate of the present disclosure; and b) exposing the cell to light of a wavelength that isomerizes the photoisomerizable agent present in the conjugate. In some cases, the cell is present in an individual. For example, in some cases, the cell comprising the D1 dopamine receptor is in the dorsal striatum of an individual. In some cases, the cell is a direct pathway medium spiny neuron, and the ligand present in the conjugate is dopamine or a dopamine derivative or analog, where the ligand functions as a D1 dopamine receptor agonist.

The present disclosure provides a method of treating Parkinson’s disease in an individual, the method comprising administering a system of the present disclosure, or a conjugate of the present disclosure, into the dorsal striatum of the individual. The present disclosure provides a method of treating Parkinson’s disease in an individual, the method comprising: a) administering a system of the present disclosure, or a conjugate of the present disclosure, into the dorsal striatum of the individual; and b) exposing the dorsal striatum to light of a wavelength that isomerizes the photoisomerizable agent present in the conjugate, such that the ligand (dopamine or a dopamine derivative or analog, where the ligand functions as a D1 dopamine receptor agonist) binds to the D1 dopamine receptor. In some cases, the light is provided by an implantable light source. Administration of a system or a conjugate of the present disclosure can provide for an increase in motor function in an individual in need thereof.

Implantable light sources for use in delivering light to the brain of an individual are known in the art. See, e.g., Rossi et al. (2015) Front. Integr. Neurosci. 9:8; and McAlinden et al. (2019) Neutophotonics 6:035010. A suitable light source can include an implantable light-emitting diode (see Rossi et al. (2015) Front. Integr. Neurosci. 9:8). A suitable light source can include an implantable LED; and a control device for controlling light output by the LED.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A system comprising: a) a conjugate comprising: i) an affinity agent that forms a covalent bond with a self-labeling protein tag; ii) a linker; iii) a photoisomerizable group; and iv) a ligand that binds to a target ligand-binding polypeptide; and b) a fusion polypeptide, or a recombinant expression vector comprising a nucleotide sequence encoding the fusion polypeptide, wherein the fusion polypeptide comprises: i) a self-labeling protein tag; ii) a peptide linker; and iii) a membrane-anchoring polypeptide.
Aspect 2. The system of aspect 1, wherein the affinity agent comprises benzylguanine.
Aspect 3. The system of aspect 1, wherein the affinity agent comprises chloroalkane.
Aspect 4. The system of aspect 1, wherein the affinity agent comprises benzylcytosine.
Aspect 5. The system of any one of aspects 1-4, wherein the photoisomerizable group comprises a moiety selected from an azobenzene, a cyclic azobenzene, an azoheteroarene, a fulgide, a spiropyran, a triphenyl methane, a thioindigo, a diarylethene, and an overcrowded alkene.
Aspect 6. The system of any one of aspects 1-5, wherein the ligand is an agonist, an antagonist, an allosteric modulator, or a blocker.
Aspect 7. The system of any one of aspects 1-6, wherein the target ligand-binding polypeptide is selected from a transcription regulator, an ion channel, a cation channel, a ligand-gated ion channel, a voltage-gated ion channel, a quorum sensor, a pheromone receptor, a neurotransmitter receptor, or a G-protein-coupled receptor.
Aspect 8. The system of any one of aspects 1-7, wherein the target ligand-binding polypeptide is a D1 dopamine receptor, a D2 dopamine receptor, a glutamate receptor, a metabotropic glutamate receptor, an ionotropic glutamate receptor, an ionotropic nicotinic acetylcholine receptor, an ionotropic GABA-A receptor, a metabotropic GABA-B receptor, a metabotropic dopamine receptor, an ionotropic purinergic P2X receptor, a metabotropic purinergic P2Y receptor, a metabotropic serotonin receptor, an ionotropic serotonin receptor, an ionotropic glycine receptor, a cation channel, a potassium channel, a calcium channel, a sodium channel, a proton channel, an anion channel, or a chloride channel.
Aspect 9. The system of any one of aspects 1-8, wherein the photoisomerizable group comprises an azobenzene that isomerizes in response to visible light.
Aspect 10. The system of any one of aspects 1-9, wherein the target ligand-binding polypeptide is: a) a D1 dopamine receptor; or b) a D2 dopamine receptor.
Aspect 11. The system of aspect 10, wherein the ligand is dopamine or a dopamine derivative or analog that functions as a D1 dopamine receptor agonist.
Aspect 12. The system of aspect 10, wherein the ligand is a positive allosteric modulator of the D1 dopamine receptor.
Aspect 13. The system of any one of aspects 1-12, wherein the linker comprises poly(ethylene glycol).
Aspect 14. The system of any one of aspects 1-13, wherein the self-labeling protein tag comprises:
   a) an amino acid sequence having at least 80% amino acid sequence identity to the SNAP polypeptide amino acid sequence set forth in SEQ ID NO:1;
   b) an amino acid sequence having at least 80% amino acid sequence identity to the CLIP polypeptide amino acid sequence set forth in SEQ ID NO:2; or
   c) an amino acid sequence having at least 80% amino acid sequence identity to the HALO polypeptide amino acid sequence set forth in SEQ ID NO:3.
Aspect 15. The system of any one of aspects 1-14, wherein the fusion polypeptide comprises an endoplasmic reticulum (ER) export signal peptide.
Aspect 16. The system of any one of aspects 1-15, wherein the peptide linker comprises the amino acid sequence EAAAK (SEQ ID NO:13).
Aspect 17. The system of any one of aspects 1-16, wherein the nucleotide sequence encoding the fusion polypeptide is operably linked to a cell type-specific promoter.
Aspect 18. The system of aspect 16, wherein the promoter is a dopamine-1 receptor promoter.
Aspect 19. A system comprising: a) a conjugate comprising: i) an affinity agent that forms a covalent bond with a self-labeling protein tag; ii) a linker; iii) a photoisomerizable group; and iv) a ligand that binds to a D1 dopamine receptor; and b) a fusion polypeptide, or a recombinant expression vector comprising a nucleotide sequence encoding the fusion polypeptide, wherein the fusion polypeptide comprises: i) a self-labeling protein tag; and ii) an antibody specific for the D1 dopamine receptor.
Aspect 20. The system of aspect 19, wherein the affinity agent comprises benzylguanine.
Aspect 21. The system of aspect 19, wherein the affinity agent comprises chloroalkane.
Aspect 22. The system of aspect 19, wherein the affinity agent comprises benzylcytosine.
Aspect 23. The system of any one of aspects 19-22, wherein the photoisomerizable group comprises a moiety selected from an azobenzene, a cyclic azobenzene, an azoheteroarene, a fulgide, a spiropyran, a triphenyl methane, a thioindigo, a diarylethene, and an overcrowded alkene.
Aspect 24. The system of any one of aspects 19-23, where the antibody is a nanobody or a single-chain Fv.
Aspect 25. A conjugate comprising: a) an antibody specific for a D1 dopamine receptor; b) a photoisomerizable group; and c) a ligand that binds to the D1 dopamine receptor.
Aspect 26. The conjugate of aspect 25, where the antibody is a nanobody or a single-chain Fv.
Aspect 27. The conjugate of aspect 25 or aspect 26, wherein the photoisomerizable group comprises an azobenzene.
Aspect 28. A method of modulating the activity of a target ligand-binding polypeptide, the method comprising:
   a) contacting a cell comprising the target ligand-binding polypeptide with a system of any one of aspects 1-23 or a conjugate of any one of aspects 25-27; and
   b) exposing the cell to light of a wavelength that isomerizes the photoisomerizable agent present in the conjugate.
Aspect 29. The method of aspect 28, wherein the cell is in an individual.
Aspect 30. The method of aspect 28 or aspect 29, wherein the light is provided by an implantable light source.
Aspect 31. The method of any one of aspects 28-30, wherein the cell is a direct pathway medium spiny neuron, and wherein the ligand is dopamine or a dopamine derivative.
Aspect 32. A method of treating Parkinson’s disease in an individual, the method comprising administering the conjugate of any one of aspects 25-27 into the dorsal striatum of the individual.
Aspect 33. The method of aspect 32, comprising exposing the dorsal striatum to light of a wavelength that isomerizes the photoisomerizable agent present in the conjugate.
Aspect 34. The method of aspect 32 or aspect 33, wherein the light is provided by an implantable light source.
Aspect 35. A method of treating Parkinson’s disease in an individual, the method comprising administering the system of any one of aspects 1-24 into the dorsal striatum of the individual.
Aspect 36. The method of aspect 35, comprising exposing the dorsal striatum to light of a wavelength that isomerizes the photoisomerizable agent present in the conjugate.
Aspect 37. The method of aspect 35 or 36, wherein the light is provided by an implantable light source.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts,

temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Dopamine circuits control diverse behaviors and their dysregulation contributes to many disorders. The ability to understand and manipulate their function is hampered by the heterogenous nature of dopaminergic projections, the diversity of neurons that are regulated by dopamine, the varying distribution of dopamine receptors, and the complex dynamics of dopamine release. A solution to these challenges was developed in a generalizable photo-pharmacological approach called membrane-anchored photoswitchable orthogonal remotely tethered ligand (MP) that makes it possible to manipulate dopamine circuits with brain region, cell type, receptor subtype, and temporal specificity. By applying MP to dopamine receptors (MP-D), it was found that although dopaminergic input from the substantia nigra compacta has many targets in the dorsal striatum, its ability to trigger movement in mice is accounted for by dopamine D1 receptor activation in direct pathway-medium spiny neurons. The results provide a template for analyzing dopamine circuits and provide for treatment of dopamine-related disorders.

To examine D1R function in vivo, a photo-pharmacological strategy using an MP was implemented. A genetically-encoded membrane anchor captures and restricts the MP to a specific cell type. However, the MP has a unique photosensitive “PORTL” element that allows it to turn its target receptor on and off rapidly and reversibly in response to light, providing spatiotemporal control. A D1R/D5R selective-MP agonist was developed that is most potent on D1R (hence called MP-D1_{ago}). MP-D1_{ago} was targeted to dMSNs, where it would selectively activate D1R and avoid D5Rs in other striatal neurons. Photo-activation of MP-D1_{ago} in the dMSNs of the dStr, but not the ventral striatum’s nucleus accumbens (NAc), promoted movement initiation. Strikingly, this effect was as potent as optogenetic stimulation of dopaminergic nerve terminals that project to the dStr from the substantia nigra compacta (SNc). The MP method is applicable to other DARs and to neuronal receptors in general, providing unparalleled insight into neural circuit function and neuromodulation.

### Materials and Methods

### Chemistry

All reactions were carried out with magnetic stirring, and, if moisture- or air- sensitive, under nitrogen or argon atmosphere using standard Schlenk techniques in oven-dried glassware (140° C. oven temperature). External bath temperatures were used to record all reaction temperatures. Low temperature reactions were carried out in a Dewar vessel filled with distilled water/ice (0° C.). High temperature reactions were conducted using a heated silicon oil bath in reaction vessels equipped with a reflux condenser or in a sealed flask. Tetrahydrofuran (THF) was distilled over sodium and benzophenone prior to use. Dichloromethane (CH₂Cl₂), triethylamine (Et₃N) and diisopropylethylamine (DIPEA) were distilled over calcium hydride under a nitrogen atmosphere. All other solvents were purchased from Acros Organics as ‘extra dry’ reagents. All other reagents with a purity > 95% were obtained from commercial sources (Sigma Aldrich, Acros, Alfa Aesar, Base Click and others) and used without further purification.

Flash column chromatography was carried out with Merck silica gel 60 (0.040-0.063 mm). Analytical thin layer chromatography (TLC) was carried out using Merck silica gel 60 F254 glass-backed plates and visualized under UV light at 254 nm. Staining was performed with ceric ammonium molybdate (CAM) or by oxidative staining with an aqueous basic potassium permanganate (KMnO₄) solution and subsequent heating. HPLC was performed with HPLC grade solvents and deionized H₂O that was purified on a TKA MicroPure H2O purification system. All solvents were degassed with helium gas prior to use. Unless noticed otherwise, all experiments were carried out at room temperature.

NMR spectra (¹H NMR and ¹³C NMR) were recorded in deuterated chloroform (CDCl₃) or deuterated methanol (d₄-MeOH) on a Bruker Avance III HD 400 MHz spectrometer equipped with a CryoProbe™, a Varian VXR400 S spectrometer, a Bruker AMX600 spectrometer or a Bruker Avance III HD 800 MHz spectrometer equipped with a CryoProbe™ and are reported as follows: chemical shift δ in ppm (multiplicity, coupling constant J in Hz, number of protons) for ¹H NMR spectra and chemical shift δ in ppm for ¹³C NMR spectra. Multiplicities are abbreviated as follows: s = singlet, d = doublet, t = triplet, q = quartet, quint = quintet, br = broad, m = multiplet, or combinations thereof. Residual solvent peaks of CDCl₃ (δH = 7.26 ppm, δC = 77.16 ppm) and d₄-MeOH (δH = 3.31 ppm, δC = 49.00 ppm) were used as an internal reference. NMR spectra were assigned using information ascertained from COSY, HMBC, HSQC and NOESY experiments.

High resolution mass spectra (HRMS) were recorded on a Varian MAT CH7A or a Varian MAT 711 MS instrument by electron impact (EI) or electrospray ionization (ESI) techniques at the Department of Chemistry, Ludwig-Maximilians-University Munich. Infrared spectra (IR) were recorded from 4000 cm⁻¹ to 600 cm⁻¹ on a PERKIN ELMER Spectrum BX II, FT-IR instrument. For detection, a SMITHS DETECTION DuraSamplIR II Diamond ATR sensor was used. Samples were prepared as a neat film or a thin powder layer. IR data in frequency of absorption (cm⁻¹) is reported as follows: w = weak, m = medium, s = strong, br = broad or combinations thereof.

### Synthesis

All yields are isolated unless otherwise specified.

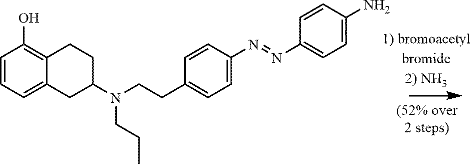

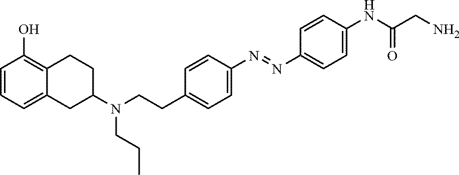 [SMILES: CCCN(CCc1ccc(/N=N/c2ccc(NC(=O)CN)cc2)cc1)C1CCc2c(O)cccc2C1]

Amine 2: Bromoacetylbromide (0.06 mL, 0.699 mmol) was added dropwise to an ice cooled solution of phenol 1¹³ (214 mg, 0.499 mmol) and DIPEA (0.12 mL, 0.699 mmol) in THF (8 mL) and the mixture was stirred for 10 min at that temperature and for 8 h at room temperature. The reaction was diluted with EtOAc and washed with saturated aqueous NaHCO₃ and brine. The organic phase was dried over MgSO₄ and concentrated under reduced pressure. The resulting residue was redissolved in THF (11 mL) and cooled to 0° C. Ammonia (7 M in MeOH, 4.00 mL, 28.0 mmol) was added and the mixture was stirred at room temperature overnight. The reaction was diluted with H₂O and extracted with EtOAc. The combined organic phases were washed with brine, dried over MgSO₄ and concentrated under reduced pressure. Purification of the resulting residue by flash column chromatography (MeOH:CH₂Cl₂:NH₃ (aq.) 4:96:1-7:93:1, R_{f} (MeOH:CH₂C1₂:NH₃ (aq.) 8:92:1) = 0.6) gave amine 2 (125 mg, 0.257 mmol, 52% over 2 steps) as an orange solid).

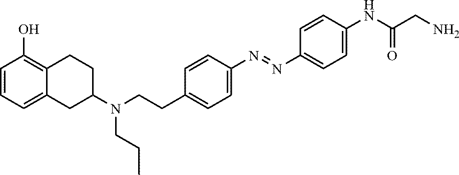 [SMILES: CCCN(CCc1ccc(/N=N/c2ccc(NC(=O)CN)cc2)cc1)C1CCc2c(O)cccc2C1]

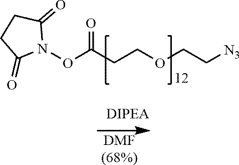

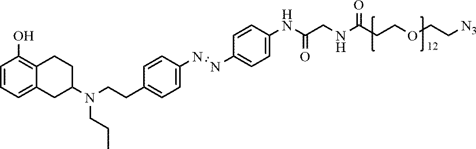

Azide 3: DIPEA (0.03 mL, 0.16 mmol) was added to a stirred solution of amine 2 (39.0 mg, 0.08 mmol) and Azido-(PEG)₁₂-NHS-ester (60.0 mg, 0.08 mmol) in DMF (0.5 mL) and the resulting solution was stirred at room temperature overnight. The reaction was diluted with EtOAc and washed with brine. The organic phase was dried over MgSO₄ and concentrated under reduced pressure. Purification of the resulting residue by reverse phase column chromatography (MeCN:HC1 (aq. 0.1%) (0:100-40:60) gave azide 3 (60 mg, 0.054 mmol, 68%) as an orange solid.

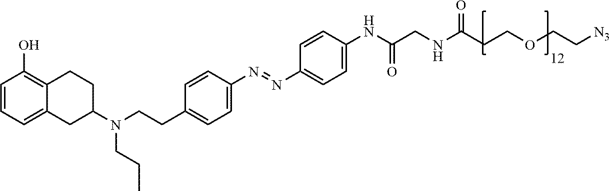

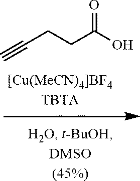

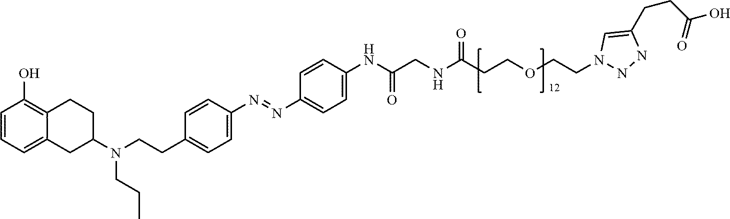

Acid 4: A solution of [Cu(MeCN)₄]PF₆ (12.4 mg, 0.033 mmol) in degassed t-BuOH/H₂O (2:1 3 mL) was added slowly to a solution of azide 3 (35 mg, 0.031 mmol), 4-pentynoic acid (6.2 mg, 0.063 mmol) and TBTA (17.7 mg, 0.033 mmol) in degassed t-BuOH/H₂O (1:1, 3 mL) and degassed DMSO (1 mL) at 0° C. and the mixture was stirred at room temperature for 24 h. The reaction was diluted with EtOAc and washed with brine. The organic phase was dried over MgSO₄ and concentrated under reduced pressure. Purification of the resulting residue by reverse phase column chromatography (MeCN:HCl (aq. 0.1%), 0:100-40:60) gave acid 4 (17 mg, 0.014 mmol, 45%) as an orange solid.

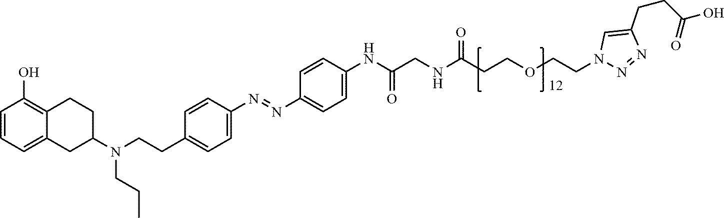

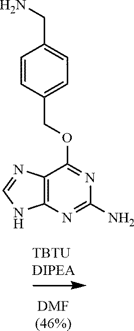

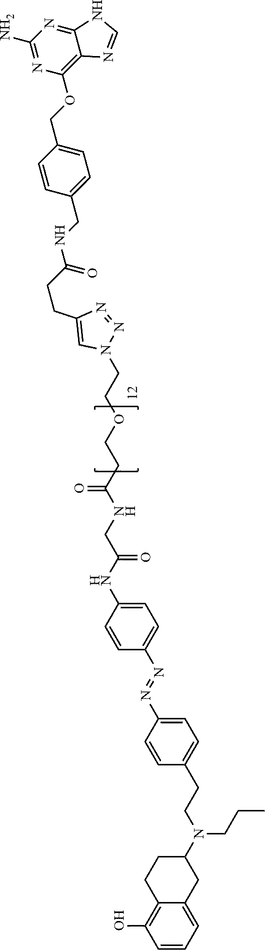

P-D1_{ago} (6): DIPEA (0.02 mL, 115 µmol) was added to a solution of acid 4 (9.0 mg, 7.40 µmol), amine 5¹(4.0 mg, 14.9 µmol) and TBTU (5.2 mg, 16.0 µmol) in DMF (0.7 mL) and the mixture was stirred at room temperature overnight. Purification by reverse phase HPLC (MeCN:H₂O:HCOOH 10:90:0.1-100:0:0.1) gave P-D1_{ago} (6) (5.0 mg, 3.40 µmol, 46%) as an orange solid.

### Structural Modeling

A homology model of D1R was generated using Bioluminate (Schrodinger, Inc)¹. The model was based on β2-adrenergic receptor (β2AR) because of the high relative degree of homology between D1R and β2AR. The amino acid sequence of D1R was aligned to β2AR in BLAST² and the TM segments were then structurally aligned with the crystal structure of β2AR bound to the inverse agonist carazolol (pdb: 2RH1). The three extracellular loops of D1R were refined using extended sampling. For docking DA into D1R, DA was prepared using LigPrep (Schrodinger, Inc) and docked with extra precision (XP) using Glide³. The hydroxyl groups of the three TM5 serines (Ser194^{5.42}, Ser195^{5.43} and Ser199^{5.46}) in D1R that contribute to the OBS were allowed to rotate during the docking procedure. As expected, the protonated amine and hydroxyls of DA were oriented towards Asp100^{3.32} in TM3 and the TM5 serines, respectively.

To dock the P-D1_{ago} analog PEG₄-azobenzene-PPHT in the D1R model, the protonated ligand was first prepared in ChemDraw (PerkinElmer) and Chimera (developed by the Resource for Biocomputing, Visualization, and Informatics at the University of California, San Francisco and supported by NIGMS P41-GM103311). It was then docked in the D1R model using Autodock Vina⁴. The following orthosteric binding site residues in D1R⁵ were allowed to rotate during the docking procedure: Asp100^{3.32}, Ile101^{3.33}, Ser104^{3.36}, Ser194^{5.42}, Ser195^{5.43}, Ser199^{5.46}, Trp282^{6.48}, Phe285^{6.51}, Phe286^{6.52}, Asn289^{6.55}, Val314^{7.39}, and Trp318^{7.43}.

To measure the distance from the benzylguanine binding site in the membrane-anchor (M) to the ligand binding site in D1R or mGluR5, a model of the M was made by placing the C-terminus of SNAP-tag (pdb: 3L00) flush with the N-terminus of a single-pass transmembrane segment (pdb: 2K1A). The M was then positioned adjacent to mGluR5 or D1R. The benzylguanine binding site in the M was oriented facing towards the receptor ligand binding site. To measure the lengths of P-D1_{ago} or BGAG, their extended states were first prepared in ChemDraw and end-to-end distances for each chemical component were measured in Chimera.

All molecular representations were prepared using Chimera.

### Molecular Biology and Heterologous Expression

All constructs were cloned into mammalian expression vectors. For the receptor mediated-GIRK activation assay, HEK293T cells were seeded onto 18 mm coverslips and transiently transfected overnight with Lipofectamine 2000 and the following constructs: a receptor, a variant of the membrane-anchor (M; 0.7 µg), GIRK1(F137S) (0.7 µg), and tdTomato (0.2 µg). The following receptors were tested: D1R (0.525 µg), opto-D1R (0.7 µg), D3R (0.2 µg), D5R (0.35 µg), A1R (0.7 µg), CB1R (0.7 µg), M1R (0.7 µg), M4R (0.7 µg), MOR (0.7 µg), mGluR1 (0.7 µg), and GABA_{B}R (0.7 µg of each subunit, B1 and B2). Some of these receptors do not couple to GIRK channels in HEK293T cells unless coexpressed with certain Gα subunits. D1R, D5R, or opto-D1R were cotransfected with Gαᵢₛ₁₃ (0.35 µg), D3R with Gα_{oA} (0.7 µg), and M1R or mGluR1 with Gα_{iq5}. Transfected cells were used for electrophysiology, imaging, or flow cytometry experiments.

For ligand gated-ion channel (LGIC) electrophysiology, HEK293T cells were seeded onto 18 mm coverslips and transiently transfected for 48 hours with Lipofectamine 2000 and the following constructs: MA_{EAAAK:ERE} (SEQ ID NO:17) (0.7 µg), tdTomato (0.2 µg), and one of the following: a non-desensitizing mutant of AMPAR GluR_{A1} (L497Y; 1.4 µg), the kainite receptor Glu_{K2} (0.7 µg), or GABA_{A}R_{α1β2γ2} (0.2 µg α1: 0.2 µg β2: 0.7 µg y2).

### Confocal Imaging and Flow Cytometric Analysis of Cultured Cells

Untransfected or membrane-anchor (M) expressing HEK293T cells were labeled with 1.5 µM BG-TMR or BG-Alexa647 (NEB) for 20-30 minutes in the dark at 37° C. and 5% CO2 in a standard extracellular solution. Cells were then (i) mounted on an upright, scanning confocal microscope (Zeiss LSM 780) and imaged with a 20x objective or (ii) washed in DPBS and measured by flow cytometry (BD LSR II).

### Electrophysiology

HEK293T cells were sparsely seeded and maintained in DMEM (Invitrogen) with 10% fetal bovine serum on poly-L-lysine-coated coverslips at 37° C. and 5% CO₂. HEK293T cells were voltage clamped in whole-cell configuration 16-48 hours after transfection. For GIRK experiments, the extracellular solution contained 120 mM KCl, 25 mM NaCl, 10 mM HEPES, 2 mM CaCl₂, and 1 mM MgCl₂, pH 7.4. opto-D1R cells were preincubated for 45 minutes with and patched in the presence of 1 µM 11-cis-retinal. For LGIC experiments, the extracellular solution contained 135 mM NaCl, 5.4 mM KCl, 10 mM HEPES, 2 mM CaCl₂, and 1 mM MgCh, pH 7.4. Glu_{K2} electrophysiology was performed in the presence of 0.3 mg/mL concanavalin A to prevent desensitization. Glass pipettes with a resistance of 3-7 MΩ were filled with intracellular solution containing 120 mM Gluconic acid 8-lactone, 15 mM CsCl, 10 mM BAPTA, 10 mM HEPES, 1 mM CaCl₂, 3 MgCl₂, 3 mM MgATP, pH 7.2. Cells were voltage clamped to -60 or -80 mV using an Axopatch 200A (Molecular Devices) amplifier.

To conjugate P-D1_{ago} to variants of the membrane-anchor (M), cells were incubated with varying concentrations of P-D1_{ago} for up to 60 minutes in the dark at 37° C. in standard extracellular buffers. For all experiments, compounds were applied using a gravity-driven perfusion system and illumination was applied to the entire field of view using a Polychrome V monochromator (TILL Photonics) through a 20x objective (maximum 0.05 mW/mm² at 370 nm and 0.5 mW/mm² at 460 nm or 473 nm) or a DG4 (Sutter) through a 20x objective (maximum 0.06 mW/mm² at 370 nm and 0.6 mW/mm² at 445 nm). pClamp software was used for both data acquisition and control of illumination.

The selection criteria for electrophysiological experiments are that a cell (i) expresses the fluorescent protein transfection marker, and (ii) responds to agonist, indicating the presence of either receptor and GIRK or an LGIC. Cells were not excluded unless the recording was of poor quality (e.g., unstable baseline).

### Animal Model

The following mouse lines were used for the experiments: D1-Cre (GENSAT, stock number: 017264-UCD, strain code: Tg(Drd1-cre)EY262Gsat/Mmucd) and DAT::IRES-Cre (Jackson Laboratory, stock number: 006660, strain code: B6.SJL-Slc6a3tm1.1(cre)Bkmn/J). The mice were 20-30 grams and 8-20 weeks old. Males and females were counterbalanced across conditions with no effects of sex observed. Mice were maintained on a 12:12 light cycle (lights on at 07:00). All procedures complied with the animal care standards set forth by the National Institutes of Health and were approved by University of California Berkeley’s Administrative Panel on Laboratory Animal Care.

### Light Intensity Calibration in Brain

To calibrate the intensity of light applied to the brain for in vivo behavioral experiments with MP-D1_{ago}, a protocol similar that described previously for measuring blue light transmission through rodent cortex was followed. Because light transmission depends on wavelength and brain area, the protocol was adjusted to measure the transmission of UV and blue light through striatal tissue. Mice were deeply anaesthetized with pentobarbital (200 mg/kg i.p.; Vortech) and then decapitated. Intracardial perfusion was not performed to avoid removing blood from the brain. Striatal tissue was excised and immediately placed in ice-cold artificial cerebrospinal fluid (ACSF) containing 50 mM sucrose, 125 mM NaCl, 25 mM NaHCO₃, 2.5 mM KCl, 1.25 mM NaH₂PO₄, 0.1 mM CaCl₂, 4.9 mM MgCl₂, and 2.5 mM glucose (oxygenated with 95% O₂:5% CO₂). The tissue was then placed in an ACSF-filled petri dish directly above a power meter (Thorlabs) with a ~200 µM pinhole. Using a micromanipulator (Sutter), the tip of a fiber (200 µM, 0.22 NA) emitting 375 nm or 450 nm light was placed directly above the power meter pinhole and then progressively lowered in 200 µM steps through air or striatal tissue. Power intensity values of each wavelength at each depth were used to calculate % transmission, % initial power density, and light intensity at each depth.

### AAVs

The adeno-associated virus (AAV) encoding M_{EAAAK}:_{ERE} (SEQ ID NO:17) and mVenus or mVenus alone were made. These cDNAs were inserted into an established Cre-dependent double-floxed inverted orientation (DIO) viral cassette under control of the CAG promoter and then packaged in the AAV5 capsid. The purified vectors contained 10¹³⁻¹⁴ viral genomes/ml. The AAV encoding ChR2, AAV-EF1a-DIO-hChR2(H134R)-eYFP, was obtained from the UNC vector core.

### Stereotaxic Surgeries

All stereotaxic injections were performed under general ketamine-dexmedetomidine anesthesia using a stereotaxic instrument (Kopf Instruments, Model 1900). For AAV injections, 400 nL of concentrated viral solution was injected into either the dStr (bregma: 0.5 mm, lateral: ±1.9 mm, ventral: -3.0 mm), the NAc (bregma: 1.4 mm, lateral: ±1.0 mm, ventral: -4.1 mm), or the SNc (bregma: 3.3 mm, lateral: ±1.3 mm, ventral: -4.1 mm) using a syringe pump (Harvard Apparatus) at 150 nL/minute. The injection needle was withdrawn 10 minutes after the end of each infusion. In some cases, immediately after viral injection mice were implanted with a bilateral guide cannula 0.5 mm dorsal to the injection site in the dStr or NAc. After injecting the ChR2 AAV into the SNc, a guide cannula was implanted in the dStr (bregma: 0.5 mm, lateral: ±1.9 mm, ventral: -2.5 mm). One layer of adhesive cement (C&B Metabond; Parkell) followed by cranioplastic cement (Dental cement) was used to secure the cannula to the skull. The incision was closed with tissue adhesive (Vetbond; 3M). The animals were kept on a heating pad until they recovered from anesthesia. Experiments were performed 4-12 weeks after stereotactic injection. Injection sites and cannula placements were confirmed in all animals by preparing coronal or sagittal sections (50 µm) of the brain. Although optical fiber placements varied slightly from mouse to mouse, behavioral data from all mice were included in the study.

### Open Field Test

The open field test was conducted to measure locomotor ability. Mice were placed in a custom-made white polycarbonate open field chamber (20 x 20 x 20 inches). Their movement was recorded with a video camera (Logitech) at ~20-30 Hz and analyzed using video-tracking software (Biobserve). Mice were subjected to one or more of the following prior to or during the behavioral test: (i) intraperitoneal injection, (ii) striatal infusion using a combination of infusion cannulas and a syringe pump (200 nL/minute; Harvard Apparatus), and (iii) light stimulation using optical cannulas (200 µM, 0.22 NA). See figure legends for specifics on the dosing and light intensities used for each experiment. In all cases, mice were habituated to these interventions for one to two days prior to the test day.

### Movement Analysis

The sub-second velocity traces recorded using Biobserve were smoothed in MATLAB using a first order Butterworth filter (normalized cutoff frequency ωᵤ = 0.02). Using original code written in MATLAB, a velocity threshold was manually set to distinguish movements from tracking artifacts, as verified by visual inspection of each movie. Movement bouts were counted when the velocity surpassed this threshold and lasted for at least 0.3 seconds. Bouts were considered finished after the velocity remained below the manually defined threshold for 0.1 seconds. The percent time moving was calculated as the time that velocity was above the movement threshold divided by the time of the entire measurement. Movement initiation frequency was calculated as the number of times velocity surpassed the movement threshold per second. Movement bout duration was calculated by averaging the duration of all movement bouts that occurred during the entire measurement. Max velocity was calculated by averaging the maximum velocity of all movement bouts that occurred during the entire measurement.

### Histology and Confocal Microscopy

In some cases, AAV-injected mice received a dStr-infusion of 100 µM SNAP-Surface Alexa Fluor 647 (400 nL per hemisphere; NEB) followed by intracardial perfusion 24-48 hours later. Otherwise, immunofluorescence was performed as described previously. Marcellino et al. (2012) Synapse 66:196; Dodson et al. (2016) Proc. Natl. Acad. Sci. USA 113:E2180. Briefly, after intracardial perfusion with 4% paraformaldehyde in PBS, pH 7.4, the brains were fixed overnight, placed in PBS with 30% sucrose for at least 24 hours, and then sliced into 50 µm coronal sections with a vibratome (Leica). The following primary antibodies were used: rabbit anti-HA tag (1:500; Cell Signaling), rat anti-D1R (1:500; Sigma-Aldrich), chicken anti-GFP/mVenus (1:1000; Abcam). The following secondary antibodies were used: goat anti-rabbit IgG-Alexa Flour 647 (1:750; Thermo Fisher Scientific), goat anti-rat IgG-Alexa 546 (1:750; Thermo Fisher Scientific), goat anti-chicken IgG-Alexa Flour 488 (1:1000; Thermo Fisher Scientific). Image acquisition was performed with a Zeiss LSM780 laser scanning confocal microscope using a 20x objective and on a Zeiss AxioImager M1 upright widefield fluorescence/differential interference contrast microscope with charge-coupled device camera using 5x objectives. Confocal images were analyzed using Zen (Zeiss) and ImageJ. Sections were identified using landmarks and neuroanatomical nomenclature¹².

### Statistics and Data Analysis

Data were analyzed using GraphPad Prism (GraphPad), Clampfit (Axon instruments), Origin (OriginLab), MATLAB, or ImageJ software. For dose-response curves, data were normalized to vehicle (0%) and DA (100%) and nonlinear regression analysis was performed using the sigmoidal dose-response function in GraphPad Prism. Statistical analyses were performed using GraphPad Prism. All values reported are mean ± S.E.M.

### References for Materials and Methods

1. Zhu, K. et al. Antibody structure determination using a combination of homology modeling, energy-based refinement, and loop prediction. Proteins 82, 1646-1655 (2014).
2. Coordinators, N.R. Database Resources of the National Center for Biotechnology Information. Nucleic acids research 45, D12-D17 (2017).
3. Friesner, R.A. et al. Glide: a new approach for rapid, accurate docking and scoring. 1. Method and assessment of docking accuracy. Journal of medicinal chemistry 47, 1739-1749 (2004).
4. Trott, O. & Olson, A.J. Software News and Update AutoDock Vina: Improving the Speed and Accuracy of Docking with a New Scoring Function, Efficient Optimization, and Multithreading. J Comput Chem 31, 455-461 (2010).
5. Michino, M. et al. What can crystal structures of aminergic receptors tell us about designing subtype-selective ligands? Pharmacological reviews 67, 198-213 (2015).
6. Aravanis, A.M. et al. An optical neural interface: in vivo control of rodent motor cortex with integrated fiberoptic and optogenetic technology. Journal of neural engineering 4, S143-S156 (2007).
7. Yizhar, O., Fenno, L.E., Davidson, T.J., Mogri, M. & Deisseroth, K. Optogenetics in Neural Systems. Neuron 71, 9-34 (2011).
8. Al-Juboori, S.I. et al. Light Scattering Properties Vary across Different Regions of the Adult Mouse Brain. PloS one 8 (2013).
9. Lammel, S. et al. Unique properties of mesoprefrontal neurons within a dual mesocorticolimbic dopamine system. Neuron 57, 760-773 (2008).
10. Lammel, S. et al. Input-specific control of reward and aversion in the ventral tegmental area. Nature 491, 212-217 (2012).
11. Lammel, S. et al. Diversity of Transgenic Mouse Models for Selective Targeting of Midbrain Dopamine Neurons. Neuron 85, 429-438 (2015).
12. Franklin, K.B.J. & Paxinos, G. Paxinos and Franklin’s The mouse brain in stereotaxic coordinates. (2013).

### Results

### Design of a D1R MP

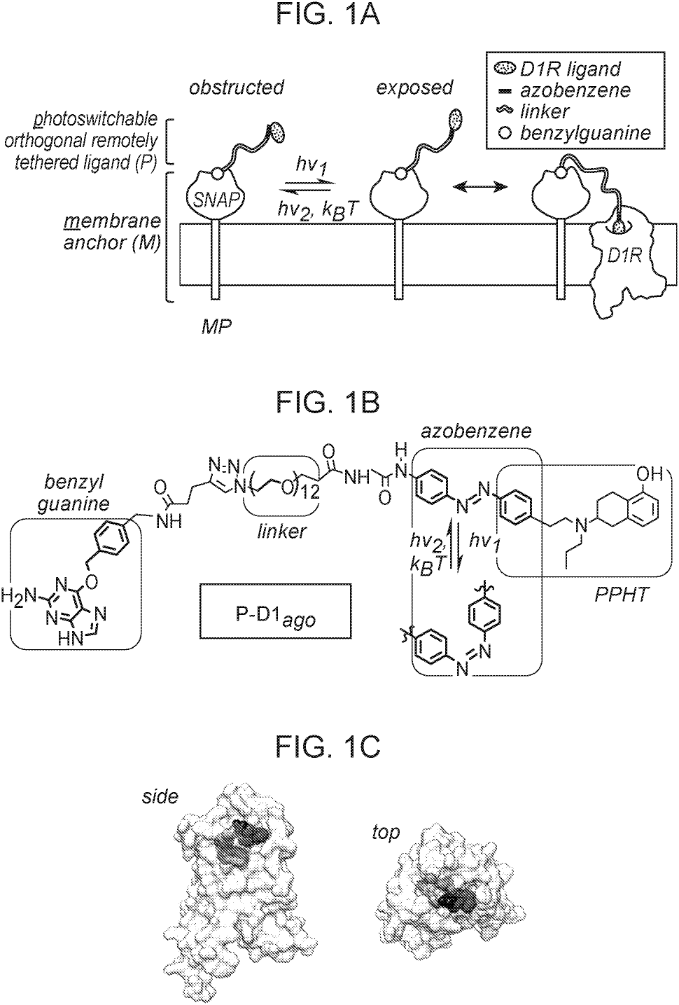

The MP is composed of four chemical components: a receptor ligand at one end, a protein-tag reactive moiety at the other end, an adjustable chemical linker in between, and an azobenzene that is flush to or merged with the receptor ligand. The azobenzene photoisomerizes between its trans and cis configurations in milliseconds (FIG. 1A), rapidly switching the receptor ligand between two states: one that is exposed and can bind its receptor and one that is obstructed by the azobenzene and so cannot bind. The MP is restricted to the membranes of cells that express a genetically-encoded protein-tag fused to a single-pass transmembrane segment (membrane anchor or M). In this instance, benzylguanine on the MP binds covalently and selectively to a SNAP-tag on the M. The chemical linker between azobenzene and benzylguanine is long enough to allow the MP to reach from its anchor point in the M to its binding site in the receptor.

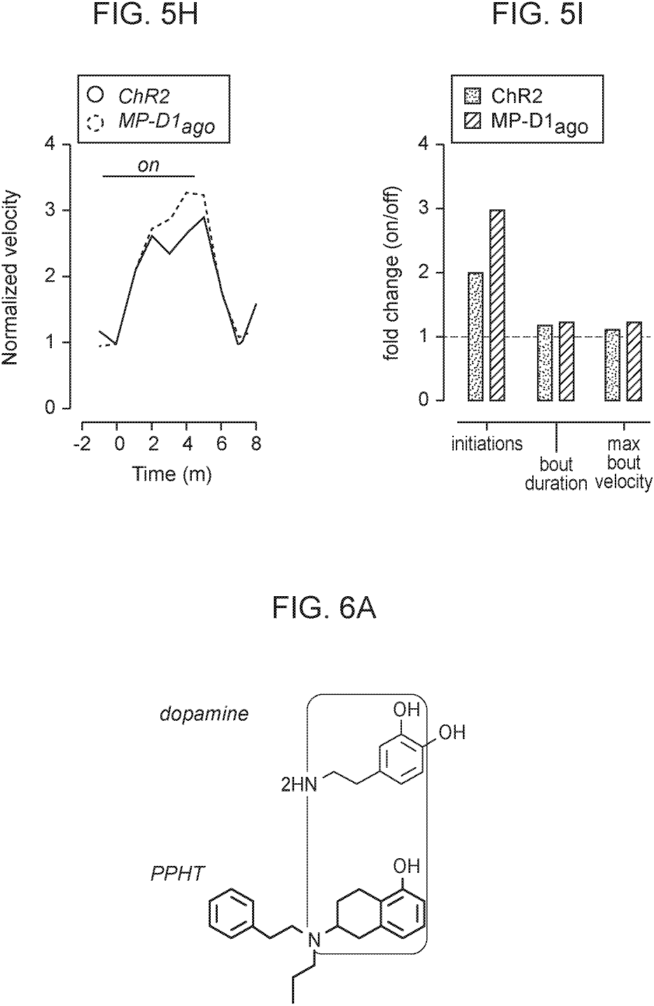

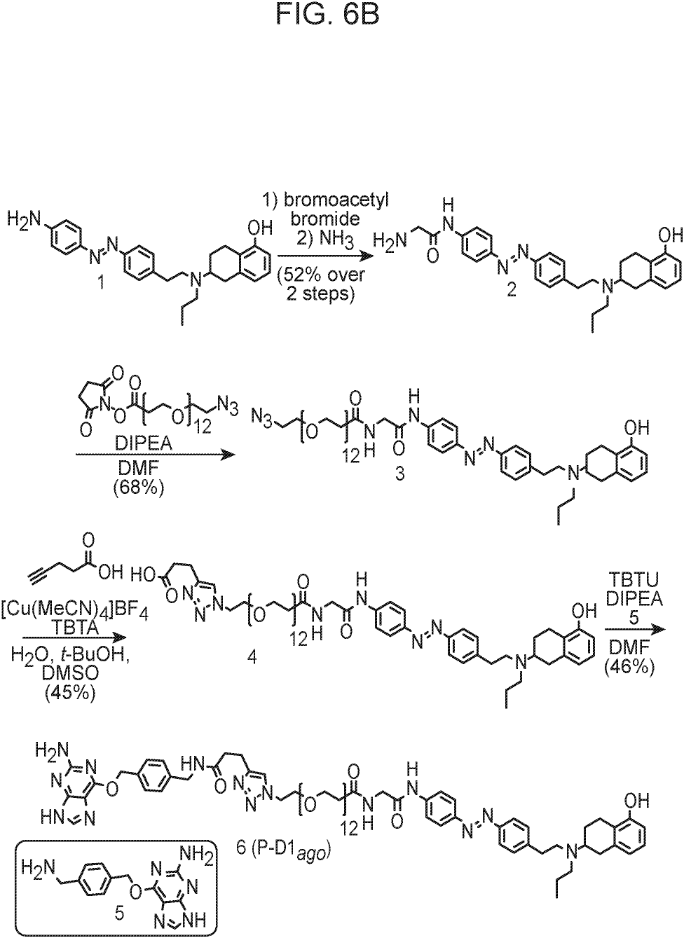

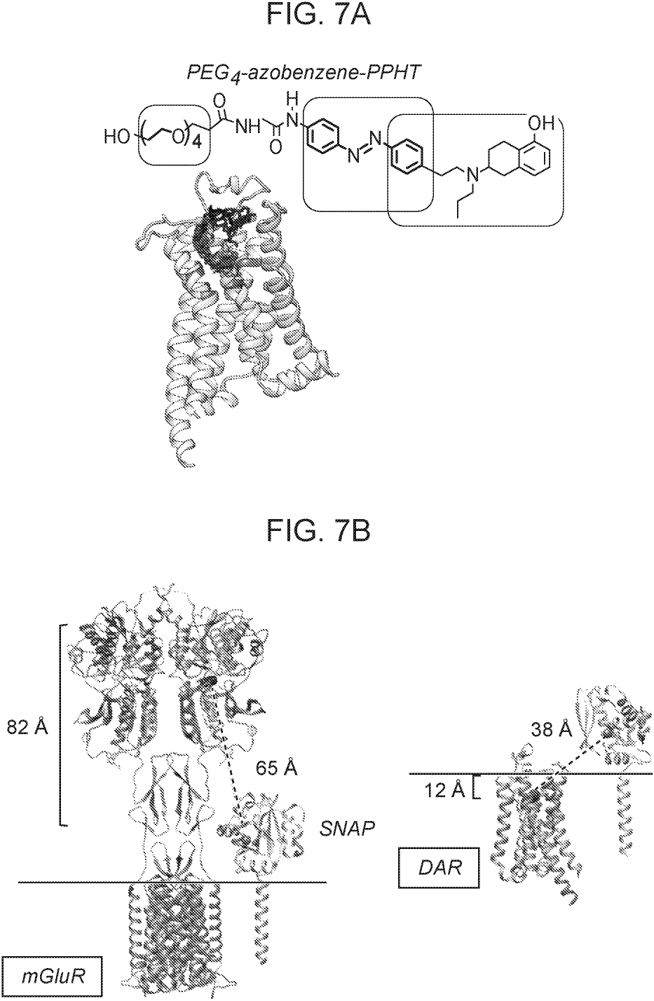

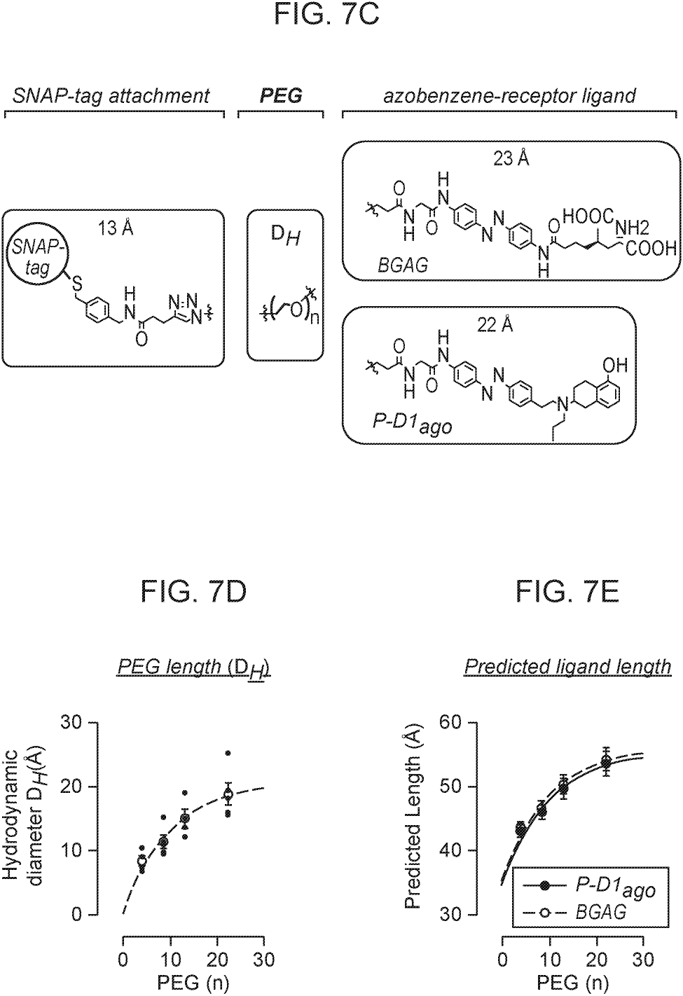

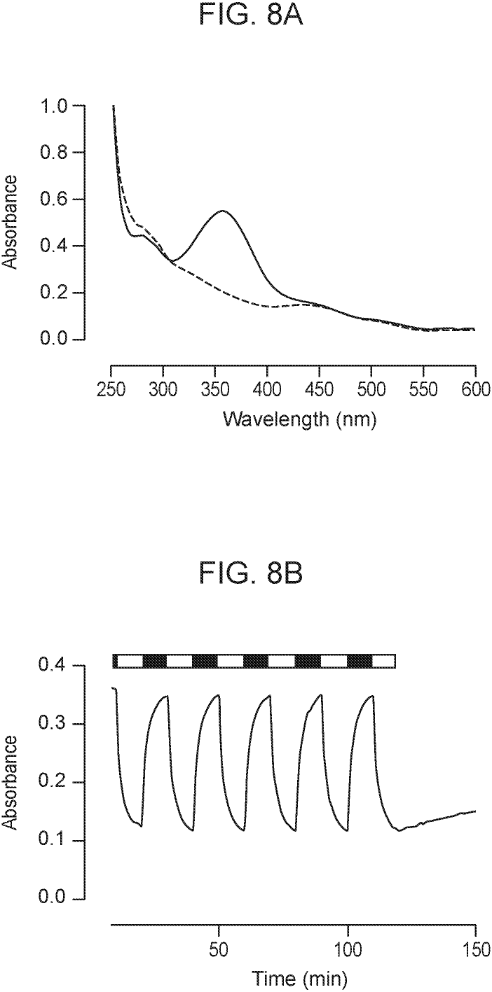

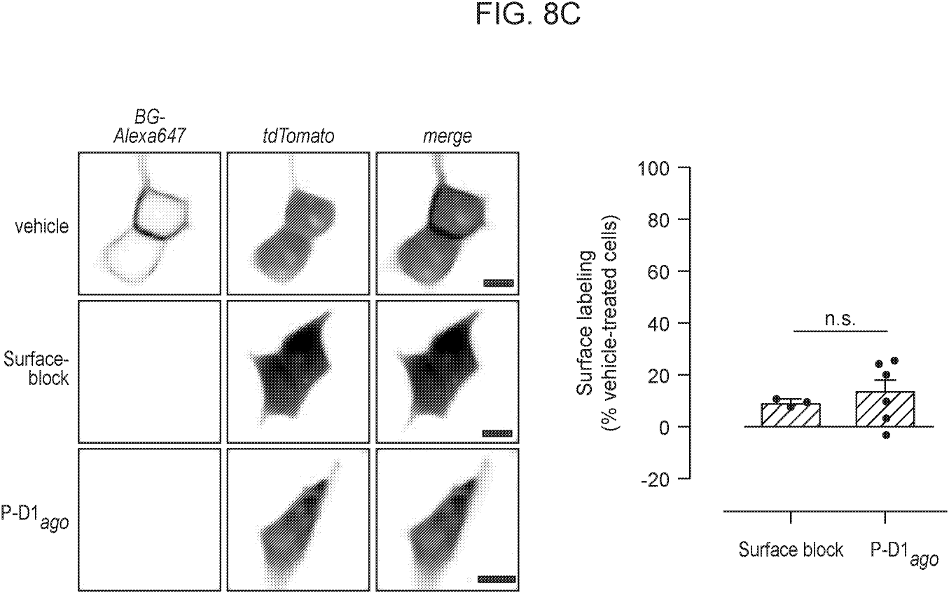

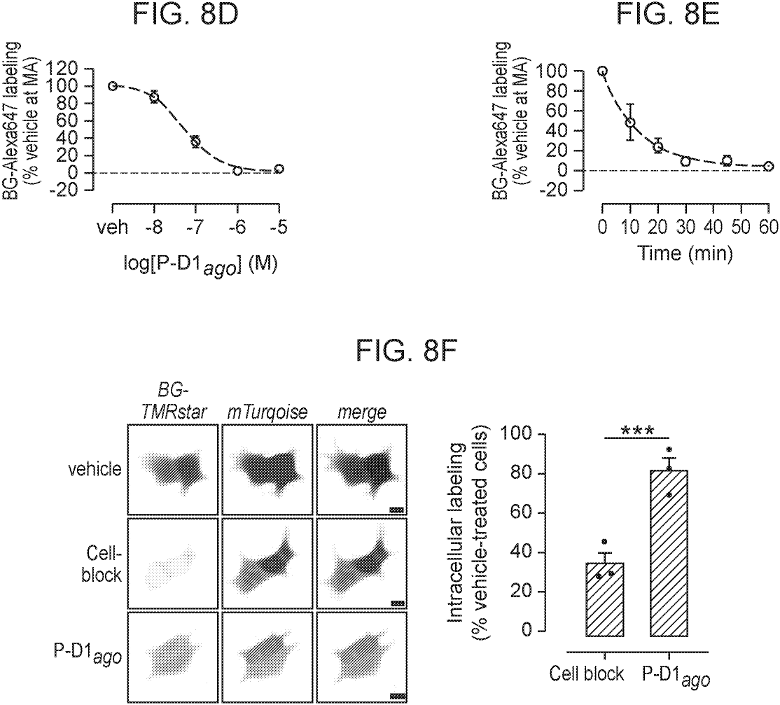

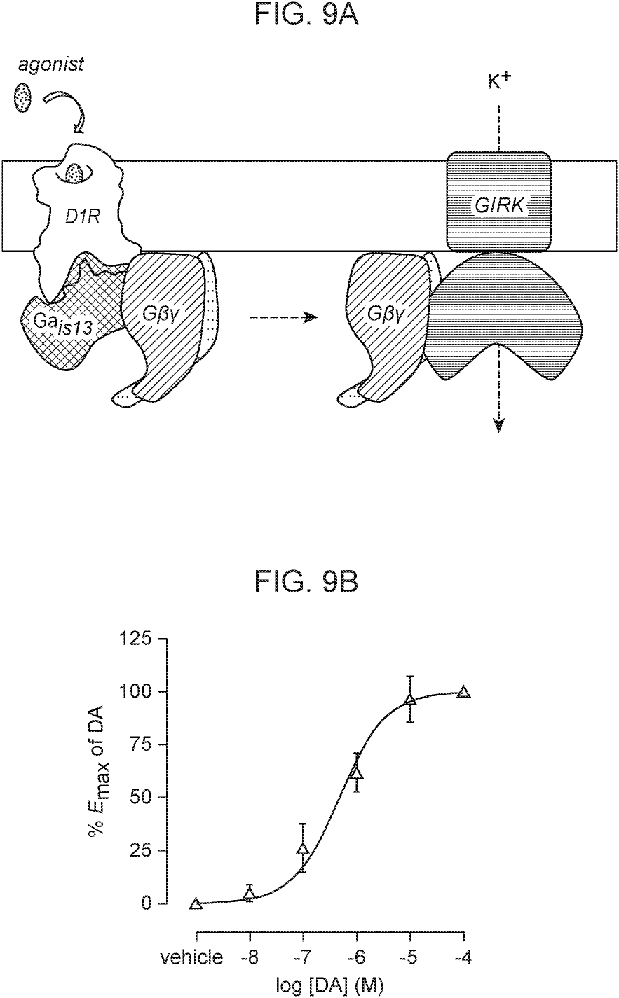

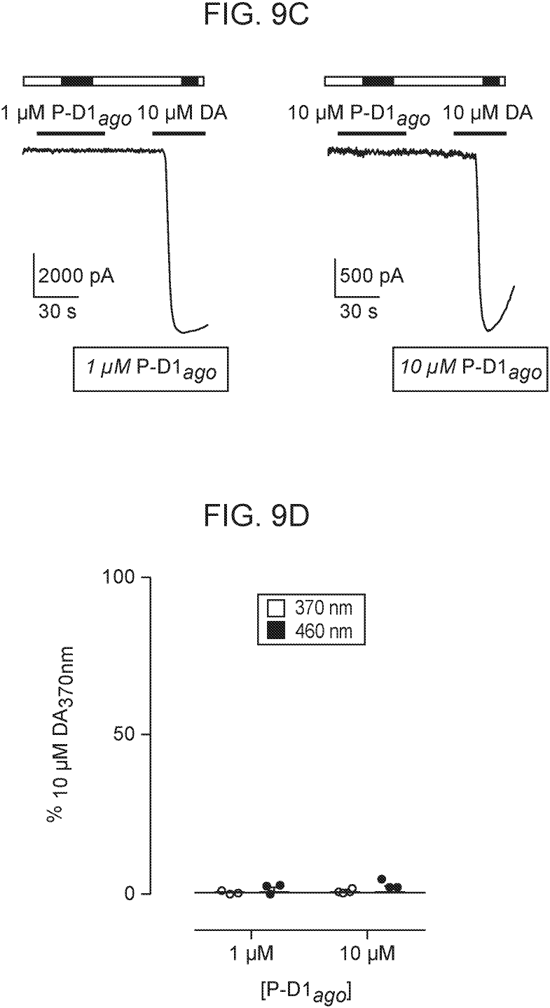

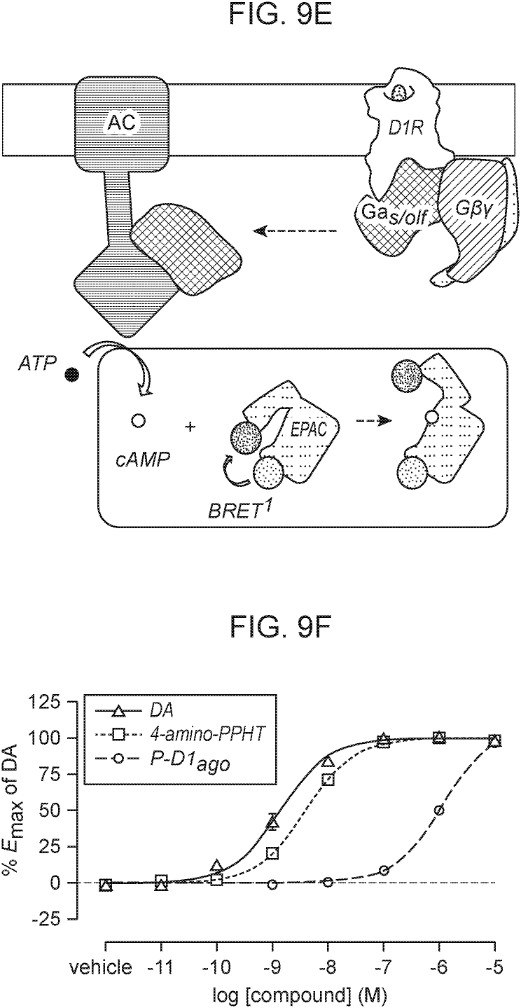

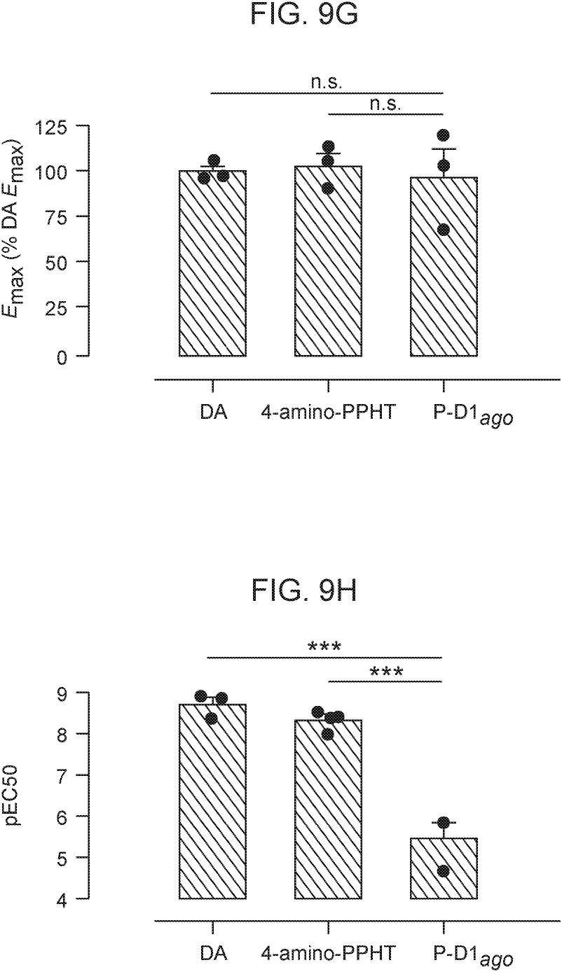

To develop a DIR MP, a metabolically stable analog of DA, 2-(N-phenethyl-N-propyl)-amino-5-hydroxytetralin (PPHT; FIG. 6A), was used as a parent molecule. It was previously shown that PPHT can tolerate the integration of azobenzene at its alkylbenzene. Donthamsetti, P.C. et al. (2017) Journal of the American Chemical Society 139, 18522-18535.When tethered directly to a cysteine introduced adjacent to the DA binding site of D1R via a maleimide group, azobenzene-PPHT binds the receptor in its trans state but not its cis state. Benzylguanine-azobenzene-PPHT (FIG. 1B; and FIG. 6B) was synthesized as a photoswitchable D1 agonist (P-D1_{ago}). Molecular modeling was used to optimize the positioning (FIG. 7A) and length (FIGS. 7B-7E) of the polyethylene glycol (PEG) linker between the azobenzene-PPHT ligand and the benzylguanine attachment site. P-D1ago switches from its trans-isomer to its cis-isomer under 370 nm (UV) light and back to trans-isomer under 460 nm (blue) light (FIGS. 8A and 8B). P-Dl_{ago} binds efficiently to the M on the cell surface (FIGS. 8C-8F) and is a low potency full agonist of D1R in its untethered form (FIGS. 9A-9H).

FIGS. 1A-1C. Design of a D1R MP. (A) Schematic representation of a D1R MP. The MP consists of D1R ligand that is tethered to the plasma membrane via membrane anchor (MA) consisting of SNAP-tag and transmembrane segment. The MP rapidly switches between its trans- and cis- isomers in response to specific wavelengths of light (hvi and hv₂), or slowly from the cis- to trans-isomer in the dark via thermal relaxation (kbT). The MP is designed to bind D1R only in the photoisomeric state that the receptor ligand is exposed. (B) The chemical structure of the D1R photoswitch benzylguanine-azobenzene-PPHT, a photoswitchable D1R agonist (P-D1_{ago}). Azobenzene (maroon) was integrated into the D1R agonist PPHT (orange) and is separated from benzylguanine (blue) via a 12-repeat polyethylene glycol (PEG) linker (green). (C) Model of a fragment of P-D1ago (trans-PEG₄-azobenzene-PPHT) docked in the dopamine (DA) binding site of D1R viewed from either the side (upper panel) or the top (bottom panel). Note that the linker (green) exits the pore that leads to the ligand binding site within the transmembrane domain of D1R.

FIGS. 6A-6B. Chemical synthesis of P-D1_{ago}. (A) Alignment of the dopamine (DA) with the synthetic DAR agonist PPHT, a rigidified aminotetralin analogue of DA that contains only one hydroxyl as well as N-propyl and N-phenylethyl groups that enhance metabolic stability in vivo and affinity toward DARs. (B) Synthesis scheme for P-D1_{ago}.

FIGS. 7A-7E. Design of P-D1_{ago}. (A) P-D1ago consists of azobenzene-PPHT conjugated to benzylguanine via a polyethylene glycol (PEG) chemical linker. Docking studies were used to provide insight into where the PEG linker should be attached to azobenzene. Multiple poses of a fragment of trans-P-D1_{ago} (PEG₄-azobenzene-PPHT) are shown docked in the dopamine (DA) binding site of a D1R homology model. The PPHT moiety (orange) binds D1R within the DA binding site (backbone residue in blue) of the receptor. Azobenzene (maroon) and the PEG linker (green) bind within the pore that leads to the DA binding site, and the PEG linker extends out of the receptor. (B) To optimize the length of the PEG linker in P-D1ago, it was compared to a MP that was developed previously for a metabotropic glutamate receptor (mGluR), benzylguanine-azobenzene-glutamate (BGAG). BGAG requires a long PEG linker (28 but not zero or 12 repeats) to reach from the SNAP-tag adjacent to the cell surface (the membrane anchor M) to the receptor binding site above the plasma membrane⁷⁷. However, if extended further, BGAG is less efficacious because it explores a wider three-dimensional space and is less concentrated. Therefore, MPs must not be too short or too long. Unlike mGluRs, DARs bind DA in the upper third of their transmembrane bundle. To provide structural insight into the length dependence of photoswitching, the minimal distance between the benzylguanine binding site in the M and the ligand binding site in either mGluR5 (left; pdb: 6n51) or a D1R homology model (right) was estimated. A model of the M was made by placing the C-terminus of SNAP-tag (pdb: 3L00) flush with the N-terminus of a single-pass transmembrane segment (pdb: 2K1A). The M was then positioned adjacent to mGluR5 or D1R. The benzylguanine binding site in the M (blue circle) was oriented facing towards the receptor ligand binding site. L-quisqualate (green) is shown bound to the ligand binding site of mGluR5. DA (red) is docked in the D1R homology model. (C) The lengths of the BGAG and P-D1ago were estimated. Their chemical structures are shown bound to SNAP-tag. Each photoswitch is composed of (i) benzylguanine that has reacted with and is bound covalently to a cysteine residue (Cys145) in SNAP-tag, (ii) a PEG linker with a hydrodynamic diameter (D_{H}) that depends on the number of ethylene glycol repeats (n), and (iii) azobenzene conjugated to a receptor ligand (glutamate in BGAG and PPHT in P-D1ago). (D) The hydrodynamic diameter (D_{H}) of PEG is shown as a function of increasing ethylene glycol repeats. D_{H} values were taken from multiple studies and averaged. In each study, D_{H} was measured at room temperature in water. D_{H} is not sensitive to changes in temperature, indicating that these values are valid for experiments performed at room temperature or in vivo studies. (E) The estimated length of BGAG and P-D1ago are shown as a function of increasing ethylene glycol repeats using published PEG D_{H}values (FIG. 7D). These results indicate that P-D1ago with 12-PEG repeats (~50 Å) is sufficiently long enough to span from the SNAP-tag in the M to the DA binding site in D1R (38 Å; FIG. 7B). However, even a BGAG with a long PEG linker (28 repeats; ~55 Å) would not effectively reach from the M to the mGluR ligand binding site (65 Å; FIG. 7B).

FIGS. 8A-8D. Photophysical properties of P-D1_{ago}. (A) Absorbance spectra of P-D1ago under blue light (460 nm; blue) and UV light (370 nm; grey). (B) P-D1ago repeatedly switches from its trans to cis isomer with UV light and back to trans with blue light. P-D1ago is stable in the cis state in the dark after illumination with UV light. (C) P-D1ago blocks the binding of the membrane impermeant dye SNAP-Surface Alexa Fluor 647 (BG-Alexa647) to the membrane-anchor (M) almost completely and is as effective as the impermeant benzylguanine analog, surface block. These results indicate that P-D1ago binds effectively to the M at the cell surface. Cells were incubated for 1 hour with vehicle (left upper panels), 1 µM surface block (left middle panels), or 1 µM P-D1ago (left lower panels), and were then labeled for 30 minutes with 1.5 µM BG-Alexa647. Cytosolic tdTomato was used as a transfection marker, grey bar = 10 µM. A summary of labeling according to flow cytometry is shown on the right. unpaired two-sided t-test, p = 0.60. n = 3-6 replicates per condition. (D) M labeling as a function of the concentration of P-D1_{ago}. The M was incubated for 1 hour with increasing concentrations of P-D1ago, and then labeled for 30 minutes with 1.5 µM BG-Alexa647. Labeling was measured by flow cytometry. (E) Time course of M labeling with P-D1_{ago}. The M was incubated for different times with 1 µM P-D1ago, and then labeled for 30 minutes with 1.5 µM BG-Alexa647. Labeling was measured by flow cytometry. n = 3-4 replicates per condition. (F) P-D1ago does not effectively prevent the binding of the membrane permeant dye SNAP-Cell TMR-Star (BG-TMRstar) to intracellular SNAP-tag that is expressed in the cytosol. This contrasts with the commercially available permeant benzylguanine analog, cell block. These results indicate that P-D1ago does not bind effectively to intracellular M. Cells were incubated for 1 hour with vehicle (left upper panels), 1 µM cell block (left middle panels), or 1 µM P-D1ago (left lower panels), and were then labeled for 20 minutes with 1.5 µM BG-TMRstar. Cytosolic mTurquoise was used as a transfection marker, grey bar = 10 µM. A summary of labeling according to flow cytometry is shown on the right. unpaired two-sided t-test, ***p < 0.001. n = 3 replicates per condition.

FIGS. 9A-9H. Functional properties of untethered P-D1_{ago}. (A) Schematic representation of a D1R-mediated G protein-coupled inwardly rectifying potassium (GIRK) channel assay in HEK293T cells. Agonist-induced receptor activation results in the recruitment of a heterotrimeric G protein containing the chimeric Gα subunit (Gαᵢₛ) followed by the release of Gβγ, which activates GIRK channels and enhances inward-current. (B) Dopamine (DA) dose-dependently activates D1R in the GIRK assay. The potency of DA (pEC50 = 6.3 ± 0.1) is comparable to that observed for endogenous DARs in intact tissue⁸⁶. n = 6-9 cells per concentration. (C) Representative traces of the effect of untethered P-D1ago on D1R in the absence of the membrane-anchor (M) according to the GIRK assay. Neither 1 µM or 10 µM P-D1_{ago}, concentrations that fully label the M (FIG. 8D), have any effect on the receptor in either the trans (blue light; blue bars) or cis (UV light; grey bars) isomeric state. (D) Summary of the functional effect of untethered P-D1ago on D1R in the GIRK assay. n = 4-5 cells per condition. (E) Schematic representation of a highly sensitive D1R-mediated cAMP accumulation assay in HEK293T cells. Agonist-induced D1R activation results in G_{s/olf}recruitment followed by the binding of its Gα subunit to adenylate cyclase (AC), which enhances the conversion of ATP to cAMP. cAMP binds the bioluminescence resonance energy transfer 1 (BRET′)-based cAMP sensor CAMYEL⁸⁷, resulting in a conformational change in its Epac domain that increases the distance and decreases BRET¹ between the donor Renilla luciferase (Rluc) and the acceptor YFP. (F) Representative dose-response curves of DA, 4-amino-PPHT (a parent molecule of P-D1_{ago}), or untethered P-D1ago induced activation of D1R according to the cAMP accumulation assay. n = 3 replicates per concentration. (G) Summary of the Eₘₐₓ of DA-, 4-amino-PPHT-, or untethered P-D1ago in the cAMP accumulation assay. one-way ANOVA, F = 0.1, Tukey. n = 3 experiments, each performed in triplicate per concentration. (H) Summary of the pEC50 of DA-, 4-amino-PPHT-, or untethered P-D1ago in the cAMP accumulation assay. one-way ANOVA, F = 43.9, Tukey, ***p < 0.001. n = 3 experiments, each performed in triplicate per concentration. Optimization and characterization of the D1R MP

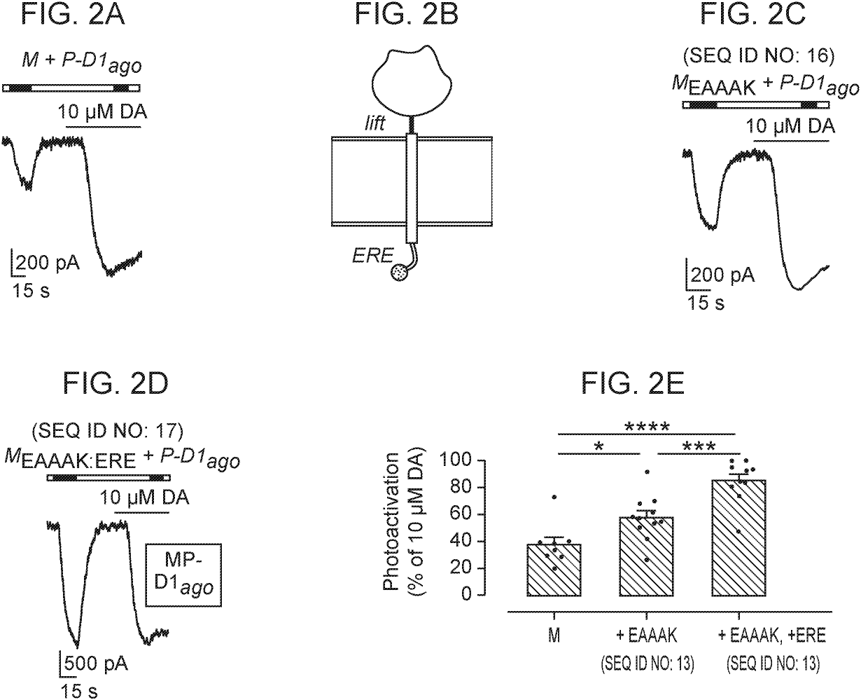

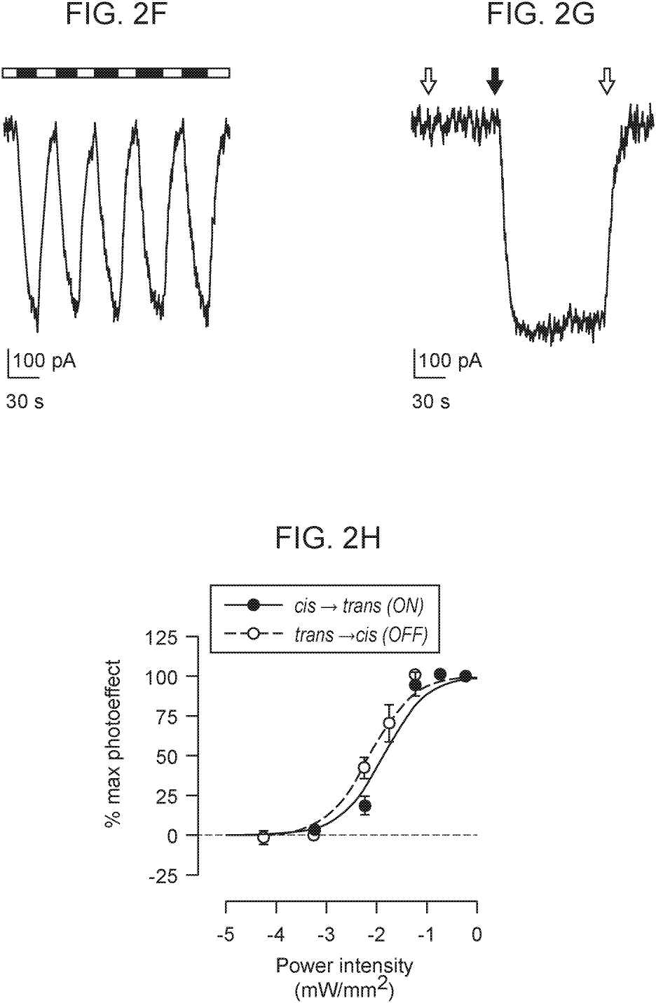

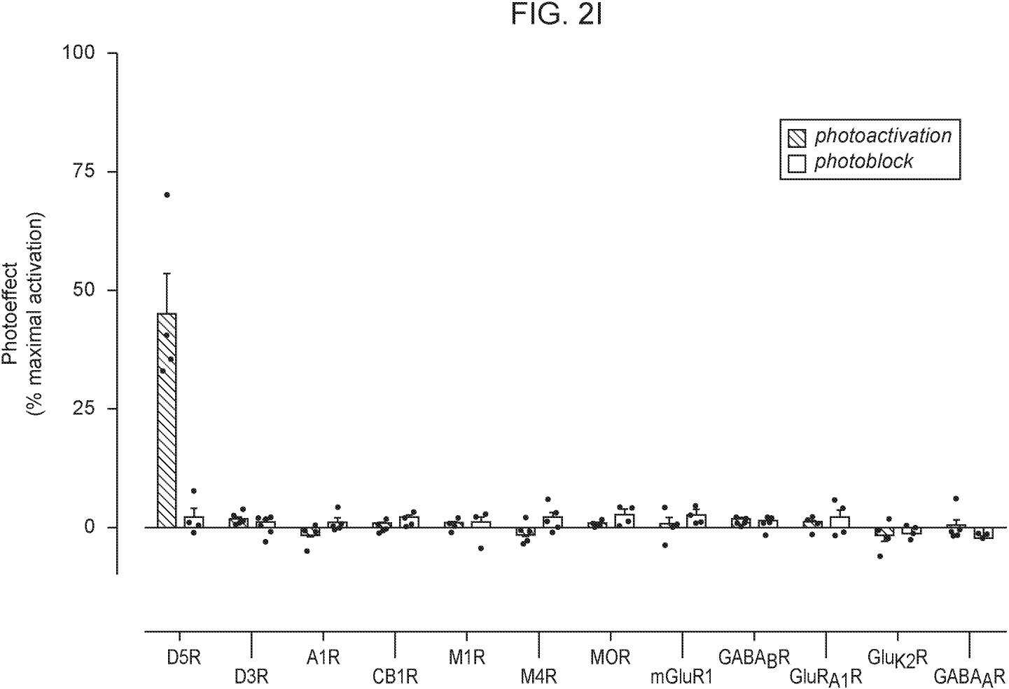

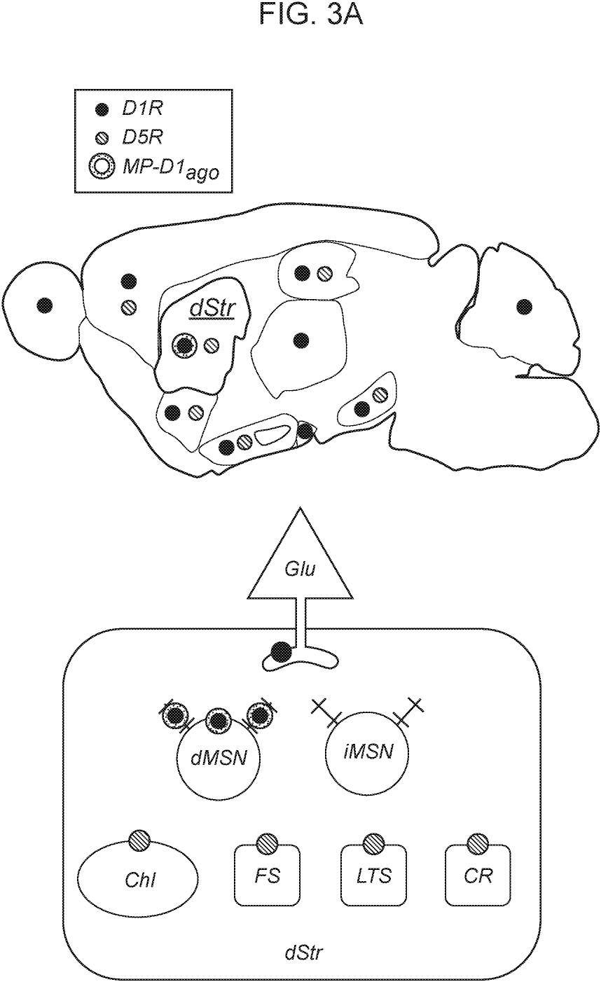

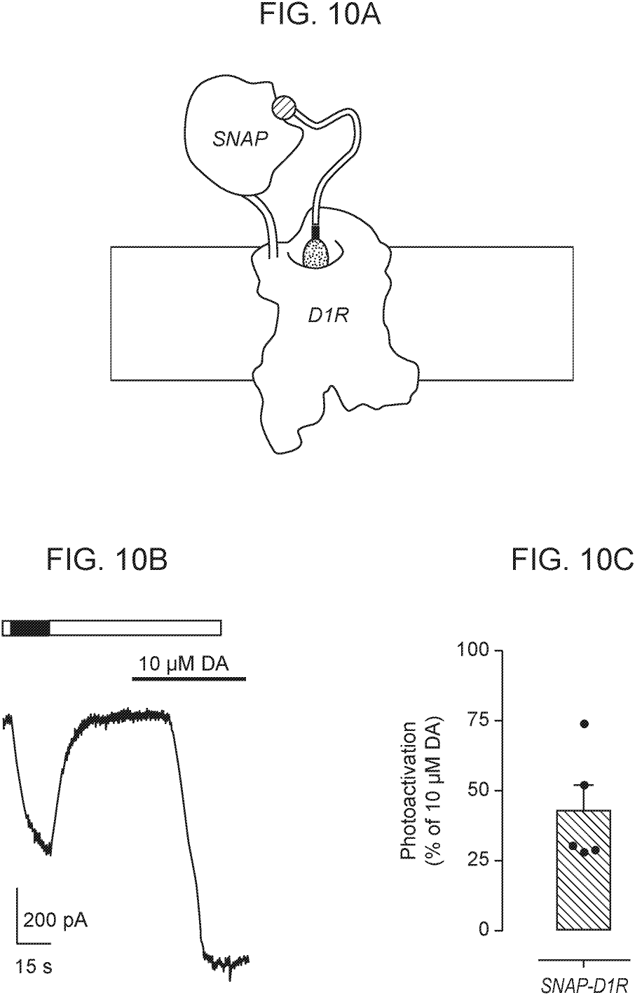

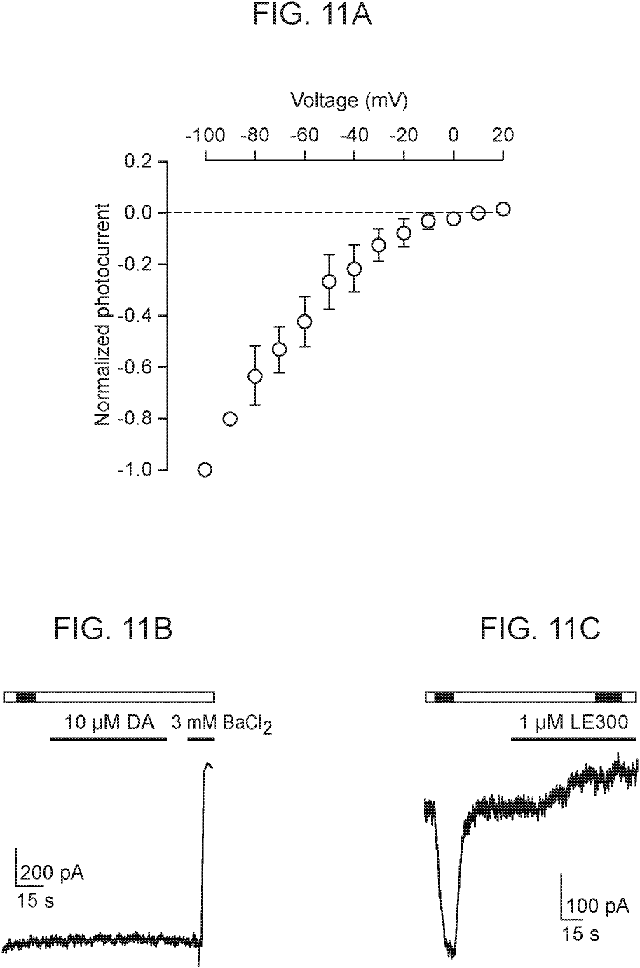

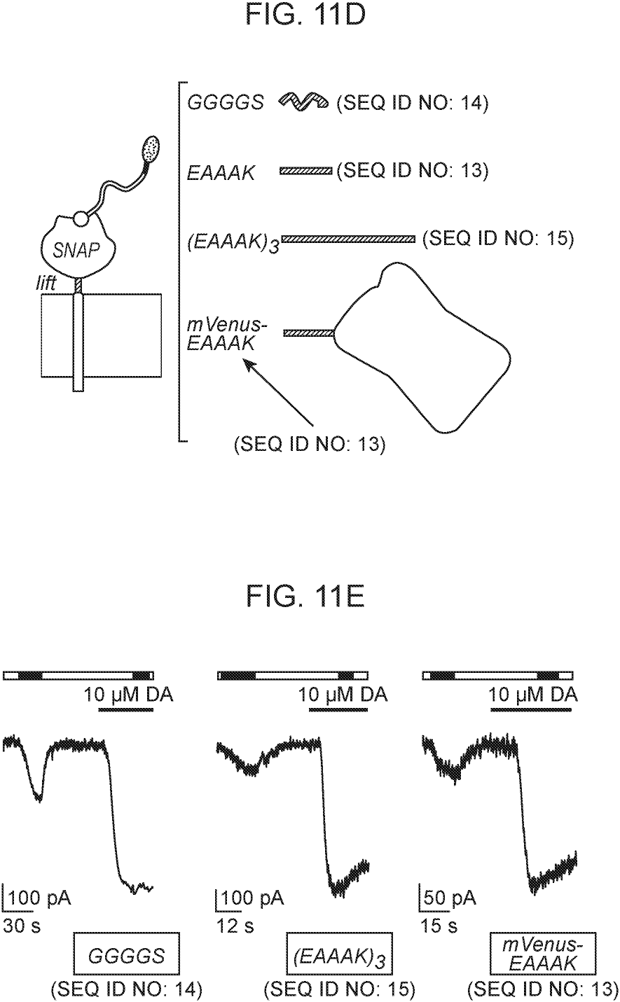

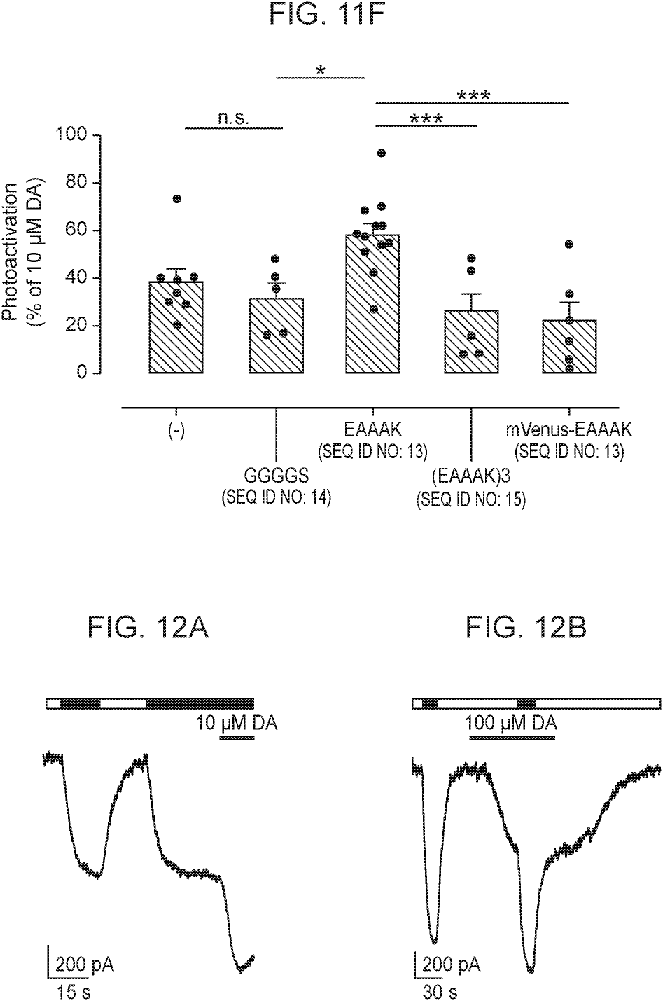

To test the ability of P-D1ago to photo-activate D1R, it was attached directly to a version of D1R that has SNAP fused to its extracellular N-terminus, which is adjacent to the ligand binding site of the receptor. SNAP-D1R was co-expressed in HEK293T cells with the G protein-coupled inwardly rectifying potassium (GIRK) channel as an effector. Following application and washout of P-D1ago, blue light (trans) activated and UV light (cis) deactivated GIRK current (FIG. 10), demonstrating P-D1ago as an effective light-switched tethered ligand. P-D1ago was attached to the M, which was co-expressed in HEK293T cells with the native D1R and the GIRK channel. In this inter-protein configuration, P-D1ago once again photoactivated ion current by blue light and was deactivated by UV light (FIGS. 2A and 2E), an effect that required expression of GIRK channels (FIG. 11A) and D1R (FIGS. 11B-11C), which indicated that inter-protein photo-activation of native D1R works. To optimize D1R photoactivation, the geometric positioning of P-D1ago was adjusted using variants of M with “lift” peptides between the SNAP-tag and transmembrane segment. Photoactivation increased with an M variant containing a rigid, α-helical EAAAK (SEQ ID NO: 13) peptide (M_{EAAAK} (SEQ ID NO: 16); FIG. 2B, C, and E) and decreased as the lift peptide was further extended (FIGS. 11D-11F), presumably because P-D1ago “rises” too far from the cell surface.

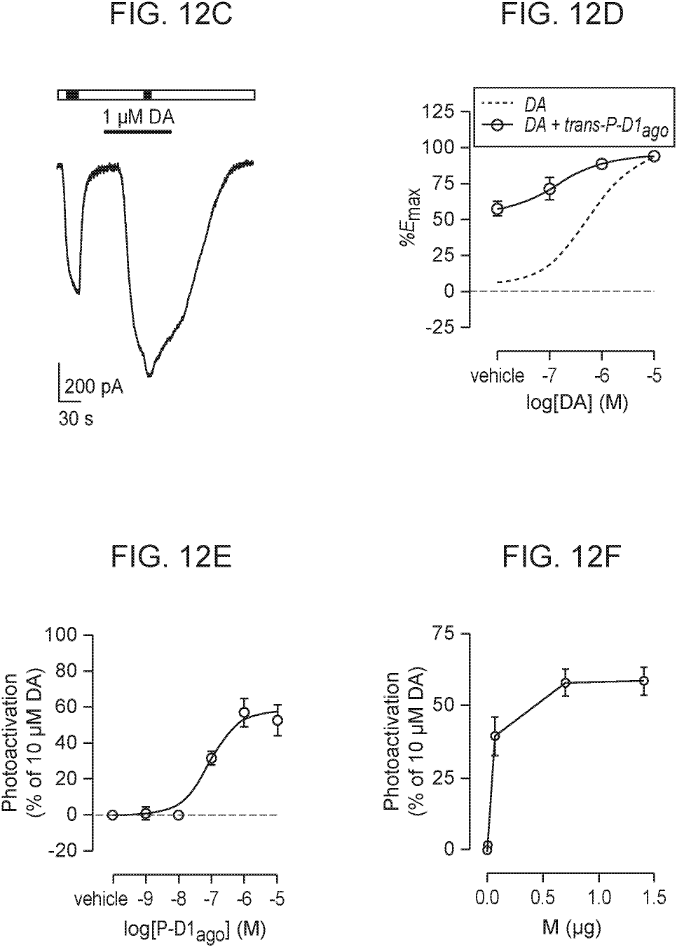

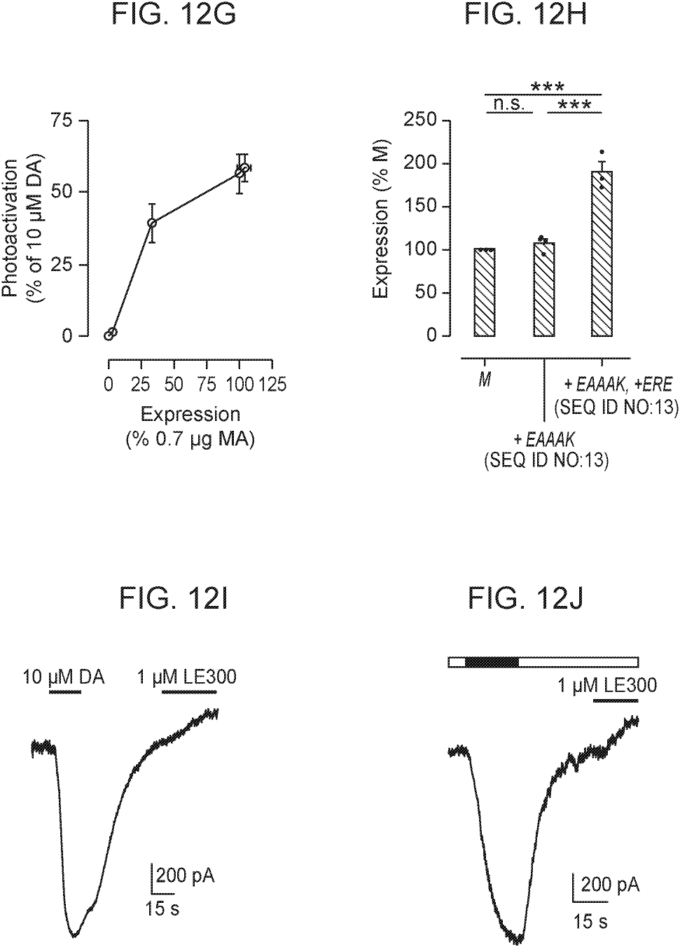

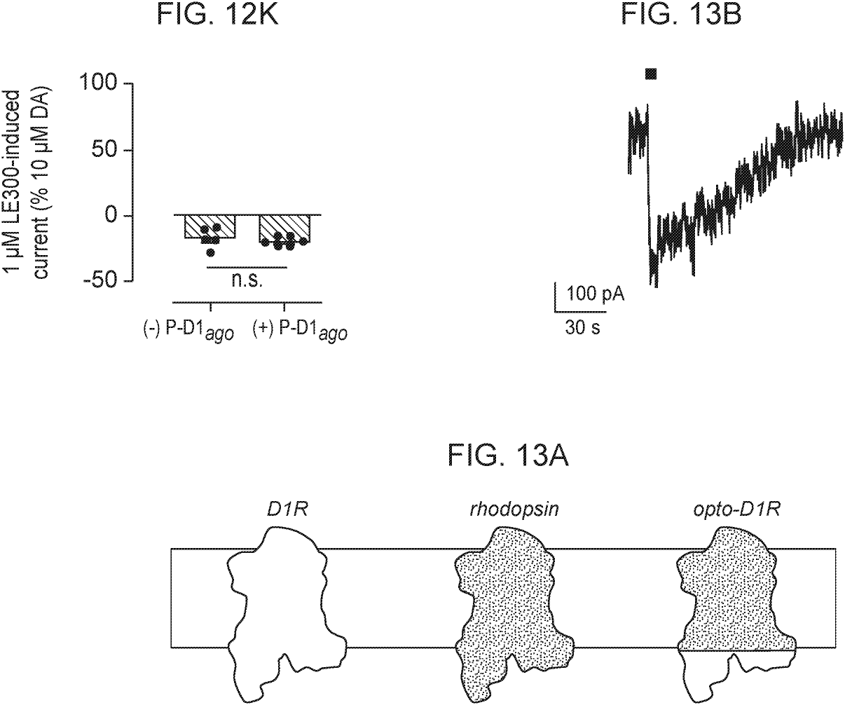

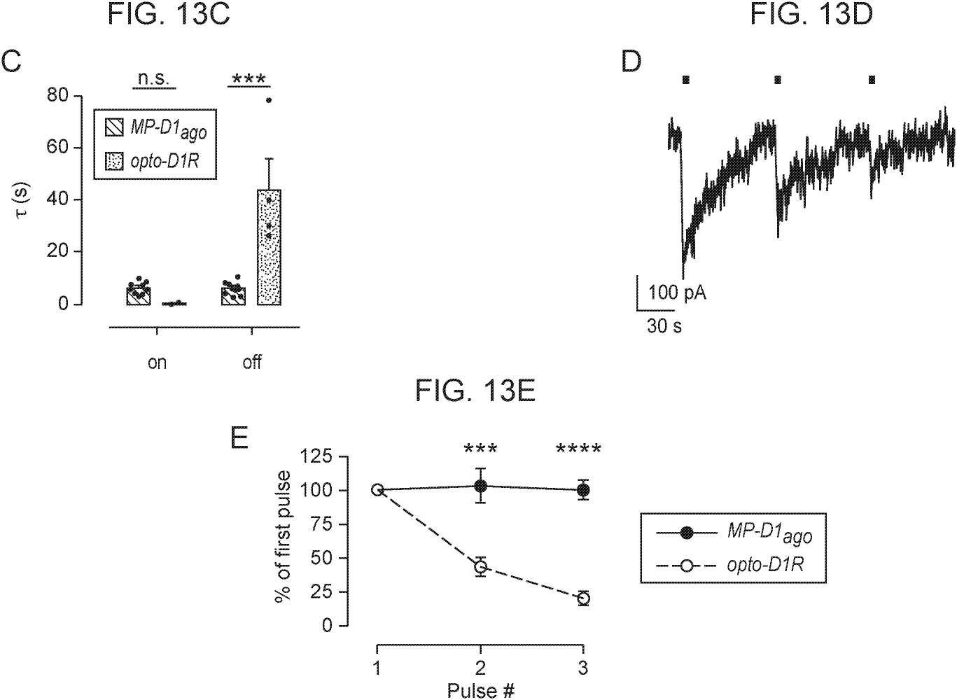

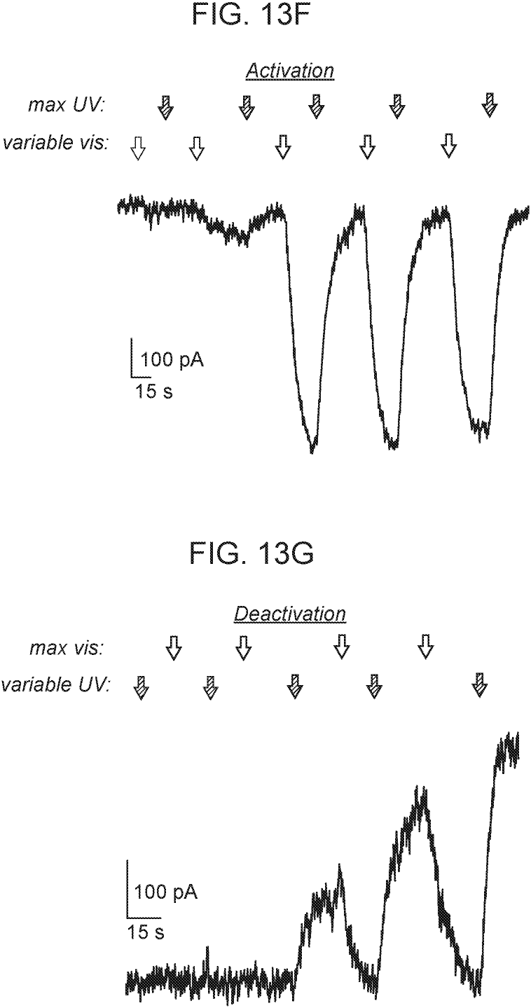

Pharmacological analyses suggested that when P-D1ago is tethered to M_{EAAAK} (SEQ ID NO: 16), it partially activates D1R because it is present at a submaximal concentration at the cell surface (FIGS. 12A-12G). To increase surface P-D1ago, an endoplasmic reticulum export ERE motif^{26,32} was added to the C-terminus of M_{EAAAK} (SEQ ID NO:16) (generating M_{EAAAK}:_{ERE}; SEQ ID NO: 17), which approximately doubled surface SNAP-tag expression (FIG. 12H). Remarkably, P-D1_{ago} tethered to M_{EAAAK:ERE} (SEQ ID NO: 17) (MP-D1_{ago}) that was stimulated by blue light activated D1R to near maximal possible levels (elicited by saturating, 10 µM, DA) (FIG. 2B, D, and E) without altering baseline activation in the “off-state, under UV light (FIGS. 12I-12K). GIRK current elicited by MP-D1_{ago} photo-activation and photo-deactivation of D1R rose and fell in seconds (FIG. 2F), with no loss in efficacy over multiple cycles (FIG. 2F), and in a bistable manner, i.e., the “on”-state was stable in the dark following a short flash of blue light and the “off”-state was stable after a short flash of UV light (FIG. 2G). This contrasts with slow off-kinetics and rundown of opto-D1R (FIGS. 13A-13E). MP-D1_{ago} was ~100-fold more sensitive to light than ChR2 (FIG. 2H; and FIGS. 13F and 13G)³⁴.

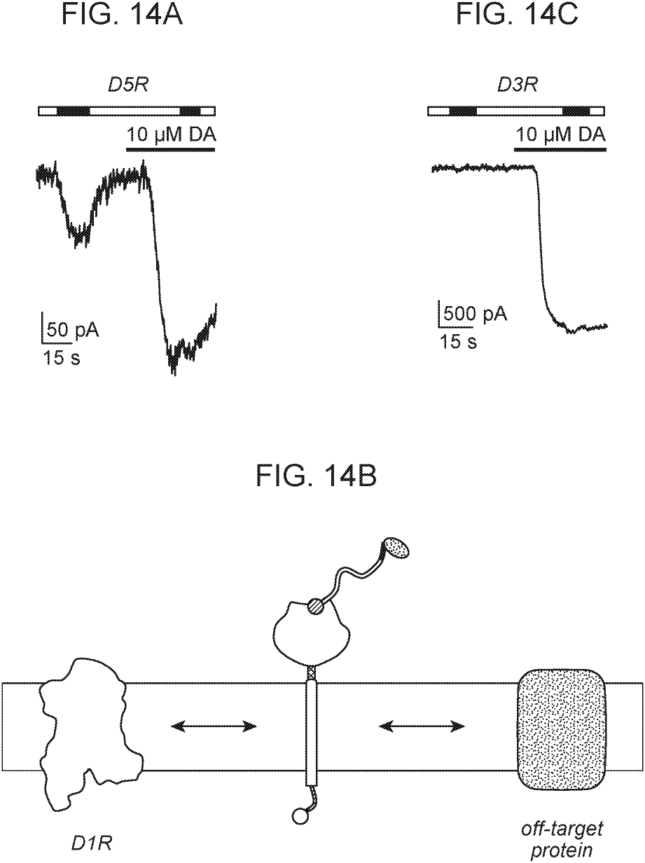

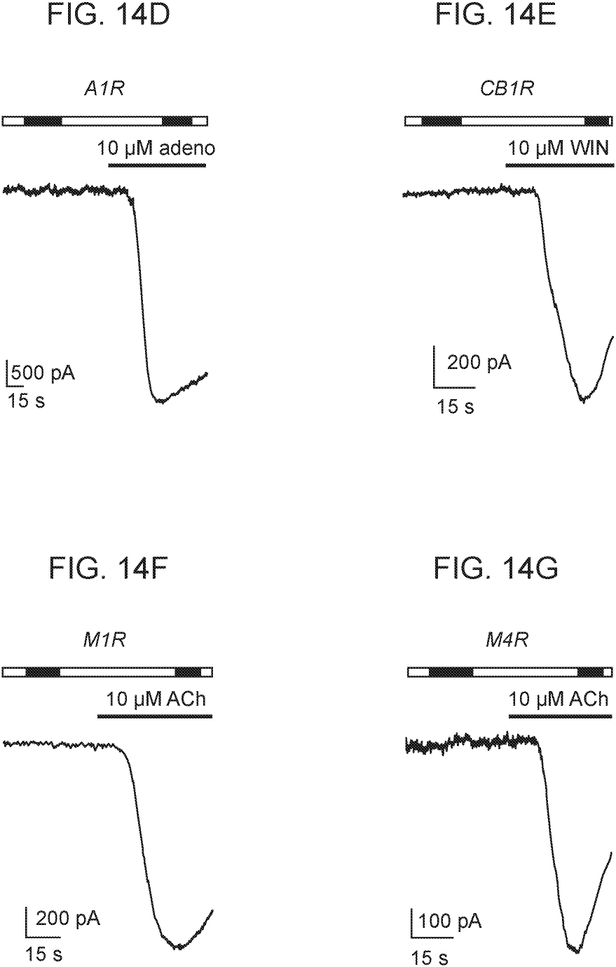

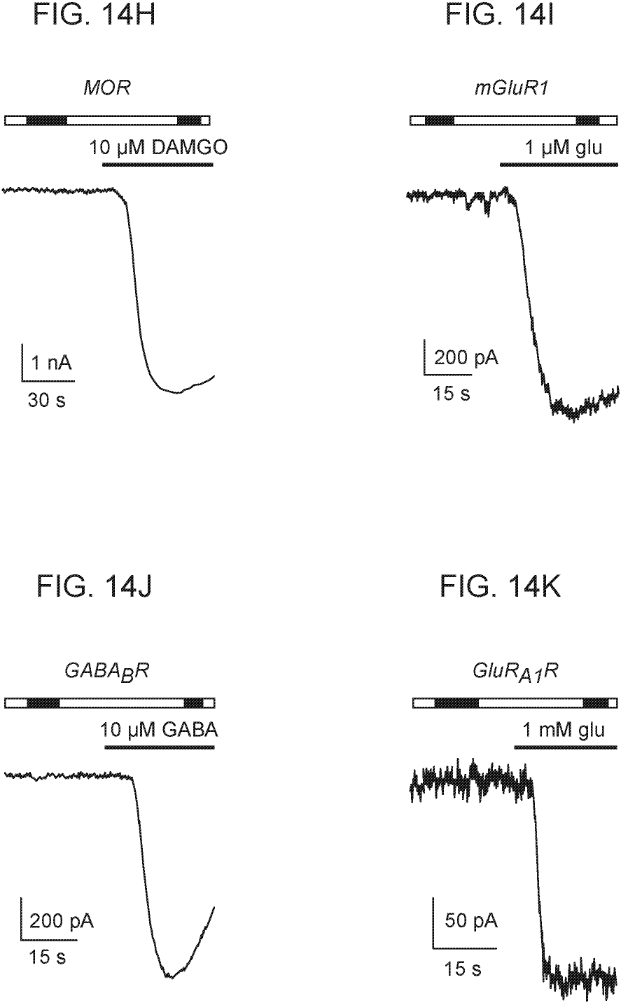

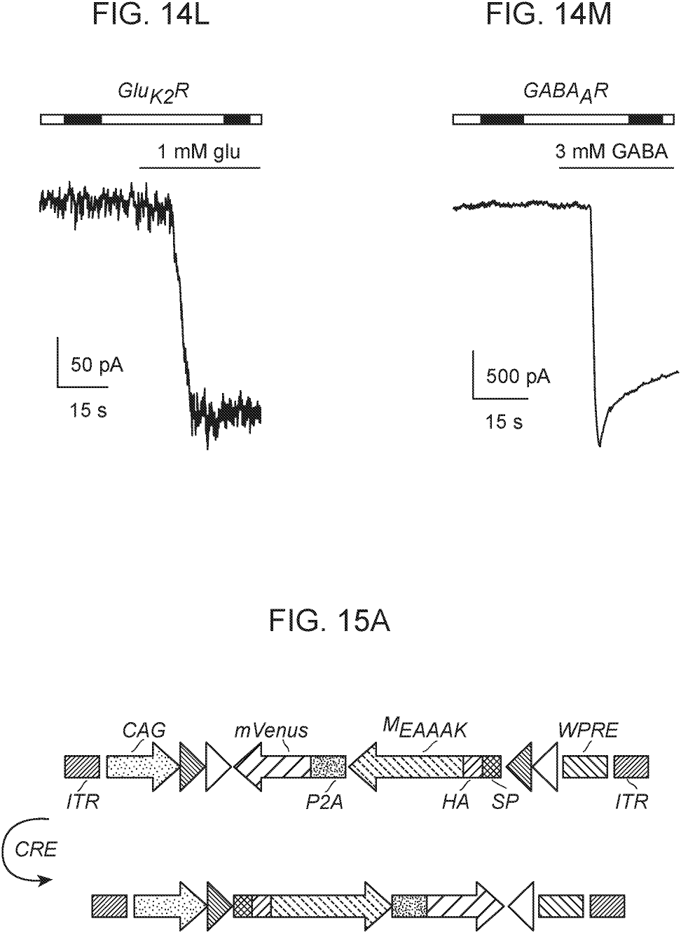

The specificity of MP-D1_{ago} was assessed by profiling its effects on a variety of neuronal receptors, each co-expressed in HEK293T cells with the M. D5R, the closest homolog of D1R, was tested. D5R was photoactivated by MP-D1_{ago} to about half the level of D1R (FIG. 2I; FIG. 14A). Other receptors that are co-expressed with D1R in the brain, including GPCRs and ionotropic receptors for DA, adenosine, cannabinoids, acetylcholine, opioids, glutamate, and GABA, were evaluated. MP-D1_{ago} had no effect on any of these receptors (FIG. 2I; FIGS. 14B-14M), indicating that it is a DlR/D5R-selective photo-agonist.

FIGS. 2A-2I. MP-D1_{ago} photoactivates D1R. (A) Photoactivation of D1R by P-D1ago tethered to the M, according to a receptor mediated-GIRK activation assay in HEK293T cells. The receptor is partially photoactivated with blue light (460 nm) and is deactivated with UV light (370 nm) relative to a saturating concentration of DA. (B-D) The incorporation of the rigid lift peptide EAAAK (SEQ ID NO: 13; dark green) and an endoplasmic reticulum export motif (ERE; purple) in the M enhances photoactivation of D1R. P-D1ago attached to M_{EAAAK:ERE} (SEQ ID NO: 17) is MP-D1_{ago}. (E) Summary of photoactivation of D1R with variants of the MP with different Ms. one-way ANOVA, F = 22.5, Tukey, *p < 0.05, ***p < 0.001, ***p < 0.0001. n = 8-12 cells per condition. (F) Photoactivation of D1R with MP-D1_{ago} is rapid, reversible, and repeatable. (G) Photoactivation of D1R with MP-D1_{ago} is bistable. (H) Photoactivation and deactivation of D1R with MP-D1_{ago} as a function of light intensity, n = 5 cells per condition. (I) Summary of photoactivation or photoblock by MP-D1_{ago} of selected receptors coexpressed with D1R in vivo. MP-D1_{ago} photoactivation of D5R is significantly greater than that of all other receptors tested. one-way ANOVA, F = 27.5, Tukey, p < 0.0001. n = 4-6 cells per condition

FIGS. 10A-10C. Functional characterization of the effect of P-D1ago tethered to SNAP-D1R. (A) Schematic representation of P-D1ago tethered to a SNAP-tag fused directly to the extracellular N-terminus of D1R. (B) Representative trace of SNAP-D1R photoactivation by P-D1ago according to the GIRK assay. P-D1ago photoactivates D1R in response to blue light and is deactivated with UV light. (C) Summary of SNAP-D1R photoactivation by P-D1ago relative to a saturating concentration of DA (10 µM). n = 5 cells.

FIGS. 11A-11F. Functional characterization of the effect of P-D1ago tethered to M or its variants on D1R. (A) Inward-current in response to D1R photoactivation with P-D1ago tethered to the SNAP-tag containing membrane anchor (M) as a function of voltage (photo-IV curve) in HEK293T cells. Switching from UV light to blue light has a negligible effect on inward-current at positive voltages but decreases exponentially as voltage becomes more negative, consistent with the current-voltage relationship associated with activation and opening of GIRK channels⁸⁸. n = 3 cells per voltage. (B) Switching from UV light to blue light has no effect on the basal current of 1 µM P-D1ago labeled HEK293T cells expressing GIRK channels but not D1R (1.3 ± 1.0% of 3 mM BaCh, a GIRK channel blocker, paired two-sided t-test, p < 0.01. n = 5 cells). (C) Photoactivation of D1R by P-D1ago tethered to the M is abolished in the presence of the D1R antagonist LE300 (3.3 ± 3.3% of photocurrent in the absence of LE300. paired two-sided t-test, p < 0.01. n = 5-6 cells per condition). The decrease in inward-current in response to LE300 results from its actions as an inverse agonist that reduces constitutive receptor activity⁸⁹. (D) Schematic representation of M analogs with lift peptides of different lengths and physical characteristics. (E) Representative traces of D1R photoactivation with P-D1ago tethered to M variants with different lift peptides. (F) Summary of photoactivation of D1R by P-D1ago with M variants with different lift peptides. one-way ANOVA, F = 17.1, Tukey, *p < 0.05, ***p < 0.001. n = 5-12 cells per condition

FIGS. 12A-12K. Increasing the surface level of membrane-anchored P-D1ago enhances D1R photoactivation without increasing basal receptor activation. (A) P-D1ago tethered to M_{EAAAK} (SEQ ID NO:16) only partially activates D1R under blue light (FIGS. 2a,e), which could be because it is either a partial agonist or a full agonist that partially occupies the receptor. To test this, photoactivation in the presence of DA was evaluated. D1R can be further activated by a saturating concentration of DA (10 µM) when preactivated by P-D1ago tethered to M_{EAAAK} (SEQ ID NO: 16) according to the GIRK assay. Thus, unlike a partial agonist, P-D1ago tethered to M_{EAAAK} (SEQ ID NO: 16) did not diminish the ability of DA to fully activate D1R. Moreover, consistent with the actions of a full agonist, P-D1ago tethered to M_{EAAAK} (SEQ ID NO: 16) further activated D1R in the presence of submaximal (100 nM; B) and near maximal (1 µM; C) concentrations of DA. (D) Summary of D1R photoactivation with P-D1ago tethered to M_{EAAAK} (SEQ ID NO: 16) in the presence of sub-saturating concentrations of DA. The dotted line represents the dose-response curve of activation of D1R by DA in the absence of P-D1ago tethered to M_{EAAAK}. (SEQ ID NO: 16) n = 3-10 for each DA concentration. (E) The level of P-D1_{ago} tethered to M_{EAAAK} (SEQ ID NO: 16) is not limited by submaximal SNAP-tag labeling with P-D1_{ago}. D1R photoactivation by P-D1ago tethered to M_{EAAAK} (SEQ ID NO: 16) is shown as a function of the labeling concentration of P-D1_{ago}. M_{EAAAK} (SEQ ID NO: 16) was labeled for 1 hr at 37° C. with increasing concentrations of P-D1_{ago}. n = 3-10 for each concentration of P-D1_{ago}. (F) The level of P-D1_{ago} tethered to M_{EAAAK} (SEQ ID NO: 16) is not limited by insufficient M_{EAAAK} (SEQ ID NO:16) cDNA. D1R photoactivation by P-D1ago tethered to M_{EAAAK} (SEQ ID NO: 16) as a function of the amount of M_{EAAAK} (SEQ ID NO: 16) DNA used to transfect HEK293T cells. M_{EAAAK} (SEQ ID NO: 16) was labeled for 1 hr at 37° C. with 1 µM P-D1_{ago}. n = 3-10 for each amount of DNA. (G) D1R photoactivation by P-D1ago tethered to M_{EAAAK} (SEQ ID NO: 16) as a function of the surface levels M_{EAAAK} (SEQ ID NO: 16). Surface levels of M_{EAAAK} (SEQ ID NO: 16) were quantified by labeling with BG-Alexa647 and measuring fluorescence using flow cytometry. n = 3 for each measure of expression, n = 3-10 for each measure of photoactivation. (H) Surface levels of variants of M containing the lift peptide EAAAK (SEQ ID NO: 13) and the endoplasmic reticulum export tag ERE. Surface levels were quantified by labeling M expressing cells with BG-Alexa647 and measuring fluorescence using flow cytometry. one-way ANOVA, F = 40.2, Tukey, p = 0.001. n = 3. (I) Representative trace of the effect of the D1R inverse agonist LE300 on basal inward-current of D1R-expressing HEK293T cells in the GIRK assay. (J) Representative trace of the effect of the D1R inverse agonist LE300 on basal inward-current of D1R- and M_{EAAAK}:_{ERE}-expressing (SEQ ID NO: 17) HEK293T cells labeled with P-D1ago in the GIRK assay. (K) Summary of the effect of LE300 on basal inward current of D1R- and M_{EAAAK}:_{ERE}-expressing (SEQ ID NO: 17) cells labeled or not labeled with P-D1_{ago}. unpaired t-test, p = 0.46. n = 5-6 cells per condition.

FIGS. 13A-13G. Functional properties of MP-D1_{ago} compared to opto-D1R. (A) Schematic representation of opto-D1R, a chimeric protein consisting of the transmembrane domain of the inherently light sensitive GPCR rhodopsin in combination with the intracellular signaling components of D1R. When ectopically expressed in the target cell type, this engineered protein can be activated remotely with light and engage D1R-mediated signaling with cell type and spatiotemporal specificity. (B) Representative trace of the activation of opto-D1R with 473 nm light in the GIRK assay. (C) D1R deactivates within seconds with MP-D1_{ago}, but opto-D1R takes greater than one minute to turn off completely. one-way ANOVA, F = 25.4, Tukey, ***p < 0.001. n = 4-11 cells per condition. (D) Representative trace of repeated cycles of activation of opto-D1R with 473 nm light. (E) D1R photoactivation with MP-D1_{ago} is repeatable with no loss in efficacy after multiple cycles. However, peak activation of opto-D1R drops off dramatically (~5-fold reduction after two cycles). Moreover, whereas MP-D1_{ag0} can sustain D1R in an active state following a brief exposure of light opto-D1R requires continuous light exposure to remain active, one-way ANOVA, F = 28.0, Tukey, ***p < 0.001, ****p < 0.0001. n = 3-8 per cells per condition. Representative traces of MP-D1_{ago} photoactivation (F) and deactivation (G) with increasing light intensities. See FIG. 2H for summary. Arrows indicate a one second flash of light.

FIGS. 14A-14M. MP-D1_{ago} is a D1R/D5R selective-photoagonist. (A) MP-D1_{ago} partially photoactivates D5R, the most closely related receptor to D1R (~80% homology within the transmembrane domain). D5R was coexpressed with the chimeric G protein Gαᵢₛ₁₃, allowing the receptor to couple to GIRK channels (B) MPs like MP-D1_{ago} are restricted to the surface of selected neurons, preventing them from binding the receptor of interest and off-target proteins in other neurons. However, MPs laterally diffuse across the plasma membrane and so can in principle interact with off-target proteins that are co-expressed alongside the receptor of interest in the same cell. Thus, the selectivity of the MP must be conferred by the inherent specificity of the photoswichable ligand. MP-D1_{ago} has no effect on D3R (C), a DAR that is coexpressed with D1R in vivo but has low homology in the transmembrane domain (-39%). MP-D1_{ago} also has no effect on other GPCRs that are reportedly expressed in D1R-expressing neurons in vivo, including adenosine A1R (D), cannabinoid CB1R (E), acetylcholine receptors M1R (F) and M4R (G), opioid MOR (H), metabotropic glutamate receptor mGluR1 (I), and GABA GABA_{B}R (J). In all cases, GPCR-mediated GIRK channel activation was measured. D3R was coexpressed with Gα_{oA}, and M1R and mGluR1 were coexpressed with the chimeric G protein Gα_{iq5}, allowing the receptors to couple to GIRK channels in HEK293T cells. Furthermore, MP-D1_{ago} has no effect on selected ionotropic receptors that are widely expressed in the brain: the AMPA receptor GluR_{AI}R (K), the kainite receptor Glu_{K2}R (L), and the GABA receptor GABA_{A}R_{α1β2γ2} (M). A non-desensitizing mutant of GluR_{A1}R (L497Y) was used. To prevent the desensitization of Glu_{K2}R, 0.3 mg/mL concanavalin A was added to the extracellular solutions. abbreviations: dopamine = DA, adenosine = adeno, WIN = WIN55,212-2, acetylcholine = ACh, glu = glutamate.

### Activation of D1Rs in the Dorsal Striatum With MP-D1_{ago} promotes movement initiation

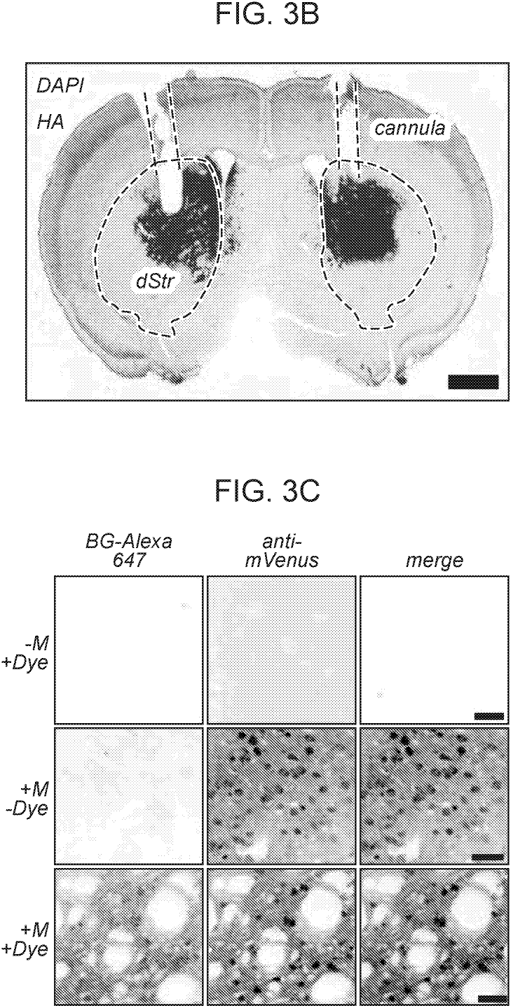

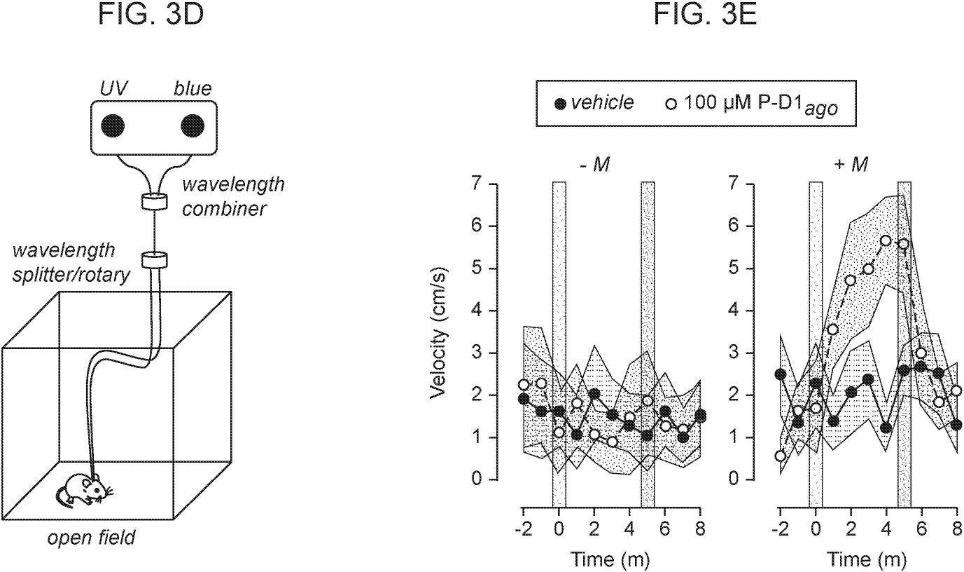

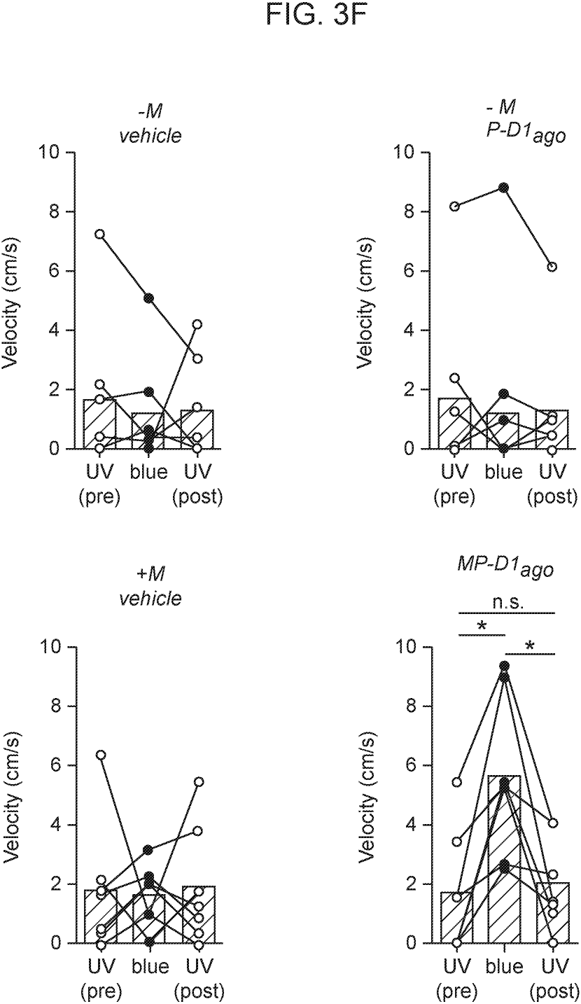

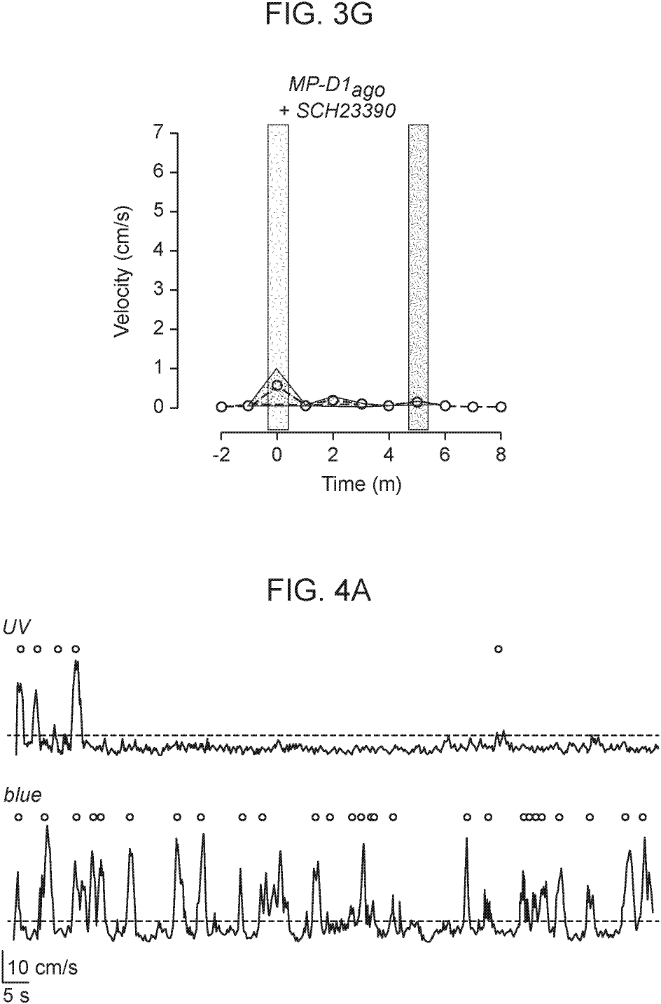

The dStr expresses D1R and D5R (FIG. 3A), which are notoriously difficult to distinguish with conventional pharmacology. However, dStr D1Rs and D5Rs are segregated into distinct classes of neurons: D1R is found in all dMSNs and the terminals of some glutamatergic afferents that innervate the dStr, whereas D5R is found in four types of interneurons (FIG. 3A). Thus, the targeted delivery of MP-D1_{ago} to dStr-dMSNs in combination with its rapid on and off kinetics would allow us to control of dStr D1Rs with specificity and determine its effect on movement initiation.

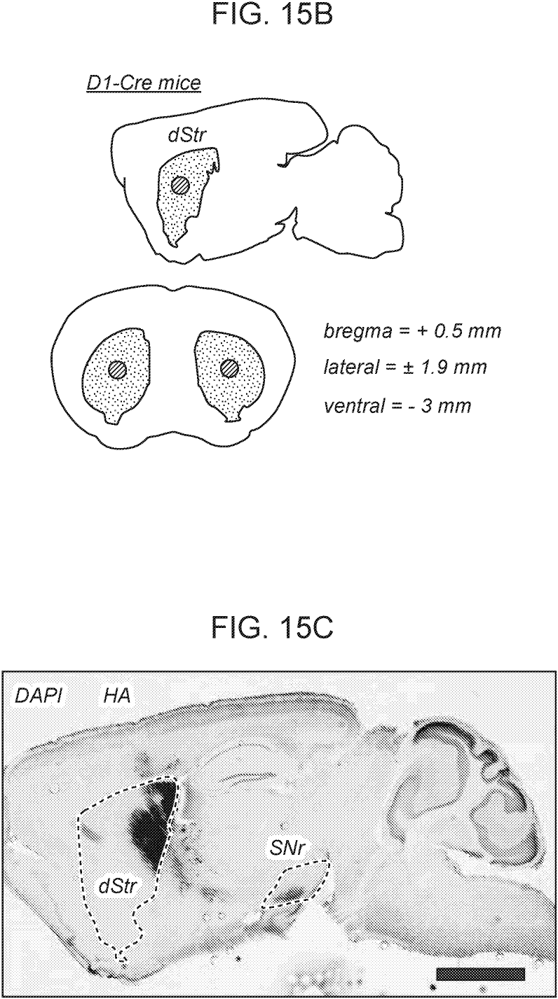

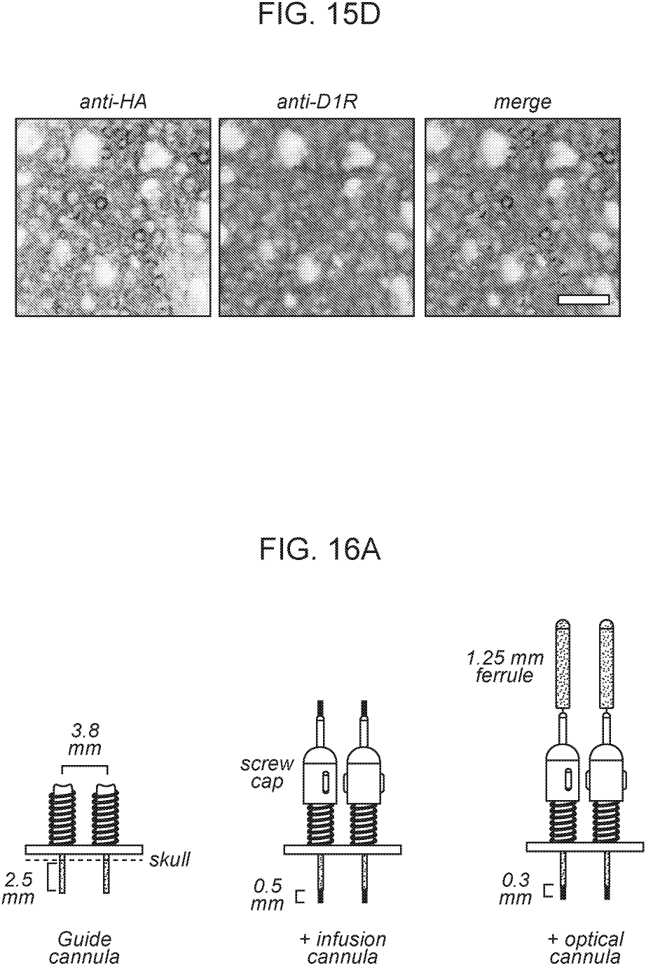

The gene encoding the M component of MP-D1_{ago}, M_{EAAAK:ERE} (SEQ ID NO: 17), was delivered to dStr-dMSNs in D1-Cre mice (FIG. 3A) using a Cre recombinase (Cre)-dependent adeno-associated virus (AAV) that was stereotaxically injected (FIGS. 15A and 15B). An AAV containing only mVenus was used as a control. The M expressed throughout the dStr (FIG. 3B; and FIG. 15C), on the cell surface of dStr-dMSNs (FIG. 3C), and alongside endogenous D1Rs in the soma and neurites (FIG. 15D) — prerequisites for MP-D1_{ago} to interact with its target receptor.

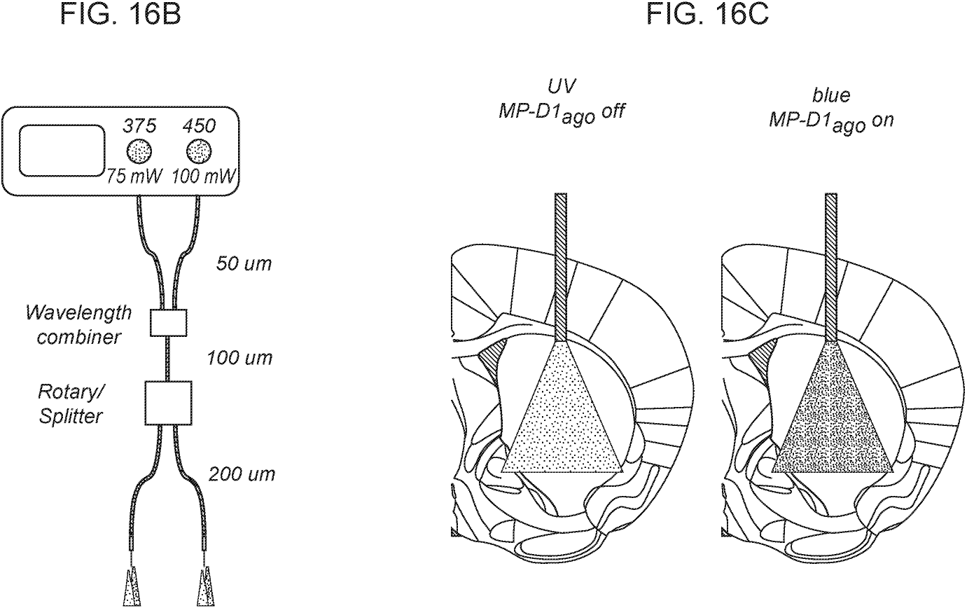

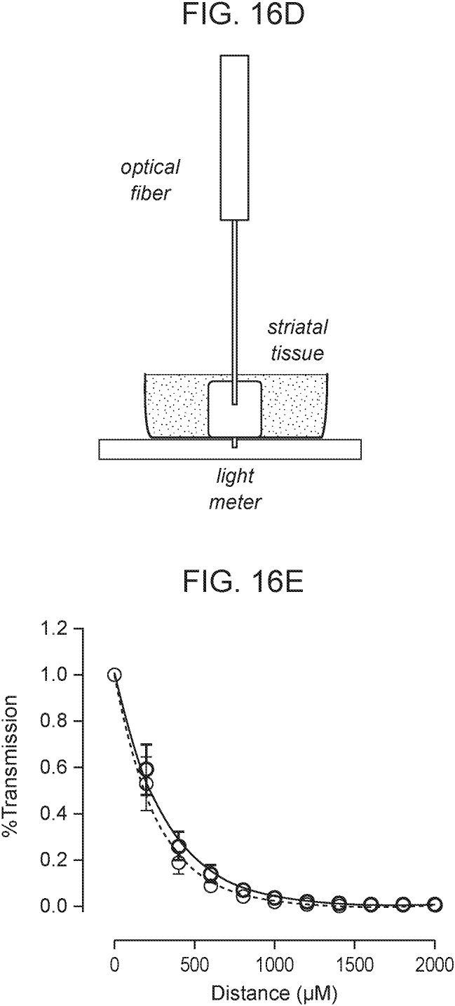

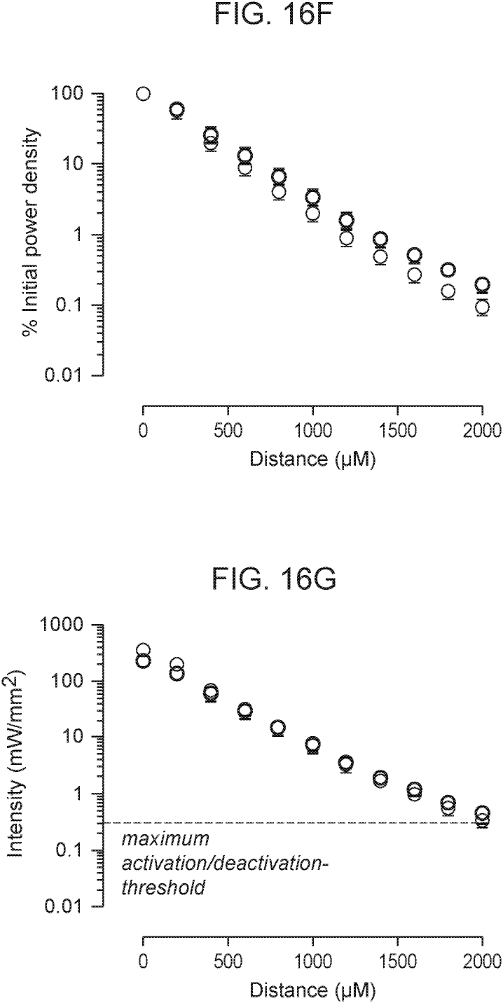

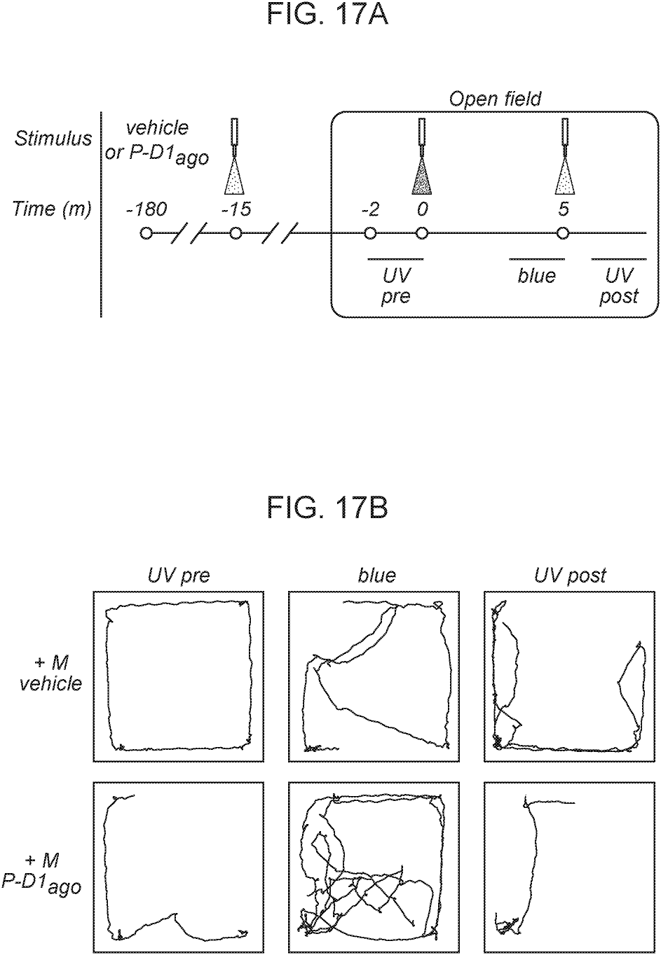

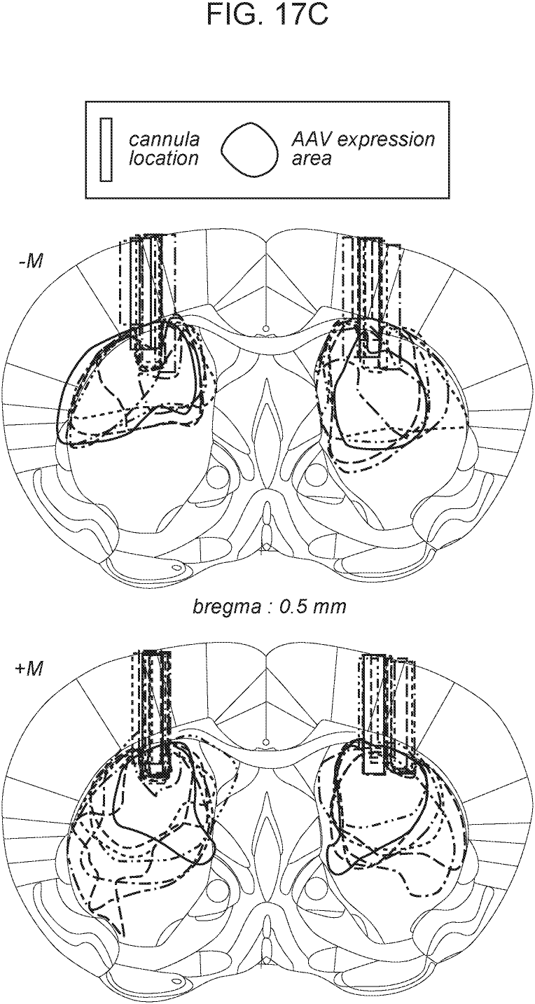

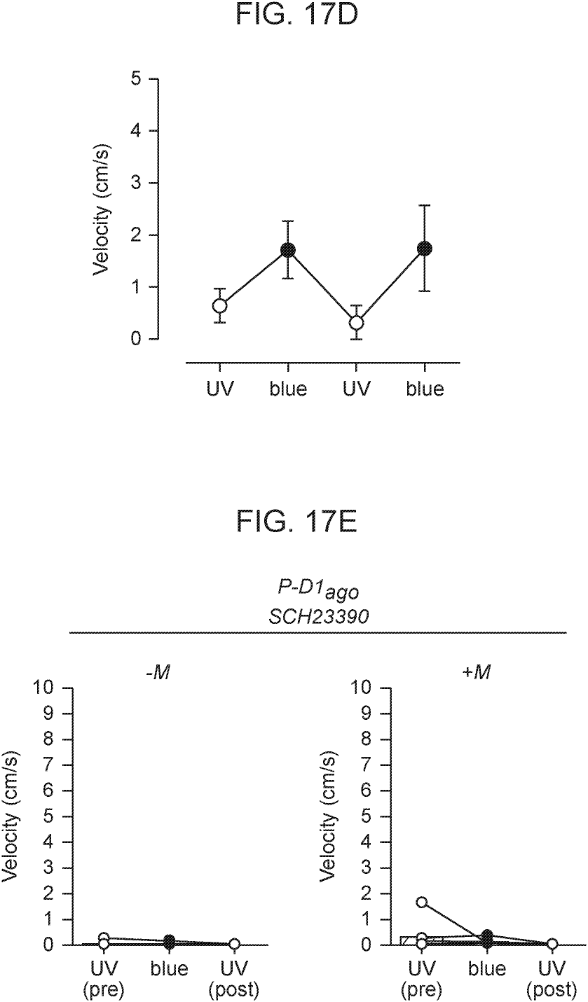

Mice expressing the M in dStr-dMSNs were infused with the UV-light induced inactive configuration of P-D1ago (cis). P-D1ago was incubated with the M for three hours to allow for conjugation. Locomotion was measured in an open field under conditions of optimized bilateral illumination (FIG. 3D; and FIG. 16). Locomotion of mice with MP-D1_{ago} increased following a flash of blue light (1 s) and returned to baseline following a flash of UV light (1 s; FIG. 17A), an effect that was robust (FIG. 3E; and FIGS. 17B and 17C) and repeatable (FIG. 17D). The behavioral photo-effect was not observed in the absence of either the M or P-D1ago (FIGS. 3E and 3F). MP-D1_{ago} was also tested in the presence of the D1R antagonist SCH23390, which suppresses locomotion on its own. The blue-light induced promotion of locomotion by MP-D1_{ago} was blocked by SCH23390 (FIG. 3G; and FIG. 17E), consistent with a requirement for D1R to confer the MP-D1_{ago} effect, though complicated by the suppression of baseline locomotion by SCH23390.

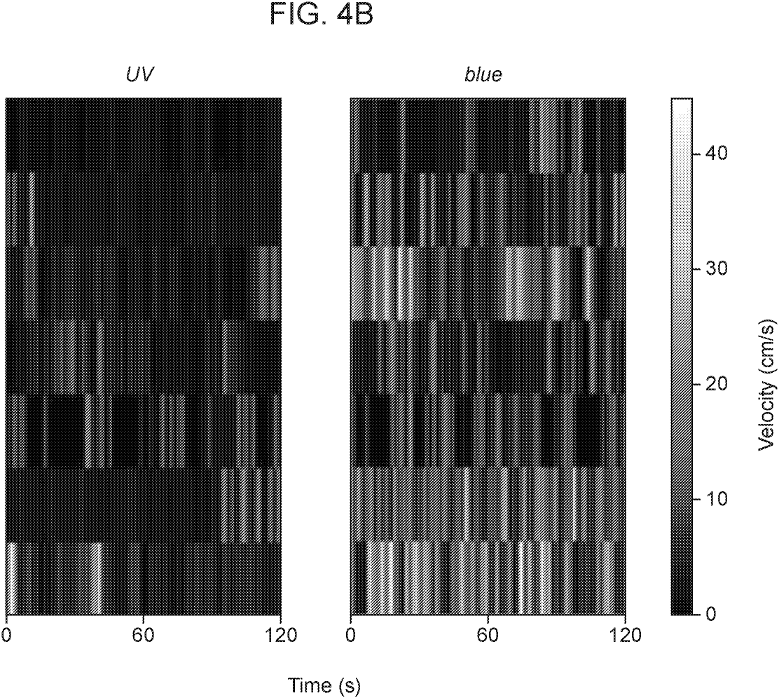

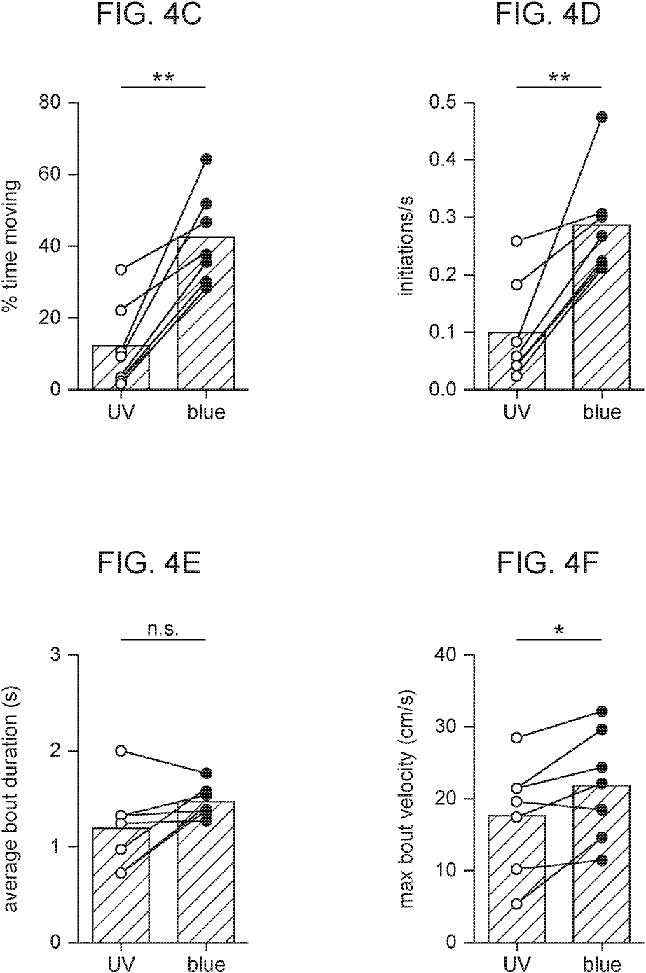

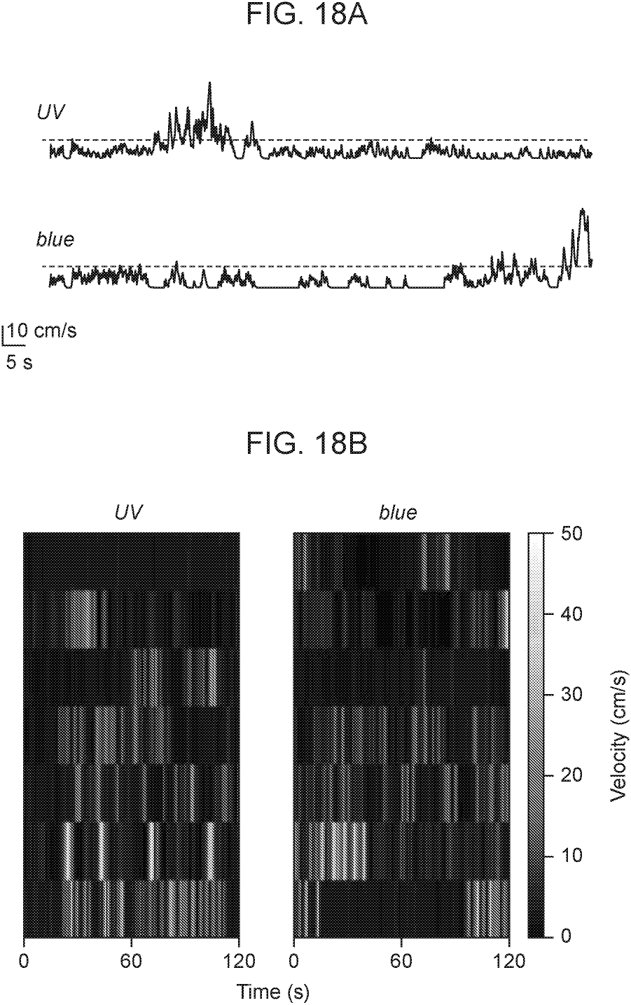

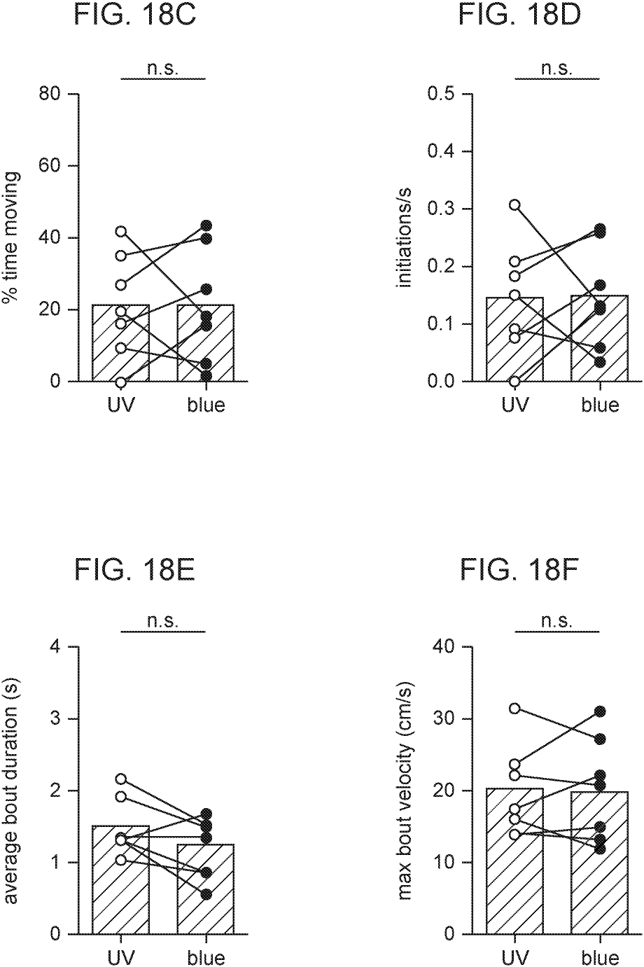

To better understand the basis of the locomotor response to dStr-dMSN D1R activation, the locomotion dynamics of mice were measured as MP-D1_{ago} was photoswitched from the inactive state (UV illumination) to the active state (blue illumination; FIG. 3E). The mice locomoted for a greater percentage of the time under blue light (FIGS. 4A-4C). The increase in locomotion was due to an increase in movement initiation (FIG. 4D) without a change in movement bout duration (FIG. 4E). There was also a modest increase in the maximum speed of motion in response to blue light (FIG. 4F). None of these differences were observed in mice lacking MP-D1_{ago} (FIG. 18), and there was no difference in the time spent in the center of the open field under UV light and blue light (FIG. 4G). Therefore, activation of D1R in dStr-dMSNs increases locomotion by increasing the probability of movement initiation.

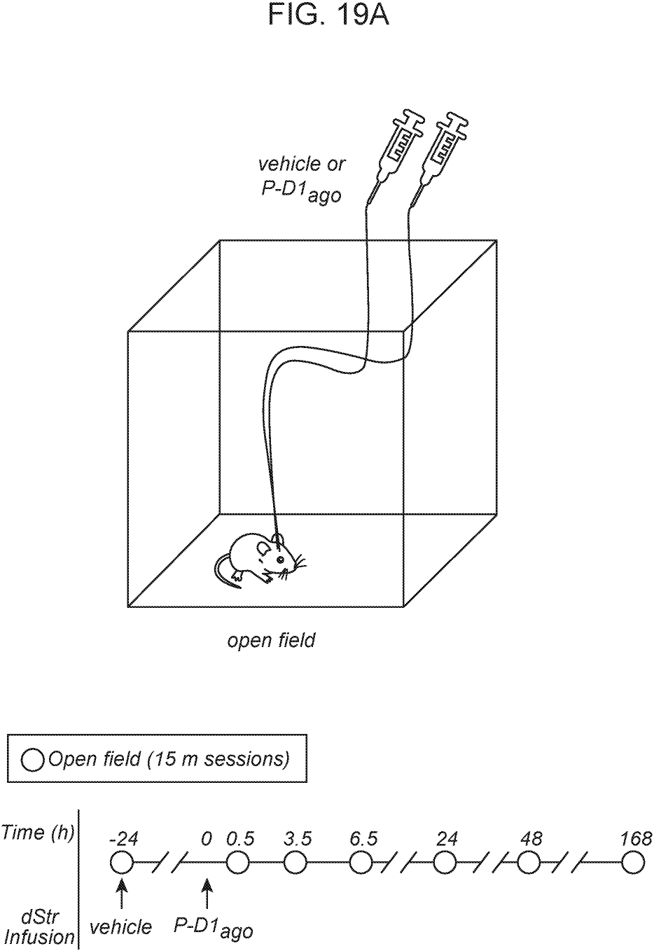

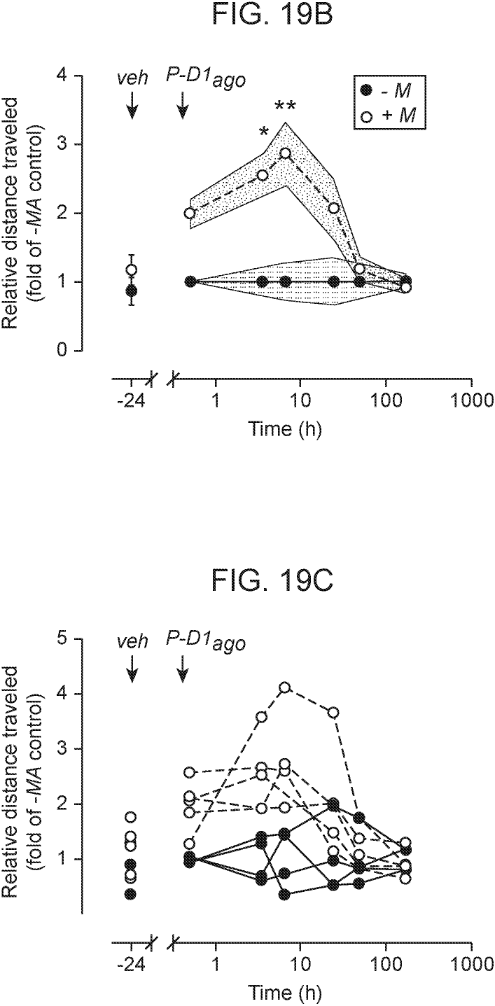

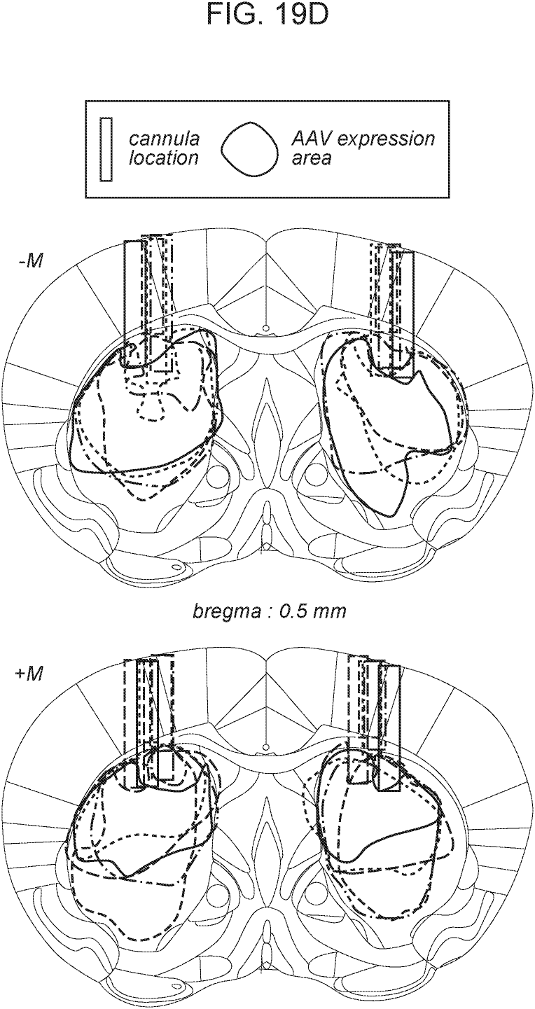

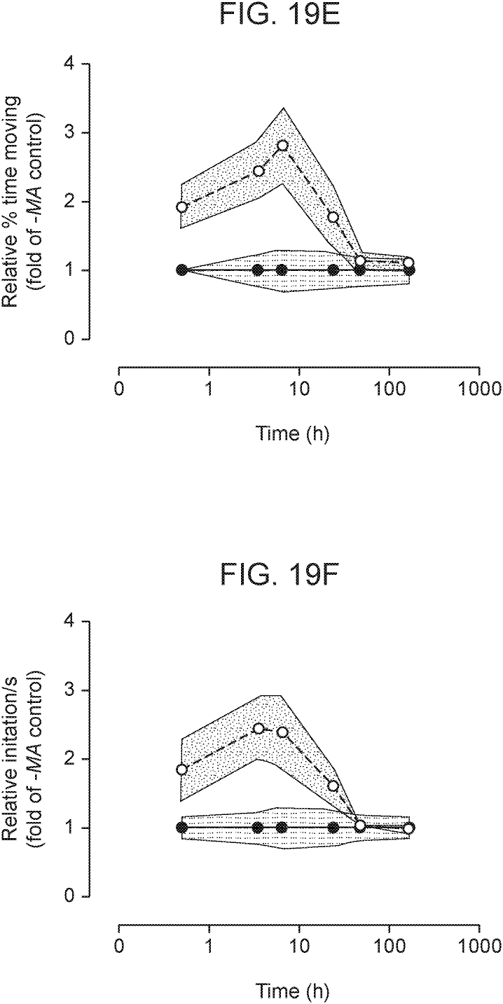

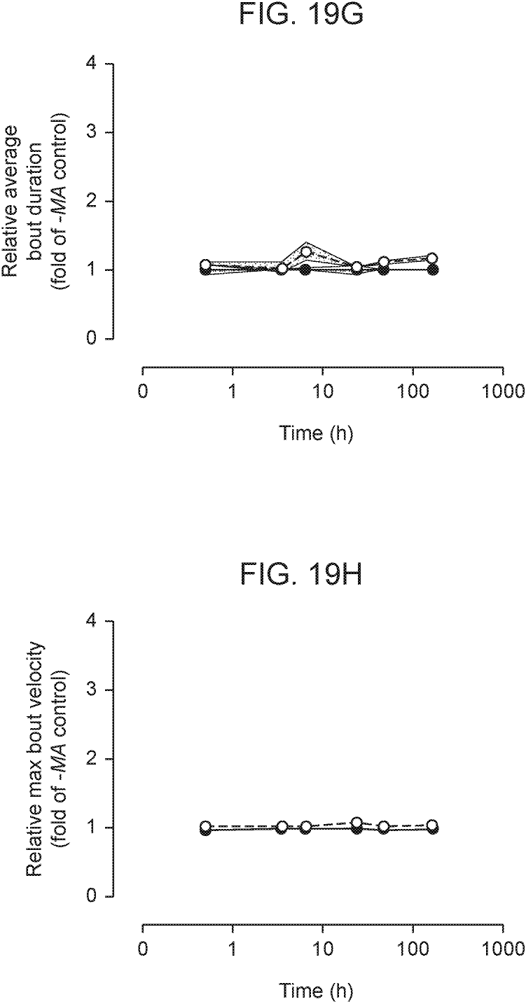

The longer-term effect of MP-D1_{ago} on dStr-dMSN D1Rs period was evaluated by infusing the active configuration of P-D1ago (trans), which is stable in the dark. The motor activity of mice expressing the M increased within 30 minutes of the start of infusion of trans-P-D1_{ago}, remained high for many hours, and returned to baseline over a two-day period (FIG. 19). The delayed activation may reflect the time it takes for P-D1ago to conjugate to the M. The slow decay of the trans-MP-D1_{ago} effect matches that observed with a membrane-anchored AMPA receptor DART antagonist in dStr-dMSNs, likely reflecting the turnover of the membrane anchor in these neurons. Thus, MP-D1_{ago} can be used to activate dStr-dMSN D1Rs either acutely or persistently.

FIGS. 3A-3G Activation of D1Rs in MSNs of the dStr with MP-D1_{ago} enhances movement. (A) D1R and its close homolog D5R are widely expressed in the brain (left panel) and are present in multiple cell types in the dorsal striatum (dStr; right panel). D1R is also expressed in the terminals of some glutamatergic afferents that innervate the striatum. dStr-dMSN D1Rs were selectively targeted with MP-D1_{ago} (red), which is composed of the membrane-anchor M_{EAAAK:ERE} (SEQ ID NO: 17) and P-D1_{ago}. (B) The M was virally delivered to dStr-dMSNs. Its expression (red = HA-tag staining) can be seen within the dStr. D-P1_{ago} and light were delivered the dStr with a reversible infusion/optical cannula. grey bar = 100 µm. blue = DAPI staining. (C) The M is present at the surface of dStr-dMSNs according to its ability to bind the impermeant SNAP-tag binding dye SNAP-Surface Alexa Fluor 647. Labeling was not observed in the absence of the M or dye. mVenus (green) is an indicator of viral infection, grey bars = 50 µM. (D) Locomotor activity of MP-D1_{ago} mice was measured in an open field. UV light and blue light were delivered to both sides of the brain. (E) The speed of mice with MP-D1_{ago} in dStr-dMSNs increased in response to a brief flash (450 nm, ~6 mW, 1 s) of blue light and returned to baseline after a brief flash (375 nm, ~9 mW, 1 s) of UV light. The behavioral response was not observed in mice lacking the M and/or P-D1ago (400 nL infusion in each hemisphere). (F) The speed of each mouse was averaged over the following two-minute periods: (i) just before exposure to blue light (UV pre), (ii) three minutes after exposure to blue light (blue), (iii) and one minute after exposure to the second flash of UV light (UV post). See FIG. 17A for more detail. Shown is a summary of the speed of -M or +M mice treated with vehicle or P-D1ago mice during the UV pre, blue, and UV post periods. RM one-way ANOVA, F-values from left to right: 0.2, 0.4, 0.1, 15.4, Bonferroni, *p < 0.05. n = 7 mice for each condition. (G) The behavioral photoeffect was not observed in the presence of the D1R antagonist SCH23390 (0.5 mg/kg).

FIGS. 4A-4G. dStr-dMSN D1R activation with MP-D1_{ago} enhances movement initiation but has no effect on movement duration. (A) Example movement dynamics of a mouse with MP-D1_{ago} in the inactive state (UV light; top panel) and active state (blue light; bottom panel), which correspond with the two-minute UV pre and blue periods described in FIG. 3f and FIG. 17A. The dotted lines represent the velocity threshold used to identify movement bouts. Movement initiations are indicated by black circles. (B) Summary of the movement dynamics of mice with MP-D1_{ago} under UV light and blue light. Shown for MP-D1_{ago} mice is the effect of UV light and blue light on the (C) average percent of the time in motion, (D) average initiations per second, (E) average movement bout duration, (F) the average maximum movement bout velocity, and (G) the percentage of the time that the mice were in the center of the open field. paired two-tailed t-test, p-values from left to right: **p < 0.01, **p < 0.01, 0.08, *p < 0.05, 0.21. n = 7 mice for each condition.

FIGS. 15A-15D. Expression of the membrane anchor component of MP-D1_{ago}, M_{EAAAK:ERE} (SEQ ID NO:17). (A) Schematic representation of AAV5-CAG-DIO-SP-HAM_{EAAAK:ERE} (SEQ ID NO: 17)-P2A-mVenus. The expression cassette is oriented in the forward direction in the presence of Cre-recombinase, which recognizes flox sites (black and white arrows). abbreviations: ITR = inverted terminal repeat, HA = hemagglutinin tag, SP = hemagglutinin signal peptide, WPRE = woodchuck hepatitis virus posttranscriptional regulatory element. (B) The AAV was injected into the dorsal striatum (dStr) of D1-Cre mice (sagittal on top and coronal on bottom). (C) Sagittal slices were stained with an anti-HA antibody. M_{EAAAK:ERE} expressed in direct pathway medium spiny neurons (dMSNs) of the dStr, which project to the substantia nigra reticulata (SNr). grey bar = 2 mm. blue = DAPI staining, red = HA-tag (M_{EAAAK}:_{ERE}; SEQ ID NO: 17) staining. (D) Coronal slices were stained with anti-D1R and anti-HA antibodies, which indicated that M_{EAAAK:ERE} (SEQ ID NO: 17) colocalizes with D1R in dMSNs. grey bar = 100 µm. red = HA-tag (M_{EAAAK}:_{ERE}; SEQ ID NO: 17) staining, blue = D1R staining.

FIGS. 16A-16G. Design and optimization of a bilateral infusion and dual color light delivery system for mouse brain. (A) A custom bilateral head implant that targets the dorsal striatum with interchangeable infusion and optical cannulas. The infusion cannula reaches the same depth as the AAV injection site (0.3 mm below the skull) and the optical cannula is placed above it (2.8 mm below the skull) to maximize the illumination of the AAV expression area. (B) Schematic of a laser system (Doric) that delivers two wavelengths of light from two fibers. The light from a UV laser (375 nm) and a blue laser (450 nm) are combined with a wavelength combiner, and both wavelengths are then divided into two fibers using a wavelength splitter. The splitter also serves as a rotary that reduces twining of the fibers, which are attached to the head of a mouse via the custom head implant described above. (C) MP-D1_{ago} requires two wavelengths of light: UV light to turn it off (left) and blue light to turn it on (right). If these wavelengths are not calibrated, D1R will be heterogeneously activated and deactivated with MP-D1_{ago} across the brain. The efficiency of light propagation through brain tissue depends on wavelength¹⁰¹ and brain area¹⁰². (D) To measure the propagation of UV light and blue light through the striatum, an optical fiber was placed above and progressively lowered downwards through excised striatal tissue taken immediately from sacked mice. A light meter with a pinhole the size of the end of the optical fiber (200 µM) was placed below the tissue and was used to measure the efficiency of transmission (E) and the percent of the initial power density at the tip of the fiber (F), similar to previous work¹⁰³. As expected, the higher wavelength of light (blue) propagated through brain tissue more efficiently than the lower wavelength (UV)¹⁰¹. (G) The light output of each wavelength at the fiber tip was adjusted (375 nm laser = 9.3 mW; 450 nm laser = 6 mW) to achieve nearly identical intensities across striatal tissue. This analysis indicates that D1R can be maximally activated or deactivated with MP-D1_{ago} across ~2 mm of striatal tissue. Because only a brief flash of light is required to persistently turn on or off the receptor, heating effects associated with typical opsins are less likely¹⁰⁴.

FIGS. 17A-17E. Extended data for the motor effect of MP-D1_{ago}-induced activation of dStr-dMSN D1Rs. (A) D1-Cre mice expressing mVenus (-M) or M_{EAAAK:ERE} (SEQ ID NO: 17) and mVenus (+M) in dStr-dMSNs received bilateral dorsal striatum (dStr)-infusions (1 µL) of either vehicle or the inactive form of P-D1_{ago} (cis; 100 µM) three hours prior to being placed in an open field. Mice were exposed to UV light (5 s) approximately 15 minutes before the open field test to ensure that MP-D1_{ago} (the combination of M_{EAAAK:ERE} (SEQ ID NO: 17) and P-D1_{ago}) was in the off-state. MP-D1_{ago} was switched to the on-state with blue light (1 s) at zero minutes and switched back to the off-state with UV light (1 s) at five minutes. For the statistical analyses, the speed of each mouse was averaged over the following two-minute periods: (i) just before exposure to blue light (UV pre), (ii) three minutes after exposure to blue light (blue), (iii) and one minute after exposure to the second flash of UV light (UV post). (B) Representative locomotor tracks of +M mice treated with vehicle or P-D1ago during the UV pre, blue, and UV post periods. Vehicle and P-D1ago tracks are shown for a single mouse. (C) Summary of the AAV expression profile and cannula locations in -M or +M mice. Each color is associated with a single mouse within a group. (D) The motor effect of MP-D1_{ago} in dStr-dMSNs is repeatable. n = 4 mice. (E) Summary of the average speed of MP-D1_{ago} mice treated with SCH23390 (0.5 mg/kg) during the UV pre, blue, and UV post periods. SCH23390 was administered 30 minutes before MP-D1_{ago} was switched to the on-state with blue light. RM one-way ANOVA, F-values from left to right: 1.0, 1.2, Bonferroni. There was no significant difference between any condition. n = 7 mice for each condition.

FIGS. 18A-18F. Extended data for the movement effects of dStr-dMSN D1R activation with MP-D1_{ago}. (A) Example movement dynamics of a vehicle treated M-expressing mouse in the inactive state (UV light; top panel) and active state (blue light; bottom panel), which correspond with the two-minute UV pre and blue periods described in FIG. 3F and FIG. 17A. The dotted lines represent the velocity threshold used to identify movement bouts. (B) Summary of the movement dynamics of vehicle treated M-expressing mice under UV light and blue light. Shown for vehicle treated M-expressing mice is the effect of UV light and blue light on (C) average percent of the time in motion, (D) average initiations per second, (E) average movement bout duration, and (F) the average maximum movement bout velocity, paired two-tailed t-test, p-values from left to right: 0.99, 0.94, 0.18, 0.55. n from left to right: 7, 7, 6, 6 mice. For FIGS. 18E and 18F, mice were excluded if there were no movement bouts under UV light.

FIGS. 19A-19D. MP-D1_{ago} can be used to chronically activate dStr-dMSN D1Rs. (A) D1-Cre mice expressing mVenus (-M) or M_{EAAAK:ERE} (SEQ ID NO: 17) and mVenus (+M) in dStr-dMSNs received bilateral dStr-infusions of vehicle (1 µL) and were placed in an open field for 15 minutes. The following day, they received bilateral dStr-infusions of the active form of P-D1_{ago} (trans; 100 µM; 1 µL) and were placed in the open field 0.5, 3.5, 6.5, 24, 48, and 168 hours later for 15 minutes each time. Shown is the time course of locomotion of the group (B) and individual animals (C) in response to vehicle or P-D1_{ago}. Distanced traveled was normalized to the mean distance traveled of the -M group at each time point, one-way ANOVA, F = 6.3, Bonferroni, *p < 0.05, **p < 0.01. n = 4 mice in the -M group and n = 4-5 mice in the +M group. (D) Summary of the AAV expression profile and cannula locations in -M or +M mice. Each color is associated with a single mouse within a group.

### Comparison of MP-D1_{ago} with optogenetic activation of a dopaminergic projection to the dorsal striatum

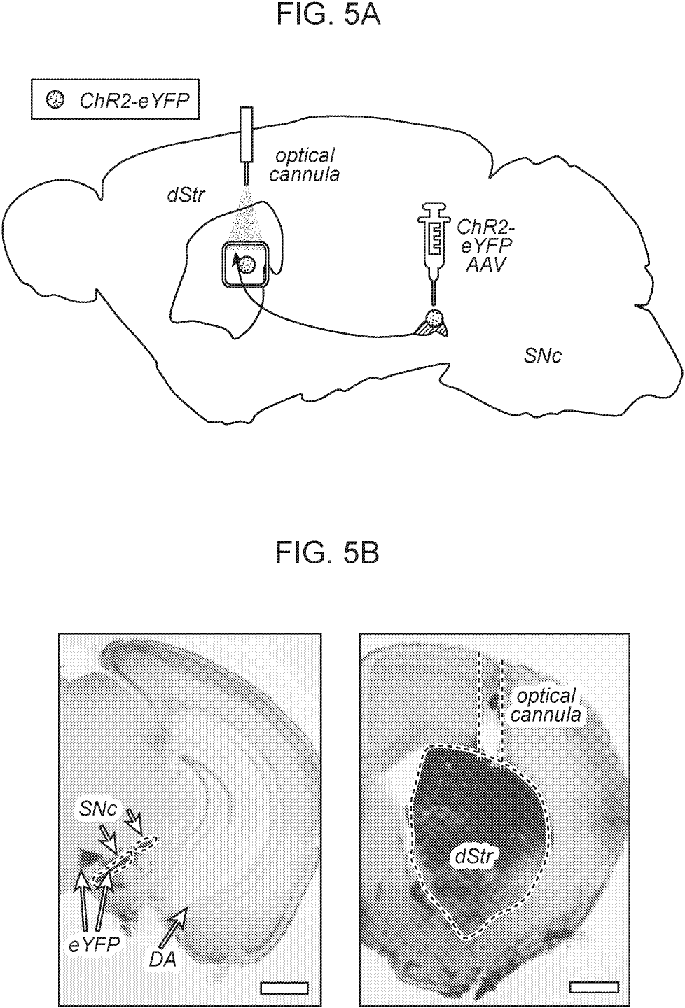

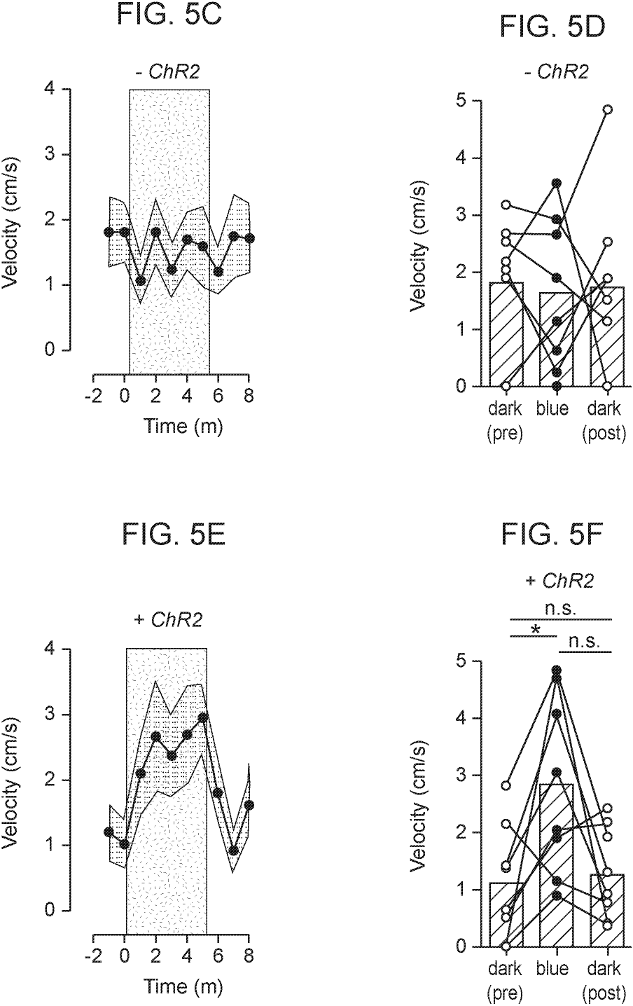

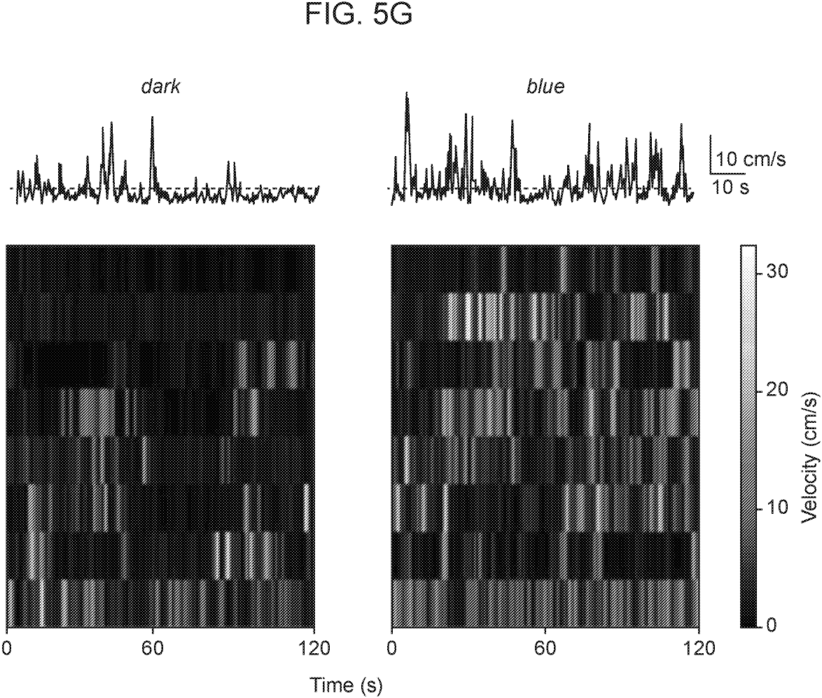

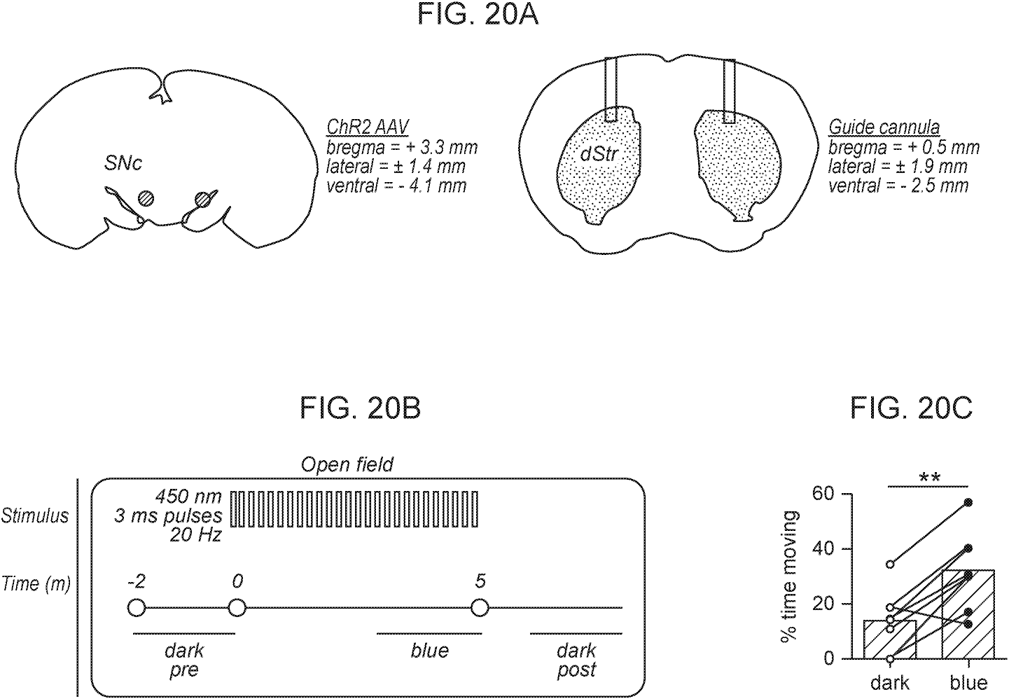

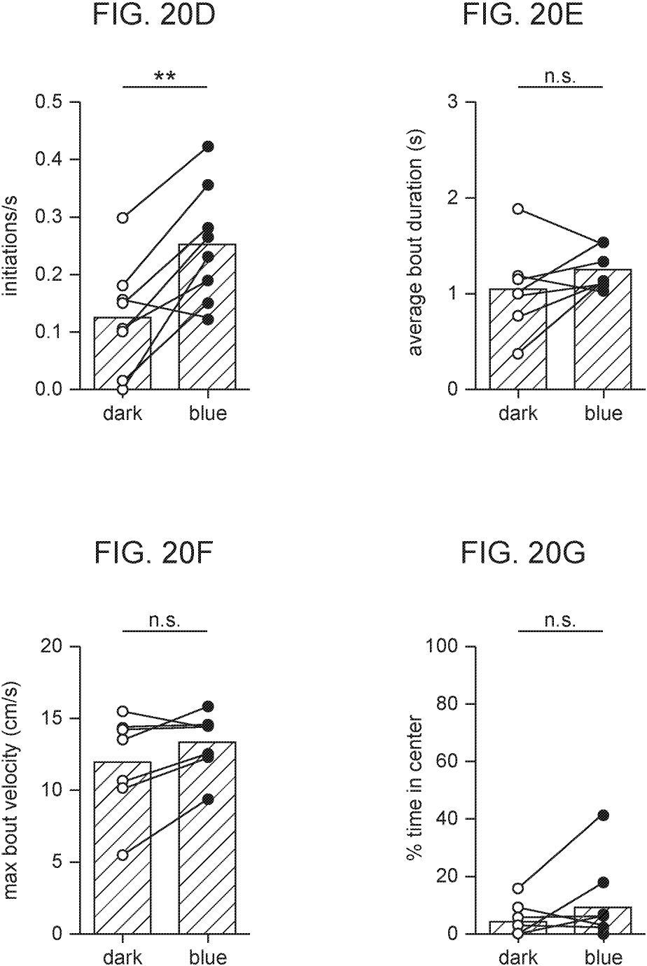

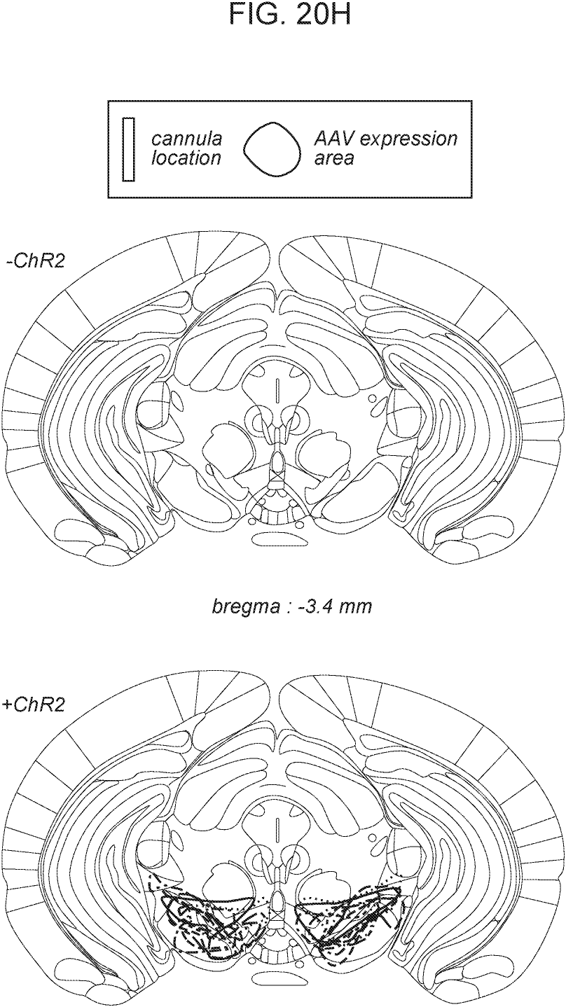

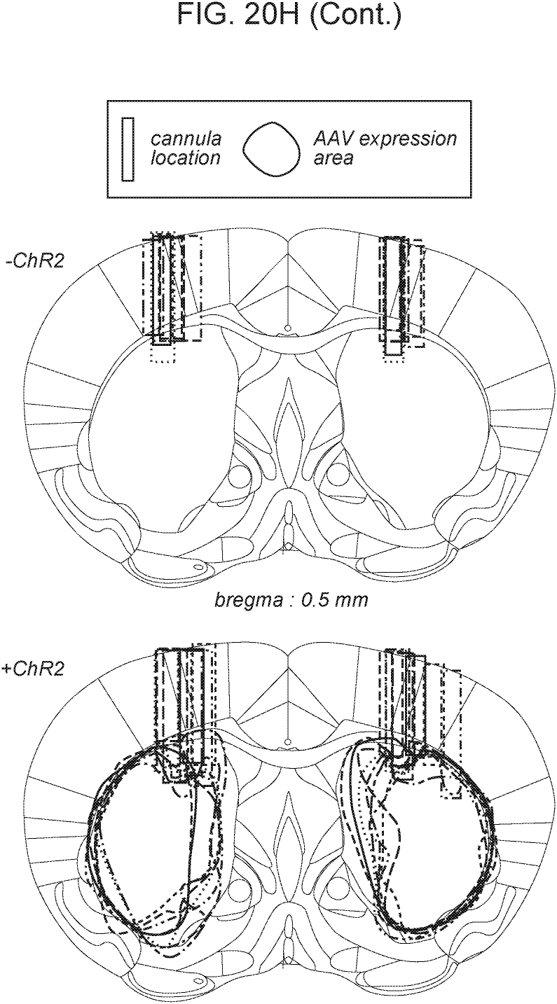

It was asked how the behavioral effect of activation of dStr-dMSN D1Rs with MP-D1_{ago} compares with that elicited by activity in dopaminergic inputs to the dStr, which is expected to activate not only dMSN D1Rs but also DARs in each cell type of the dStr, as well as GABA receptors. To test this, a Cre-dependent AAV encoding ChR2-eYFP was injected into the SNc of DAT-Cre mice. Robust eYFP expression was observed in SNc DA neurons, the major DA input to the dStr³⁶, and in the dStr, where SNc DA neurons make their axon terminal beds (FIG. 5B; and FIG. 20A). Activating ChR2 in SNc DA axon terminals in the dStr enhanced locomotion in the open field (FIGS. 5C-5F; and FIG. 20B). The enhanced locomotion was due to an increase in movement initiation, with no change in the duration of movement bouts (FIG. 5G; and FIGS. 20C-20F). The timing and magnitude of these effects closely resembled those elicited by photo-activation of dStr-dMSN D1Rs with MP-D1_{ago} (FIGS. 5H and 5I). This suggests that activation of dStr-dMSN D1Rs accounts for a significant portion of the ability of SNc DA neurons to enhance movement.

### Comparison of MP-D1_{ago} with a conventional D1R agonist

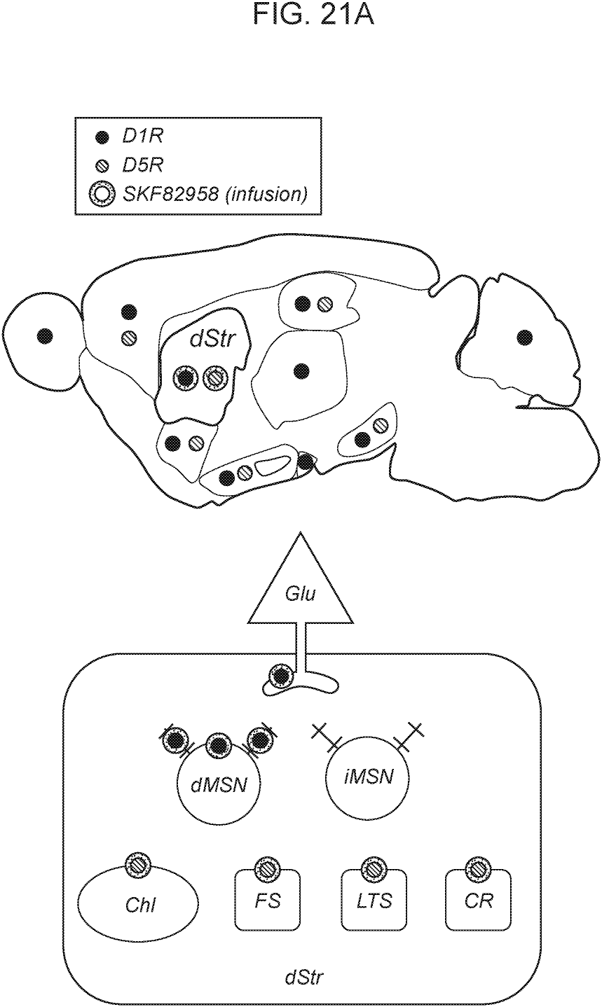

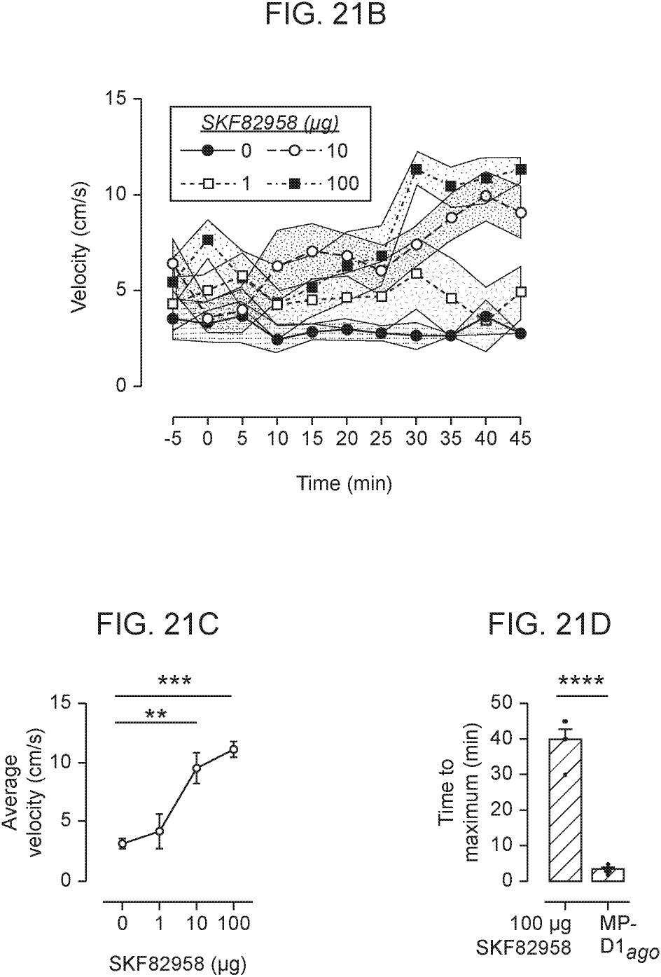

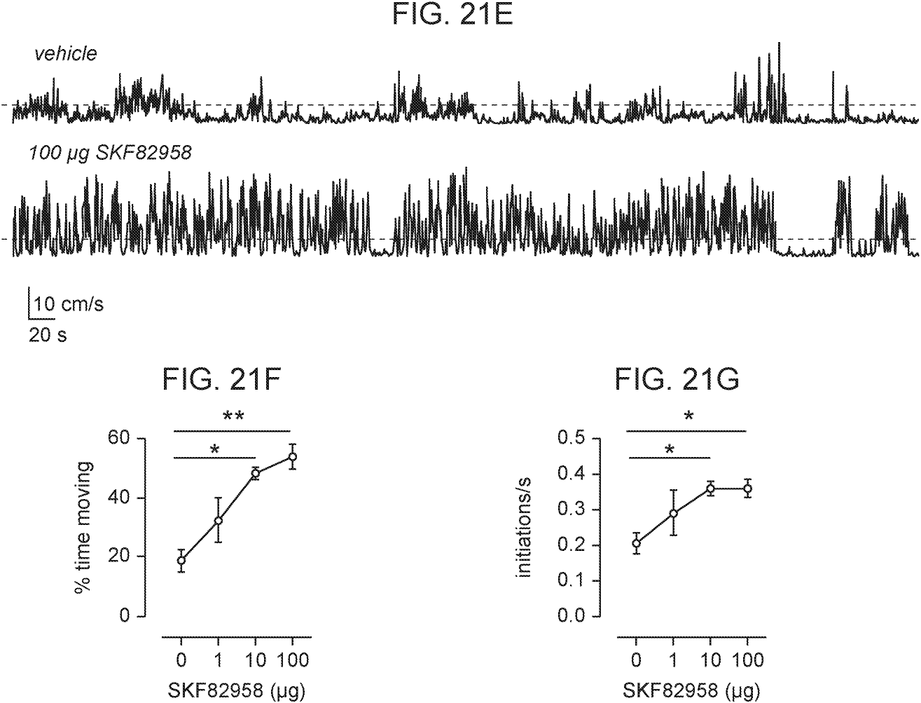

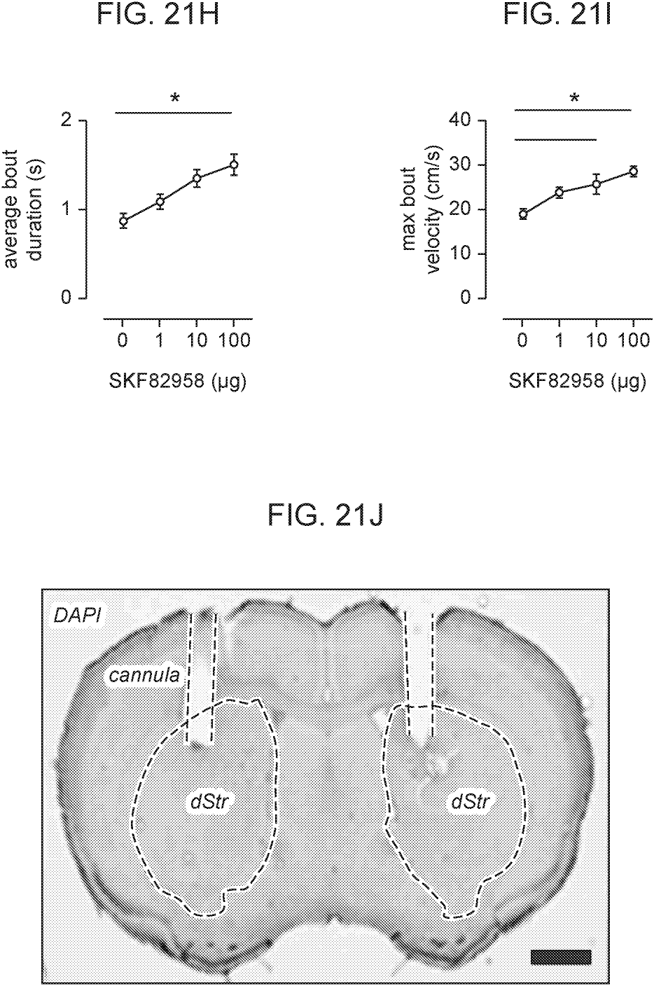

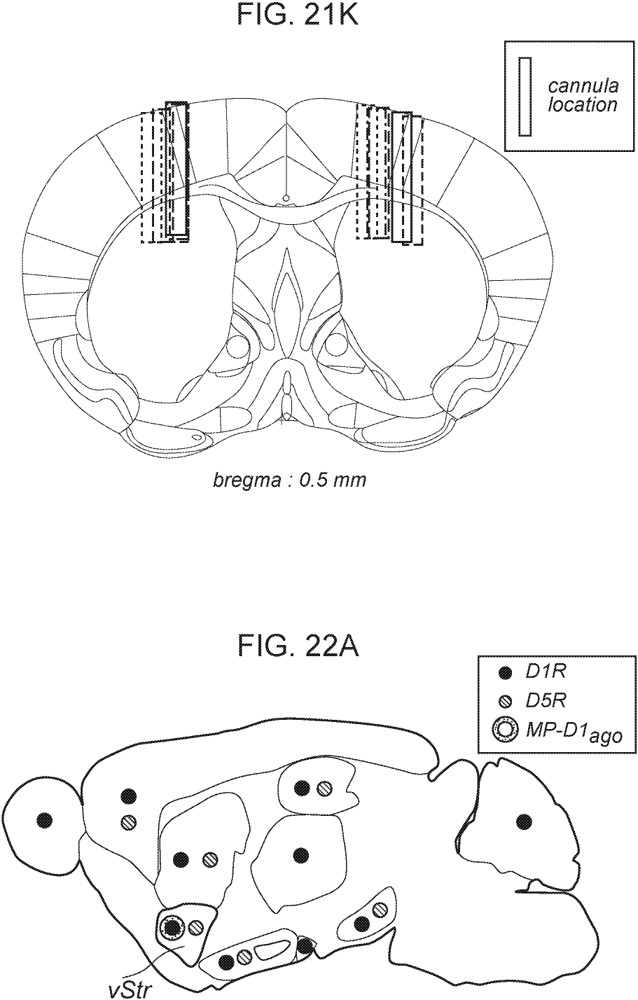

The selective activation of D1R in dStr-dMSNs was compared with MP-D1_{ago} to infusion into the dStr of the conventional full D1R agonist SKF82958, which is expected to also activate D5R in multiple cell types. Like MP-D1_{ago}, SKF82958 enhanced locomotion (FIGS. 21B-21C). However, locomotion increased ~12-times faster with MP-D1_{ago} than with SKF82958 (FIG. 21D), reflecting the temporal difference between light propagation (fast) and pharmacologic diffusion (slow) in brain tissue. Moreover, although both increased the percentage of the time that the mice were in motion (FIGS. 21E and 21F), MP-D1_{ago} selectively increased the probability of movement initiation (FIG. 4), whereas SKF82958 increased both movement initiation and the duration of the movement bouts (FIGS. 21G and 21H). This difference indicates that locally infused DAR ligands may not faithfully mimic the actions of dopaminergic input, either because of differences in the activation different DAR subtypes and cell types or because they diffuse to other brain areas (FIG. 21A).

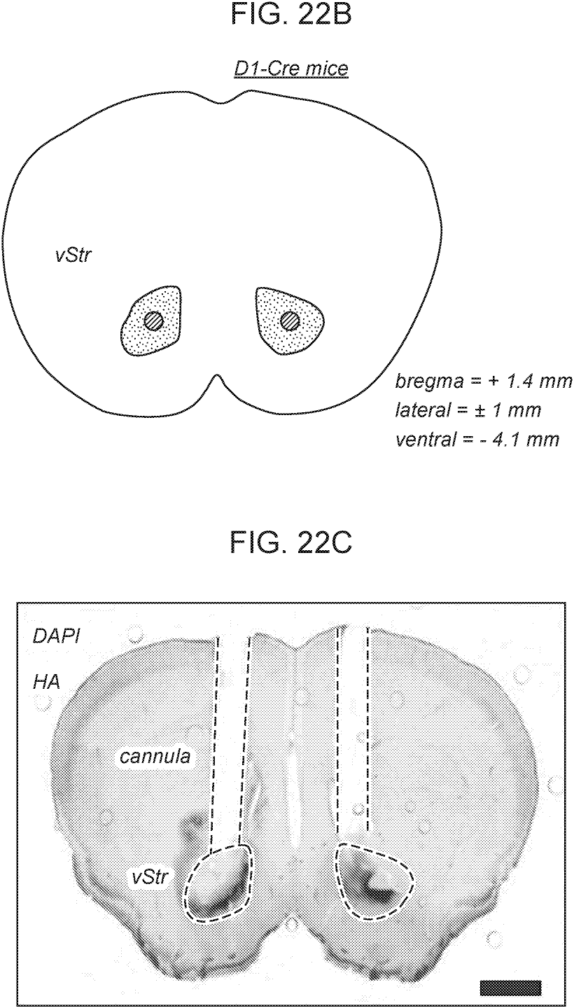

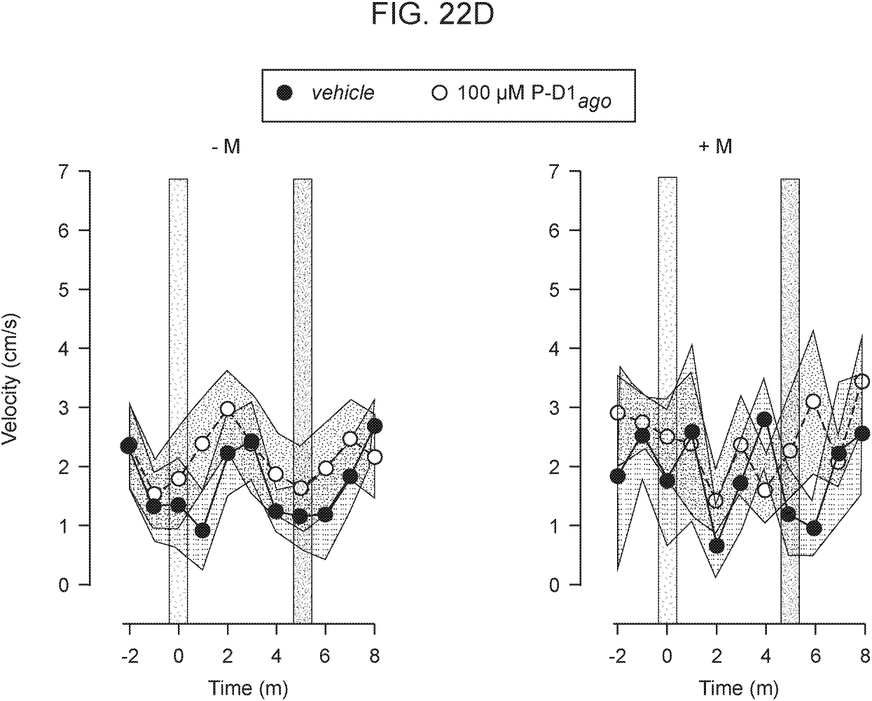

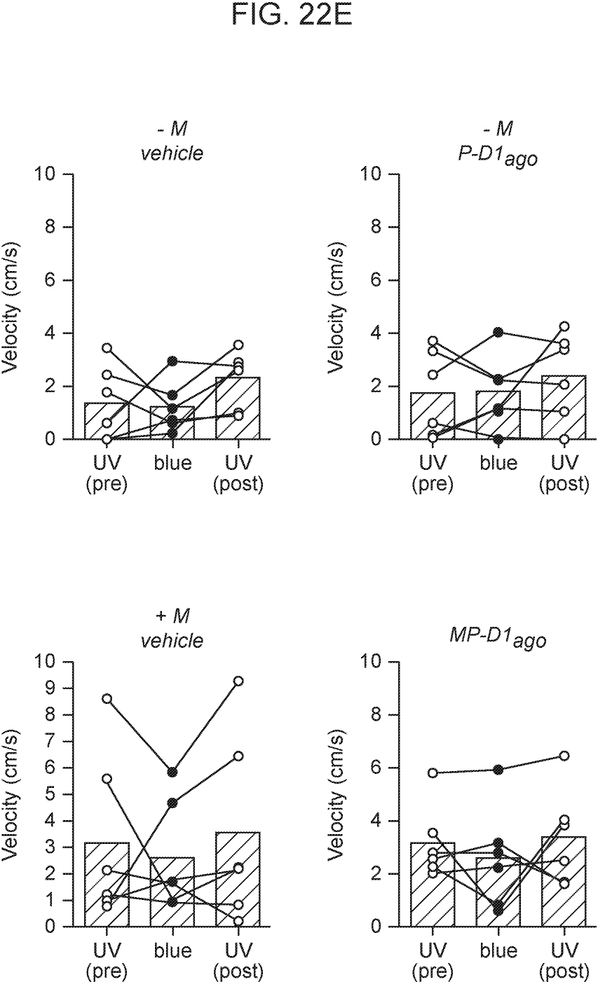

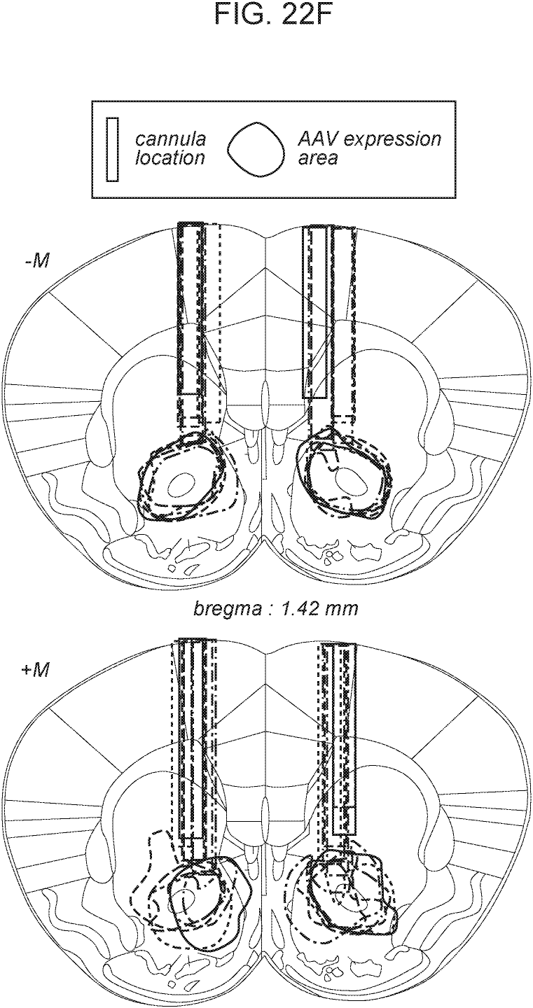

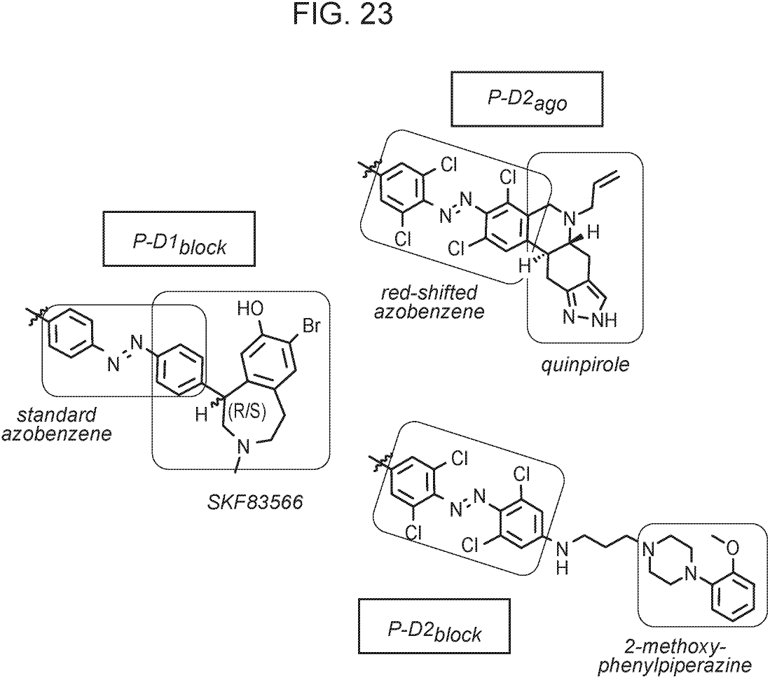

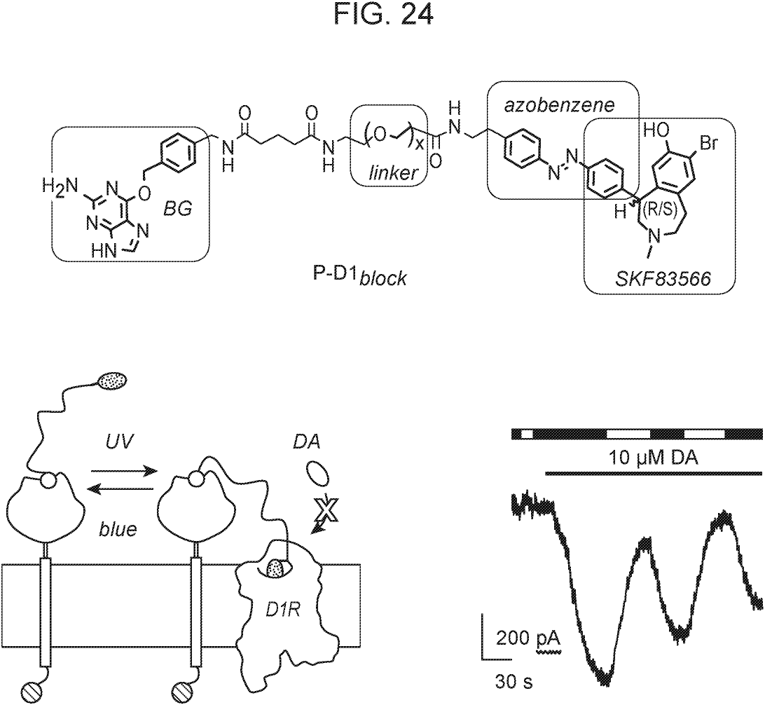

### D1Rs in the Nucleus Accumbens Have No Effect on Movement

Whereas the dStr is specialized for movement control, the nucleus accumbens (NAc) is associated with reward and motivation. However, studies suggest that dopaminergic input from the ventral tegmental area (VTA) to the NAc can also promote movement, possibly by increasing motivation and decreasing work-related response costs or promoting motor learning. D2R in indirect pathway-MSNs of the NAc (NAc-iMSNs) promotes locomotion in an open field⁴¹. By contrast, the effect of D1R in the NAc on locomotion is less clear because existing methods have produced contradictory results. To explore this, the effect of MP-D1_{ago} photo-activation in dMSNs of the NAc (NAc-dMSNs) was assessed. No effect on locomotion of switching fiber illumination from UV to blue light was observed (FIG. 22), suggesting that motor activation in the NAc is driven by iMSN D2R but not dMSN D1R.

FIGS. 5A-5I. Comparison of the motor effect of optogenetic activation of SNc terminals in the dStr with ChR2 to dStr-dMSN D1R activation with MP-D1_{ago}. (A) A Cre-dependent AAV encoding channelrhodopsin-2 (ChR2), AAV-EF1a-DIO-hChR2(H134R)-eYFP, was bilaterally injected into the substantia nigra compacta (SNc) of DAT-Cre mice, resulting in ChR2-eYFP expression in SNc DA neurons. To enhance neurotransmitter release from SNc DA neuron terminals onto the dorsal striatum (dStr), a bilateral optical cannula was implanted in the dStr. (B) Expression of ChR2-eYFP in DA neurons in SNc (left panel) and their terminals in the dStr (right panel). grey bars = 1 mm. blue = DAPI staining, green = eYFP (ChR2) staining. (C) The speed of mice lacking ChR2 was unaffected by blue light (450 nm, ~7 mW, 3 ms pulses at 20 Hz). (D) The speed of each mouse was averaged over the following two-minute periods: (i) just before exposure to blue light (dark pre), (ii) three minutes after exposure to blue light (blue), (iii) and one minute after exposure to blue light (dark post). See FIG. 20B for more detail. Shown is the summary of the speed of mice lacking ChR2 during the dark pre, blue, and dark post periods. RM one-way ANOVA, F = 0.1, Bonferroni. n = 8 mice. (E) The speed of mice with ChR2 in SNc DA neuron terminals increased in response blue light and returned to baseline in the dark. (F) Summary of the speed of ChR2 mice during the dark pre, blue, and dark post periods. RM one-way ANOVA, F = 6.3, Bonferroni, ^{∗}p < 0.05. n = 8 mice. (G) Example movement dynamics of a mouse with ChR2 in the inactive state (dark) and active state (blue light), which correspond with the dark pre and blue periods described above (top panels). The dotted lines represent the velocity threshold used to identify movement bouts. Summary of the movement dynamics of mice with ChR2 in the dark and in response to blue light (bottom panels). (H) The behavioral response to ChR2 activation in SNc DA neuron terminals in the dStr versus dStr-dMSN D1R activation with MP-D1_{ago}. ChR2 data from FIG. 5e and MP-D1_{ago} data from FIG. 3e were normalized to baseline movement and replotted. There was no significant difference between groups. RM two-way ANOVA. p = 0.99. (I) Summary of the relative difference in movement initiation, bout duration, and maximum bout velocity for mice with ChR2 or MP-D1_{ago} in their active and inactive states. ChR2 data from FIGS. 20D-20F and MP-D1_{ago} data from FIGS. 4d-f were normalized to baseline and replotted.

FIGS. 20A-20H. Extended data for the motor effect of optogenetic activation of SNc terminals in the dStr with ChR2. (A) Coronal sections indicating the ChR2 AAV injection site in the SNc (green circle) and the guide cannula implant site in the dStr (black rectangle). Control mice were implanted with a guide cannula but did not receive an AAV injection. (B) Shown is a schematic representation of the behavioral paradigm used to test movement in the ChR2 mice. The mice were placed in an open field and stimulated for five minutes with blue light (450 nm, ~7 mW at fiber tip, 3 ms pulses, 20 Hz). For the statistical analyses below, the speed of each mouse was averaged over the following two-minute periods: (i) just before exposure to blue light (dark pre), (ii) three minutes after exposure to blue light (blue), (iii) and one minute after exposure to blue light (dark post). Shown for ChR2 mice during the dark pre and blue periods is (C) average percent of the time in motion, (D) average initiations per second, (E) average movement bout duration, (F) the average maximum movement bout velocity, and (G) the percentage of the time that the mice were in the center of the open field. paired two-tailed t-test, p-values from left to right: ^{∗∗}p < 0.01, ^{∗∗}p < 0.01, 0.22, 0.07, 0.21 n from left to right: 8, 8, 7, 7, 8 mice. For FIGS. 20E and 20F mice were excluded if there were no movement bouts in the dark. (H) Summary of the AAV expression profile and/or cannula locations of mice with and without ChR2. Each color is associated with a single mouse within a group.

FIGS. 21A-21K. Comparison of the motor effect of a dStr-infusion of the full D1R agonist SKF82958 and dStr-dMSN D1R activation with MP-D1_{ago}. (A) An infusion of SKF82958 directly to the dorsal striatum (dStr) activates D1R and D5R in various cell types of this brain area and could diffuse into other brain areas. (B) Time course of the effect of a bilateral dStr-infusion of SKF82958 (400 nL per hemisphere) on the locomotion of D1-Cre mice in an open field. (C) Average velocity in the last 10 minutes of the open field test. one-way ANOVA, F = 14.3, Bonferroni, ^{∗∗}p < 0.01, ^{∗∗∗}p < 0.001. n = 5 mice per condition. (D) Time to maximum locomotion with SKF82958 or MP-D1_{ago}. unpaired two-sided t-test, ^{∗∗∗∗}p < 0.0001. n = 5 for SKF82958, n = 7 mice for MP-D1_{ago}. (E) Example movement dynamics of a mouse treated with vehicle or 100 µg SKF82958. Shown for SKF82958-treated mice is the (F) average percent of the time in motion, (G) average initiations per second, (H) average movement bout duration, and (I) the average maximum movement bout velocity. RM one-way ANOVA, F-values from left to right: 17.67, 8.61, 6.97, 8.88. Bonferroni, ^{∗}p < 0.05, ^{∗∗}p < 0.01. n = 5 mice. (J) Representative coronal brain slice from a mouse infused with SKF82958. grey bar = 1 mm. blue = DAPI staining. (K) Summary of cannula locations for individual mice. Each color is associated with a single mouse.

FIGS. 22A-22F. Activation of D1Rs in MSNs of the vStr with MP-D1_{ago} has no effect on movement. (A) D1Rs in vStr-dMSNs were selectively targeted with MP-D1_{ago}. (B) Schematic of mouse brain (coronal section) highlighting the location of the ventral striatum (vStr). An AAV encoding mVenus or the membrane-anchor M_{EAAAK:ERE} (SEQ ID NO:17) and mVenus was injected into the vStr of D1-Cre mice (red dot). (C) Expression of M_{EAAAK:ERE} (SEQ ID NO:17) in the vStr. grey bar = 1 mm. blue = DAPI staining, red = HA-tag (M_{EAAAK:ERE}; SEQ ID NO:17) staining. (D) The speed of mice with MP-D1_{ago} in vStr-dMSNs increased in response to a brief flash of blue light (450 nm, ~6 mW, 1 s) and returned to baseline after a brief flash of UV light (375 nm, ~9 mW, 1 s). There was no effect under any condition. (E) The speed of each mouse was averaged over the following two-minute periods: (i) just before exposure to blue light (UVpre), (ii) three minutes after exposure to blue light (blue), (iii) and one minute after exposure to the second flash of UV light (UVpost). There was no significant difference between any condition. RM one-way ANOVA, F-values from left to right: 1.9, 0.6, 0.4, 0.8, Bonferroni. n = 6 mice for each condition. (F) Summary of the AAV expression profile and cannula locations in -M or +M mice. Each color is associated with a single mouse within a group.

### Example 2

A D1 photo-antagonist (“P-D1_{block}”), a D2-specific photo-agonist (“P-D2_{ago}”), and a D2-specific photo-antagonist (“P-D2_{block}”) were designed. The D2 switches (P-D2_{ago} and P-D2_{block}) are red-shifted to enable combined use with the D1 switches (e.g., P-D1_{block}) (FIG. 23).

P-D1_{block} was tested in HEK293 cells co-expressing the membrane anchor (M), SNAP-TM, along with D1R and the GIRK channel. The data are depicted in FIG. 24. Activation of D1R by 10 µM dopamine, to induce maximal activation of D1R, evoked a large inward GIRK current. This current was blocked by illumination at near-UV to induce isomerization of the azobenzene in P-D1_{block} from trans (obstructed) to cis (exposed / binding competent). Blue light reversed the block and block is reproducible.

FIG. 24. P-D1_{block} on HEK293 cells. Cells co-expressed the membrane anchor, M, D1R and the GIRK channel. High external K⁺; Vh=-80 mV.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising:
a) a conjugate comprising:
i) an affinity agent that forms a covalent bond with a self-labeling protein tag;
ii) a linker;
iii) a photoisomerizable group; and
iv) a ligand that binds to a target ligand-binding polypeptide; and
b) a fusion polypeptide, or a recombinant expression vector comprising a nucleotide sequence encoding the fusion polypeptide, wherein the fusion polypeptide comprises:
i) a self-labeling protein tag;
ii) a peptide linker; and
iii) a membrane-anchoring polypeptide.

2. The system of claim 1, wherein the affinity agent comprises benzylguanine.

3. The system of claim 1, wherein the affinity agent comprises chloroalkane.

4. The system of claim 1, wherein the affinity agent comprises benzylcytosine.

5. The system of any one of claims 1-4, wherein the photoisomerizable group comprises a moiety selected from an azobenzene, a cyclic azobenzene, an azoheteroarene, a fulgide, a spiropyran, a triphenyl methane, a thioindigo, a diarylethene, and an overcrowded alkene.

6. The system of any one of claims 1-5, wherein the ligand is an agonist, an antagonist, an allosteric modulator, or a blocker.

7. The system of any one of claims 1-6, wherein the target ligand-binding polypeptide is selected from a transcription regulator, an ion channel, a cation channel, a ligand-gated ion channel, a voltage-gated ion channel, a quorum sensor, a pheromone receptor, a neurotransmitter receptor, or a G-protein-coupled receptor.

8. The system of any one of claims 1-7, wherein the target ligand-binding polypeptide is a D1 dopamine receptor, a D2 dopamine receptor, a glutamate receptor, a metabotropic glutamate receptor, an ionotropic glutamate receptor, an ionotropic nicotinic acetylcholine receptor, an ionotropic GABA-A receptor, a metabotropic GABA-B receptor, a metabotropic dopamine receptor, an ionotropic purinergic P2X receptor, a metabotropic purinergic P2Y receptor, a metabotropic serotonin receptor, an ionotropic serotonin receptor, an ionotropic glycine receptor, a cation channel, a potassium channel, a calcium channel, a sodium channel, a proton channel, an anion channel, or a chloride channel.

9. The system of any one of claims 1-8, wherein the photoisomerizable group comprises an azobenzene that isomerizes in response to visible light.

10. The system of any one of claims 1-9, wherein the target ligand-binding polypeptide is a D1 dopamine receptor.

11. The system of claim 10, wherein the ligand is dopamine or a dopamine derivative or analog that functions as a D1 dopamine receptor agonist.

12. The system of claim 10, wherein the ligand is a positive allosteric modulator of the D1 dopamine receptor.

13. The system of any one of claims 1-12, wherein the linker comprises poly(ethylene glycol).

14. The system of any one of claims 1-13, wherein the self-labeling protein tag comprises:
a) an amino acid sequence having at least 80% amino acid sequence identity to the SNAP polypeptide amino acid sequence set forth in SEQ ID NO:1;
b) an amino acid sequence having at least 80% amino acid sequence identity to the CLIP polypeptide amino acid sequence set forth in SEQ ID NO:2; or
c) an amino acid sequence having at least 80% amino acid sequence identity to the HALO polypeptide amino acid sequence set forth in SEQ ID NO:3.

15. The system of any one of claims 1-14, wherein the fusion polypeptide comprises an endoplasmic reticulum (ER) export signal peptide.

16. The system of any one of claims 1-15, wherein the peptide linker comprises the amino acid sequence EAAAK (SEQ ID NO:13).

17. The system of any one of claims 1-16, wherein the nucleotide sequence encoding the fusion polypeptide is operably linked to a cell type-specific promoter.

18. The system of claim 16, wherein the promoter is a dopamine-1 receptor promoter.

19. A system comprising:
a) a conjugate comprising:
i) an affinity agent that forms a covalent bond with a self-labeling protein tag;
ii) a linker;
iii) a photoisomerizable group; and
iv) a ligand that binds to a D1 dopamine receptor; and
b) a fusion polypeptide, or a recombinant expression vector comprising a nucleotide sequence encoding the fusion polypeptide, wherein the fusion polypeptide comprises:
i) a self-labeling protein tag; and
ii) an antibody specific for the D1 dopamine receptor.

20. The system of claim 19, wherein the affinity agent comprises benzylguanine.

21. The system of claim 19, wherein the affinity agent comprises chloroalkane.

22. The system of claim 19, wherein the affinity agent comprises benzylcytosine.

23. The system of any one of claims 19-22, wherein the photoisomerizable group comprises a moiety selected from an azobenzene, a cyclic azobenzene, an azoheteroarene, a fulgide, a spiropyran, a triphenyl methane, a thioindigo, a diarylethene, and an overcrowded alkene.

24. The system of any one of claims 19-23, where the antibody is a nanobody or a single-chain Fv.

25. A conjugate comprising:
a) an antibody specific for a D1 dopamine receptor;
b) a photoisomerizable group; and
c) a ligand that binds to the D1 dopamine receptor.

26. The conjugate of claim 25, where the antibody is a nanobody or a single-chain Fv.

27. The conjugate of claim 25 or claim 26, wherein the photoisomerizable group comprises an azobenzene.

28. A method of modulating the activity of a target ligand-binding polypeptide, the method comprising:
a) contacting a cell comprising the target ligand-binding polypeptide with a system of any one of claims 1-23 or a conjugate of any one of claims 25-27; and
b) exposing the cell to light of a wavelength that isomerizes the photoisomerizable agent present in the conjugate.

29. The method of claim 28, wherein the cell is in an individual.

30. The method of claim 28 or claim 29, wherein the light is provided by an implantable light source.

31. The method of any one of claims 28-30, wherein the cell is a direct pathway medium spiny neuron, and wherein the ligand is dopamine or a dopamine derivative.

32. A method of treating Parkinson’s disease in an individual, the method comprising administering the conjugate of any one of claims 25-27 into the dorsal striatum of the individual.

33. The method of claim 32, comprising exposing the dorsal striatum to light of a wavelength that isomerizes the photoisomerizable agent present in the conjugate.

34. The method of claim 32 or 33, wherein the light is provided by an implantable light source.

35. A method of treating Parkinson’s disease in an individual, the method comprising administering the system of any one of claims 1-24 into the dorsal striatum of the individual.

36. The method of claim 35, comprising exposing the dorsal striatum to light of a wavelength that isomerizes the photoisomerizable agent present in the conjugate.

37. The method of claim 35 or 36, wherein the light is provided by an implantable light source.
